(19) United States
(12) Patent Application Publication
(10) Pub. No.: US 2023/0365595 A1
(43) Pub. Date: Nov. 16, 2023

(54) INHIBITORS OF KRAS(G12D)

(71) Applicant: Ranok Therapeutics (Hangzhou) Co. Ltd., Hangzhou (CN)

(72) Inventors: Weiwen Ying, Lexington, MA (US); Kevin P. Foley, Waltham, MA (US); Chenghao Ying, Shanghai (CN); Zhiyong Wang, Hangzhou (CN); Wei Yin, Lianyungang (CN); Yaya Wang, Hangzhou (CN); Yan Dai, Hangzhou (CN); Thomas Prince, Danville, PA (US)

(73) Assignee: Ranok Therapeutics (Hangzhou) Co. Ltd., Hangzhou (CN)

(21) Appl. No.: 18/196,454

(22) Filed: May 12, 2023

(30) Foreign Application Priority Data
May 13, 2022 (WO) PCT/CN2022/092880
Jun. 29, 2022 (WO) PCT/CN2022/102534

(51) Int. Cl. C07D 519/00

(52) U.S. Cl. CPC C07D 519/00

(57) ABSTRACT

Provided are small molecule inhibitors of the KRAS(G12D) mutant oncoprotein having the structural formula:

R⁰⁰F₂C—L—R⁰

and pharmaceutically acceptable salts and compositions thereof, which are useful for treating cancers and related conditions.

### BACKGROUND

Discovered as a human oncogene in the early 1980s, the Kirsten rat sarcoma virus homolog (KRAS) gene encodes a monomeric small 21 kDa GTPase that has long been an elusive cancer drug target (Chang et al., PNAS, 1982, 79:4848-52; McCoy et al., Nature, 1983, 302:79-8). KRAS functions as a molecular switch for promoting cell growth by cycling between GTP- and GDP-bound states. In the GTP-bound state, KRAS signals for growth through the RAF-MAPK and PI3K-AKT-MTOR pathways. KRAS subsequently hydrolyzes GTP to GDP with the aid of GTPase activating proteins (GAPs). This GDP-bound state switches “off” KRAS pro-growth signaling. KRAS can then be switched back “on” by GDP to GTP exchange through the aid of guanine nucleotide exchange factors, such as SOS1 (Cox and Der, Small GTPases, 2010, 1:2-27; Kerk et al., Nat Rev Cancer, 2021, 21:510-525). Preventing this exchange by locking KRAS in the GDP-bound state is a practical method for inhibiting its growth promoting activity.

The human KRAS gene is encoded on Chromosome 12p12.1 and is among the most frequently mutated genes in human cancers (Pylayeva-Gupta et al., Nat Rev Cancer, 2011, 11:761-774). Mutations that prevent GTP-hydrolysis lock KRAS in the active GTP-bound state and reprogram cells for perpetual proliferation. KRAS mutated from glycine (G) at the 12th codon to aspartate (D) creates a chronically active KRAS(G12D) oncoprotein, the gene for which is observed in 6.8% of cancers cases as analyzed by next-generation sequencing (Zhou et al., Pathol Oncol Res, 2020, 26:2835-2837). In tumor type-specific studies, KRAS(G12D) is associated with poor clinical outcomes and observed in 17% of lung, 14.3% of colorectal, and 48% of pancreatic tumors (Aredo et al., Lung Cancer, 2019, 133:144-150; Olmedillas-Lopez et al., World J Gastroenterol, 2017, 23(39):7087-709; Miglio et al., Pathol Res Pract, 2014, 210:307-11; Gou et al., Br J Cancer, 2020, 22:857-867), among other cancers. Historically, oncogenic KRAS mutants have been considered undruggable (McCormick F, Biochem J, 2019, 476:356-74), however the discovery of an allosteric pocket in GDP-bound KRAS has allowed the search for small molecule inhibitors (Ostrem et al., Nature, 2013, 503: 548-51). The G12D mutation moreover provides a unique chemical moiety-binding space due to the encoding of an acidic amino acid residue (D) in place of a small flexible amino acid residue possessing only a hydrogen side chain (G). This alteration of the KRAS protein structure provides a unique space that may be targeted with small molecules drugs that specifically inhibit KRAS(G12D) oncogenic activity. It is therefore desirable to design and develop small molecule drugs that target KRAS(G12D) with sufficient bioavailability to treat diseases such as cancer.

### SUMMARY

Provided herein are small molecule inhibitors of the KRAS(G12D) mutant oncoprotein. Inhibitors of KRAS(G12D) include those having the structural formula I*:

R⁰⁰F₂C—L—R⁰ (I*)

and pharmaceutically acceptable salts and compositions comprising such, wherein L, R⁰, and It' are as defined herein. The use of these compounds, salts, and compositions for treating diseases responsive to the inhibition of KRAS(G12D), such as cancer, is also disclosed.

In one aspect, by virtue of the trifluoroacetyl group on the nitrogen atom of the diazabicylooctanyl, the disclosed compounds show improved bioavailability over non-trifluoroacetylated counterparts and/or derivatives. See e.g., Table 2 which shows that introduction of the trifluoroacetyl group on the nitrogen atom of the diazabicylooctanyl resulted in at least at 15-fold increase in %F for inventive compounds.

### BRIEF DESCRIPTION OF THE DRAWINGS

FIG. 1. shows the results from an in vivo xenograft tumor model using 75 mg/kg Compound 3 dosed orally for over 3 weeks.

### DETAILED DESCRIPTION

1. General Description of Compounds

As part of a first embodiment, provided is a compound of the structural formula I*:

R⁰⁰F₂C—L—R⁰ (I*);

or a pharmaceutically acceptable salt thereof, wherein L is selected from —C(═O), —S(═O), and —S(O)₂; R⁰ is a chemical entity which binds to KRAS^{G12D}; and R⁰⁰ is selected from hydrogen, F, Cl, and CF₃.

2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein the term “comprising” or “comprises” are used in reference to compositions, methods, and respective component(s) thereof, that are present in a given embodiment, yet open to the inclusion of unspecified elements.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-l methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkenyl” means a saturated straight chain or branched non-cyclic hydrocarbon having, unless specified otherwise, from 2 to 10 carbon atoms (e.g., (C₂-C₆)alkenyl or (C₂-C₄)alkenyl) and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-l-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-l-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

The term “aryl” used alone or as part of a larger moiety as in “—(C₁-C₄)alkylaryl”, refers to monocyclic and bicyclic carbon ring systems having a total of six to 10 ring members, wherein at least one ring in the system is aromatic. Examples include, but are not limited to phenyl, naphthyl, anthracyl and the like. It will be understood that when specified, optional substituents on an aryl group may be present on any substitutable position.

As used herein, the term “cycloalkyl” means a saturated, monocyclic alkyl radical having from e.g., 3 to 10 carbon atoms (e.g., from 3 to 6 carbon atoms). Representative cycloalkyls include cyclopropyl, cyclobutyl, cyclopentyl, cyclohexyl, cycloheptyl, cyclooctyl, cyclononyl, and cyclodecanyl.

The term “oxo” refers to the group ═O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

As used herein, the term “halogen” or “halo” means F, Cl, Br or I.

As used herein, the term “heterocyclyl” means a 4- to 12-membered monocyclic or polycyclic saturated or partially unsaturated heterocyclic ring containing 1 to 4 heteroatoms independently selected from N, 0, and S. The heterocycle may be attached via any heteroatom or carbon atom, as valency permits. Representative heterocycles include morpholinyl, thiomorpholinyl, pyrrolidinonyl, pyrrolidinyl, piperidinyl, piperazinyl, oxiranyl, dioxanyl, oxetanyl, dihydrofuranyl, dihydropyranyl, isoindolinyl, dihydropyridinyl, tetrahydrofuranyl, tetrahydropyranyl, tetrahydropyrindinyl, tetrahydropyrimidinyl, diazabicyclooctanyl, hexahydropyrrolizinyl, 2-azaspiro[3.3]heptanyl, 2,7-diazaspiro[3.5]nonanyl, 2-azaspiro[3.5]nonanyl, 3-azabicyclo[3.1.0]hexanyl, 8-azabicyclo[3.2.1]octanyl, 3,8-diazabicyclo[3.2.1]octanyl, 3,6-diazabicyclo[3.1.1]heptanyl, octahydro-1H-pyrrolo[2,3-c]pyridinyl, and the like. Optional substituents on a heterocyclyl group may be present on any substitutable position and, include, e.g., the position at which the heterocyclyl is attached, valence permitting.

As used herein, the term “heteroaryl” means a 5- to 12-membered aromatic radical containing 1-4 heteroatoms selected from N, O, and S. A heteroaryl group may be mono- or bicyclic. The heteroaryl may be attached via any heteroatom or carbon atom, as valency permits. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. Optional substituents on a heteroaryl group may be present on any substitutable position and, include, e.g., the position at which the heteroaryl is attached, valence permitting. When used in connection to describe a chemical group that may have multiple points of attachment, a hyphen (—) designates the point of attachment of that group to the variable to which it is defined. For example, —(C₁-C₄)alkylaryl and means that the point of attachment for these groups occurs on the alkyl group.

A hash bond as in “ [symbol] ” represents the point at which the depicted group is attached to the defined variable.

The term “KRAS” refers to the protein product of the KRAS proto-oncogene, GTPase gene.

The term “KRAS(G12D)” refers to the protein product of the KRAS gene carrying a mutation that results in the glycine amino acid at position 12 of KRAS being replaced by an aspartate.

A “chemical entity which binds KRAS^{G12D}” refers to a small molecule or a distinct portion of a larger molecule which binds to a portion of KRAS^{G12D}. In some aspects, the chemical entity which binds KRAS^{G12D} is a small molecule. In some aspects, the chemical entity which binds KRAS^{G12D} is a small molecule having a molecular weight of less than 2,000 g/mol. In some aspects, the chemical entity which binds KRAS^{G12D} induces a confirmation change in KRAS^{G12D}.

The term “SOS1” refers to the protein product of the SOS1 gene that functions as a guanine nucleotide exchange factor for RAS proteins.

The compounds described herein may have chiral centers and/or geometric centers (E- and Z-isomers). It will be understood that the present disclosure encompasses all stereoisomers and geometric isomers. Tautomeric forms of the compounds described herein are also part of the present disclosure.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the depicted stereoisomer plus the weight of the other stereoisomers.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts).

Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total). “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments, an agent is administered intravenously, intramuscularly, subcutaneously, intradermally, intranasally, orally, transcutaneously, or mucosally. In certain embodiments, an agent is administered intravenously. In In certain embodiments, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

As part of a second embodiment, the compound of structural formula I* is of the structural formula I:

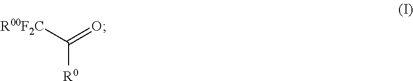

or a pharmaceutically acceptable salt thereof, wherein the variables are as described above for formula I*.

As part of a third embodiment, the compound of structural formula I* or I is of the structural formula Ia or Ib:

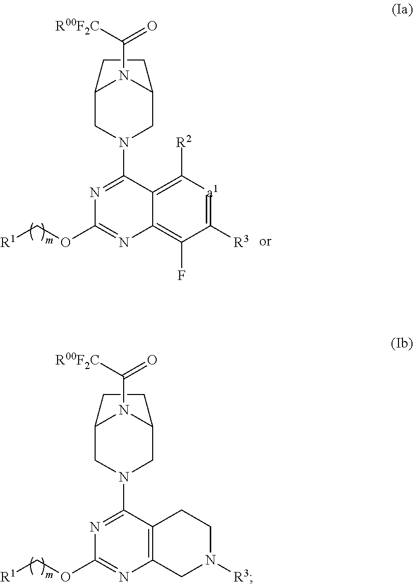

or a pharmaceutically acceptable salt thereof, wherein
R¹ is selected from (C₁-C₄)alkyl0(C₁-C₄)alkyl, -(C₁-C₄)alkylNH(C₁-C₄)alkyl, -(C₁-C₄)alkylN[(C₁-C₄)alkyl]₂, heterocyclyl, and cycloalkyl, wherein said heterocyclyl and cycloalkyl are each optionally and independently substituted;
R² is selected from hydrogen, (C₁-C₄)alkyl, (C₁-C₄)haloalkyl, (C₁-C₄)alkoxy, (C₁-C₄)haloalkoxy, (C₁-C₄)alkynyl, (C₁-C₄)alkenyl, halo, (C3-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl;
R³ is selected from

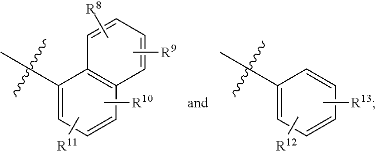

a¹ is N or CHZ¹;
Z¹ is selected from hydrogen, halo, (C₁-C₄)alkyl, (C₂-C₄)alkenyl, cyano, cyano(C₁-C₄)alkyl, —S[halo(C₁-C₄)alkyl], and (C₃-C₆)cycloalkyl (such as e.g., hydrogen, methyl, ethyl, isopropyl, cyano, —SCF₃, cyanoethyl, cyclopropyl, and 2-methylpropenyl);
R⁸, R⁹, R¹⁰, R¹¹, R¹², and R¹³ are each independently selected from hydrogen, (C₁-C₄)alkyl, (C₁-C₄)haloalkyl, (C₁-C₄)cyanoalkyl, (C₁-C₄)hydroxyalkyl, -(C₁-C₄)alkylNR^{a}R^{b}, -(C₁-C₄)alkylC(O)NR^{a}R^{b}, (C₁-C₄)alkyl0(C₁-C₄)alkyl, -(C₁-C₄)alkylC(O)OR^{a}, -(C₁-C₄)alkylNIVC(O)OR^{b}, —(C₁-C₄)alkylC(O)R^{a}, —(C₁-C₄)alkylheterocyclyl, -(C₁-C₄)alkylaryl, -(C₁-C₄)alkylheteroaryl, (C₂-C₄)alkenyl, (C₂-C₄)haloalkenyl, (C₂-C₄)cyanoalkenyl, (C₂-C₄)hydroxyalkenyl, —(C₂-C₄)alkenylNR^{a}R^{b}, (C₂-C₄)alkynyl, (C₂-C₄)haloalkynyl, (C₂-C₄)cyanoalkynyl, (C₂-C₄)hydroxyalkynyl, -(C₂-C₄)alkynylNR^{a}R^{b}, (C₁-C₄)alkoxy, (C₁-C₄)haloalkoxy, halo, cyano, oxo, hydroxy, —S(C₁-C₄)alkyl, —S(C₁—C₄)haloalkyl, —NR^{a}(O)R^{b}, —C(O)1V, —C(O)R^{a}, −SO₂R^{a}, —S(O)R^{a}, —SO₂NR^{a}R^{b}, —NRaSO₂R^{b}, (C₃-C₆)cycloalkyl, 5- or 6-membered heteroaryl, and 4- to 6-membered heterocyclyl, wherein said heterocyclyl, aryl, and heteroaryl of -(C₁-C₄)alkylheterocyclyl, -(C₁-C₄)alkylaryl, and -(C₁-C₄)alkylheteroaryl, and said (C₃-C₆)cycloalkyl, 5- or 6-membered heteroaryl, and 4- to 6-membered heterocyclyl are each optionally and independently substituted with 1 to 3 groups selected from R^{c},
m is 0, 1, or 2;
R^{a} and R^{b} are each independently selected from hydrogen, (C₁-C₄)alkyl, and (C₁-C₄)haloalkyl, or R^{a} and R^{b}, when on the same nitrogen atom, may be taken together to form a heterocyclyl; and
RC is selected from halo, (C₁-C₄)alkyl, (C₁-C₄)haloalkyl, (C₁-C₄)alkoxy, (C₁-C₄)haloalkoxy, cyano, hydroxyl, oxo, -C(O)OR^{a}, -C(O)R^{a}, —SO₂R^{a}, —S(O)R^{a}, —SO₂NR^{a}R^{b}, —NRac(O)R^{b}, —NR^{a}SO₂R^{b}, −NR^{a}R^{b}, and NO₂, wherein the remaining variables are as described above for formula I* or formula I.

As part of a fourth embodiment, le in the compound of the structural formula Ia or Ib, or a pharmaceutically acceptable salt thereof, is selected from an optionally substituted heterocyclyl and an optionally substituted cycloalkyl, wherein the remaining variables are as described above for structural formula I*, I, Ia or Ib. Alternatively, as part of a fourth embodiment, le in the compound of the structural formula Ia or Ib, or a pharmaceutically acceptable salt thereof, is selected from an optionally substituted fused bicyclic heterocyclyl and an optionally substituted (C₃-C₆)cycloalkyl, wherein the remaining variables are as described above for structural formula I*, I, Ia or Ib. In another alternative, as part of a fourth embodiment, le in the compound of the structural formula Ia or Ib, or a

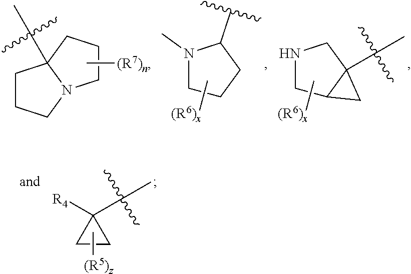

pharmaceutically acceptable salt thereof, is selected from
R⁴, R⁶, and R⁷ are each independently selected from hydrogen, (C₁-C₄)alkyl, (C₁-C₄)haloalkyl, (C₁-C₄)cyanoalkyl, (C₁-C₄)hydroxyalkyl, —(C₁-C₄)alkylNR^{a}R^{b}, —(C₁-C₄)alkylC(O)NR^{a}R^{b}, (C₁-C₄)alkylO(C₁-C₄)alkyl, -(C₁-C₄)alkylC(O)OR^{a}, —(C₁-C₄)alkylNR^{a}C(O)OR^{b}, —(C₁-C₄)alkylC(O)R^{a}, -(C₁-C₄)alkylheterocyclyl, —(C₁-C₄)alkylaryl, -(C₁-C₄)alkylheteroaryl, (C₂-C₄)alkenyl, (C₂-C₄)haloalkenyl, (C₂-C₄)cyanoalkenyl, (C₂-C₄)hydroxyalkenyl, —(C₂-C₄)alkenylNR^{a}R^{b}, (C₂-C₄)alkynyl, (C₂-C₄)haloalkynyl, (C₂-C₄)cyanoalkynyl, (C₂-C₄)hydroxyalkynyl, —(C₂-C₄)alkynylNR^{a}R^{b}, (C₁-C₄)alkoxy, (C₁-C₄)haloalkoxy, halo, cyano, oxo, hydroxy, —S(C₁-C₄)alkyl, —S(C₁-C₄)haloalkyl, —NR^{a}R^{b}, —NR^{a}C(O)R^{b}, —C(O)R^{a}, —C(O)OR^{a}, —SO₂R^{a}, —S(O)R^{a}, —SO₂NR^{a}R^{b}, _NR^{a}SO₂R^{b}, (C3_{—}

C₆)cycloalkyl, 5- or 6-membered heteroaryl, and 4- to 6-membered heterocyclyl, wherein said heterocyclyl, aryl, and heteroaryl of -(C₁-C₄)alkylheterocyclyl, -(C₁-C₄)alkylaryl, and -(C₁-C₄)alkylheteroaryl, and said (C₃-C₆)cycloalkyl, 5- or 6-membered heteroaryl, and 4- to 6-membered heterocyclyl are each optionally and independently substituted with 1 to 3 groups selected from R^{c};
R⁵ is (C₁-C₄)alkyl or halo; and
n, x and z are each independently 0, 1, or 2, wherein the remaining variables are as described above for structural formula I*, I, Ia or Ib. In another alternative, as part of a fourth embodiment, le in the compound of the structural formula Ia or Ib, or a pharmaceutically acceptable salt thereof, is selected from

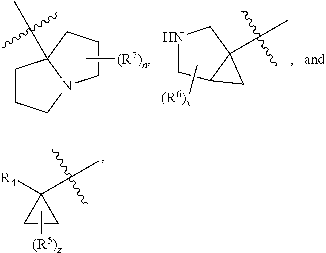

wherein the remaining variables are as described above and for structural formula I*, I, Ia or Ib.

As part of a fifth embodiment, the compound of structural formula I*, I, Ia or Ib is of the structural formula Ia':

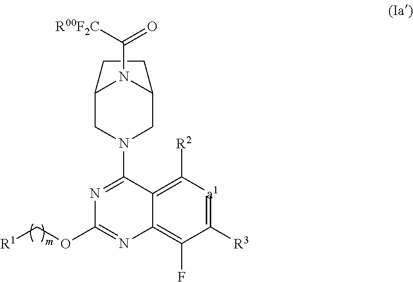

or a pharmaceutically acceptable salt thereof, wherein the remaining variables are as described above for structural formula I*, I, Ia, Ib or the fourth embodiment.

As part of a sixth embodiment, al in the compound of structural formula Ia or Ia', or a pharmaceutically acceptable salt thereof, is N, wherein the remaining variables are as described above for structural formula Ia or Ia′ or the fourth embodiment.

As part of a seventh embodiment, R° ′ in the compound of structural formula I*, I, Ia, Ia′, or Ib, or a pharmaceutically acceptable salt thereof, is F, wherein the remaining variables are as described above for structural formula 1*, I, Ia, Ia′, or Ib or the fourth or sixth embodiment.

As part of an eighth embodiment, R³ in the compound of structural formula Ia, Ia′, or Ib, or a pharmaceutically acceptable salt thereof, is

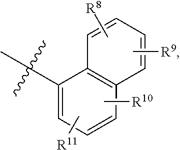

wherein the remaining variables are as described above for structural formula Ia, Ia′, or Ib or any one of the fourth, sixth, or seventh embodiments.

As part of a ninth embodiment, R⁸, R⁹, R¹⁰, and R¹¹ in the compounds of structural formula Ia, Ia′, or Ib, or a pharmaceutically acceptable salt thereof, are each independently selected from hydrogen, halo, hydroxyl, and (C₂-C₄)alkynyl, wherein the remaining variables are as described above for formula Ia, Ia′, or Ib or any one of the fourth or sixth to eighth embodiments. Alternatively, as part of a ninth embodiment, R⁸, R⁹, R¹⁰, and R¹¹ in the compounds of structural formula Ia, Ia′, or Ib, or a pharmaceutically acceptable salt thereof, are each hydrogen; or R⁹, R¹⁰ and R¹¹ are each hydrogen and R⁸ is selected from halo, hydroxyl, and (C₂-C₄)alkynyl; or R¹⁰ R¹¹ are each hydrogen and R⁸ and R9 are each independently selected from halo, hydroxyl, and (C₂-C₄)alkynyl; or R¹¹ is hydrogen and R⁸, R⁹, and R¹⁰ are each independently selected from halo, hydroxyl, and (C₂-C₄)alkynyl, wherein the remaining variables are as described above for formula Ia, Ia′, or Ib or any one of the fourth or sixth to eighth embodiments. In another alternative, as part of an ninth embodiment, R⁸, R⁹, R¹⁰ and R¹¹ in the compounds of structural formula Ia, Ia′, or Ib, or a pharmaceutically acceptable salt thereof, are each hydrogen; or R⁹, R¹⁰ and R⁹¹¹ are each hydrogen and R⁸ is (C₂-C₄)alkynyl; or R¹⁰ and R¹¹ are each hydrogen, R⁸ is (C₂-C₄)alkynyl, and R⁹ is halo; or R¹¹ is hydrogen, R⁸ is (C₂-C₄)alkynyl, R⁹ is halo, and 10° is hydroxyl, wherein the remaining variables are as described above for formula Ia, Ia′, or Ib or any one of the fourth or sixth to eighth embodiments.

As part of a tenth embodiment, R³ in the compounds of structural formula Ia, Ia′, or Ib, or a pharmaceutically acceptable salt thereof, is

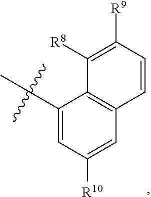

wherein the remaining variables are as described above for formula Ia, Ia′, or Ib or any one of the fourth or sixth to ninth embodiments. Alternatively, as part of a tenth embodiment, R³ in the compounds of structural formula Ia, Ia′, or Ib, or a pharmaceutically acceptable salt thereof, is

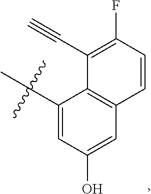 [SMILES: *c1cc(O)cc2ccc(F)c(C#C)c12]

wherein the remaining variables are as described above for formula Ia, Ia′, or Ib or any one of the fourth or sixth to ninth embodiments.

As part of an eleventh embodiment, R³ in the compounds of structural formula Ia, Ia′, or Ib, or a pharmaceutically acceptable salt thereof, is selected from

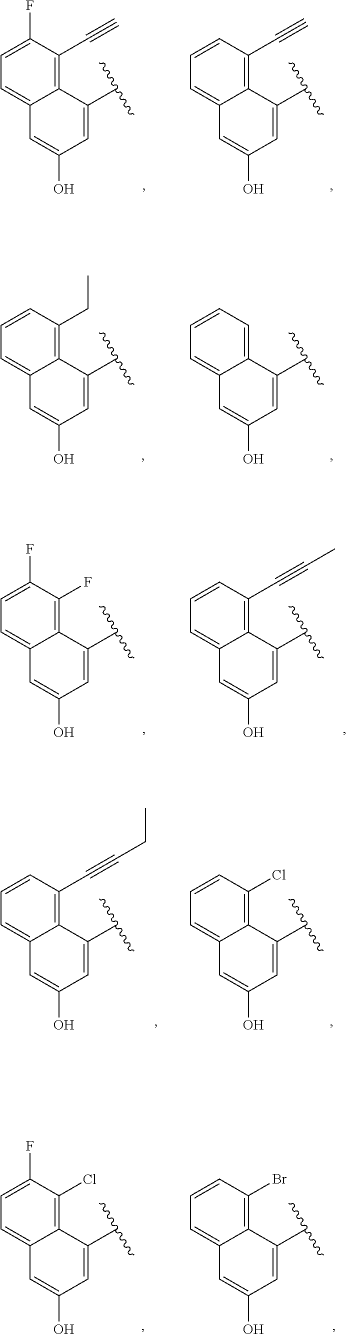

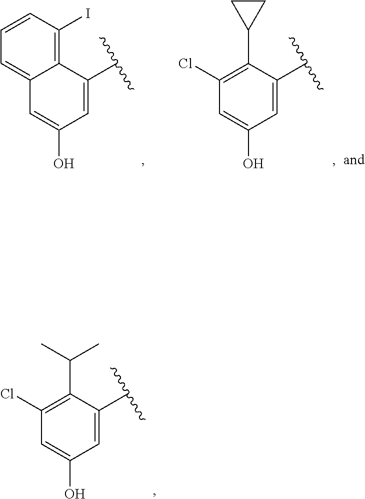 [SMILES: *c1cc(O)cc2cccc(I)c12 ; *c1cc(O)cc(Cl)c1C1CC1 ; *c1cc(O)cc(Cl)c1C(C)C]

wherein the remaining variables are as described above for formula Ia, Ia′, or Ib or any one of the fourth, sixth, or seventh embodiments.

As part of a twelfth embodiment, m in the compound of structural formula Ia′, or a pharmaceutically acceptable salt thereof, is 0 or 1, wherein the remaining variables are as described above for formula Ia, Ia′, or Ib or any one of the fourth or sixth to eleventh embodiments. Alternatively, as part of a twelfth embodiment, m in the compound of structural formula Ia′, or a pharmaceutically acceptable salt thereof, is 1, wherein the remaining variables are as described above for formula Ia, Ia′, or Ib or any one of the fourth or sixth to eleventh embodiments.

As part of a thirteenth embodiment, R⁵ in the compound of structural formula Ia′, or a pharmaceutically acceptable salt thereof, is halo, wherein the remaining variables are as described above for formula Ia, Ia′, or Ib or any one of the fourth or sixth to twelfth embodiments.

As part of a fourteenth embodiment, z in the compound of structural formula Ia′, or a pharmaceutically acceptable salt thereof, is 0 or 2, wherein the remaining variables are as described above for formula Ia, Ia′, or Ib or any one of the fourth or sixth to thirteenth embodiments. Alternatively, as part of a fourteenth embodiment, z in the compound of structural formula Ia′, or a pharmaceutically acceptable salt thereof, is 0, wherein the remaining variables are as described above for formula Ia, Ia′, or Ib or any one of the fourth or sixth to thirteenth embodiments.

As part of a fifteenth embodiment, le in the compound of structural formula Ia, Ia′, or Ib, or a pharmaceutically acceptable salt thereof, is

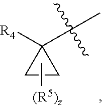

wherein the remaining variables are as described above for formula Ia, Ia′, or Ib or any one of the fourth or sixth to fourteenth embodiments. Alternatively, as part of a fifteenth embodiment, le in the compound of structural formula Ia, Ia′, or Ib is selected from

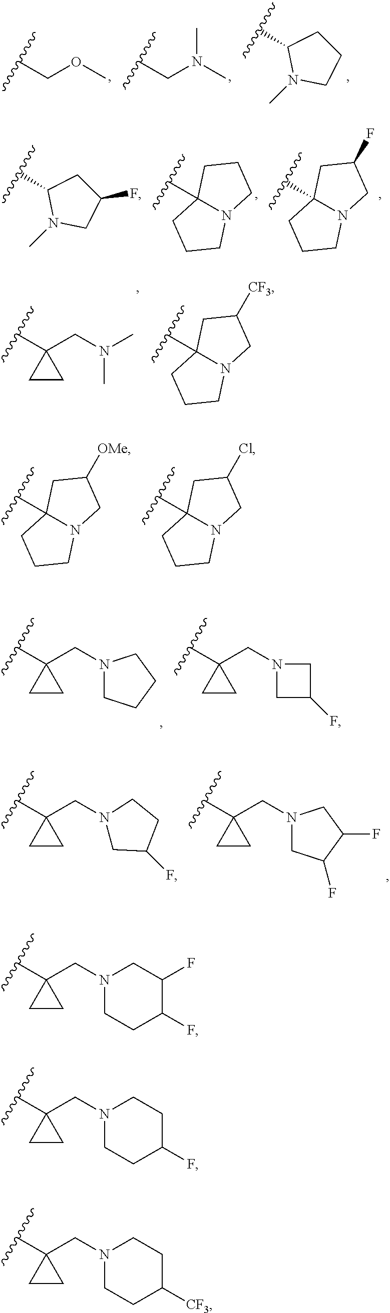

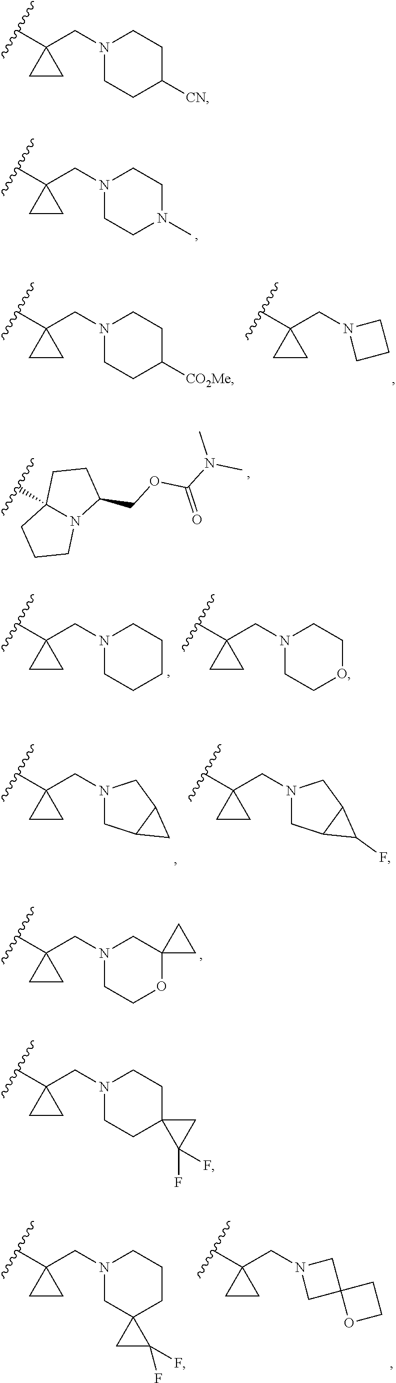

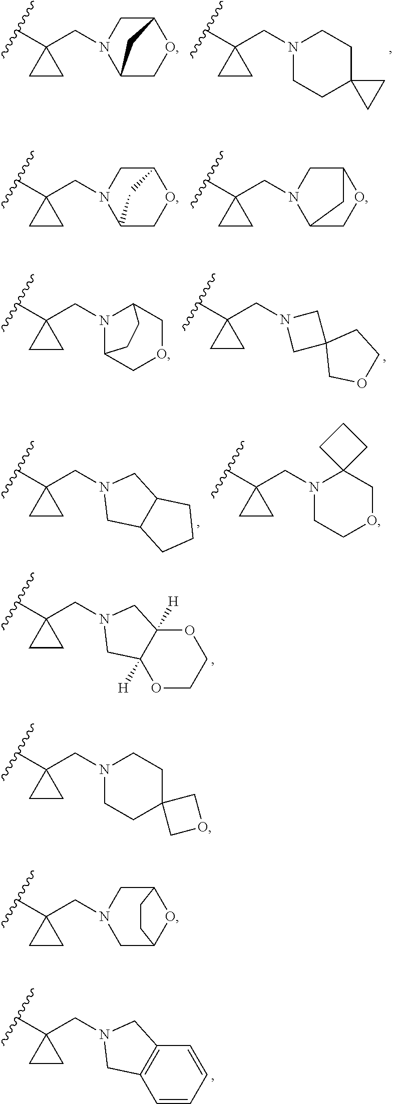

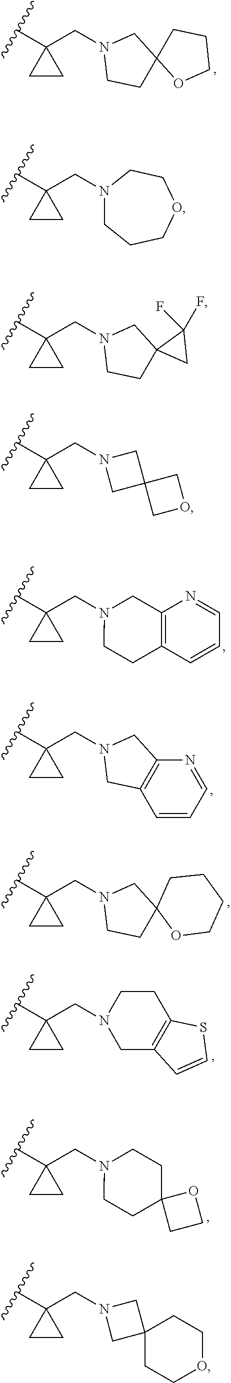

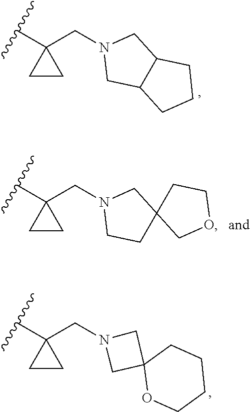 [SMILES: *C1(CN2CC3CCCC3C2)CC1 ; *C1(CN2CCC3(CCOC3)C2)CC1 ; "and" ; *C1(CN2CC3(CCCCO3)C2)CC1]

wherein the remaining variables are as described above for formula Ia, Ia′, or Ib or any one of the fourth or sixth to fourteenth embodiments.

As part of a sixteenth embodiment, R⁴ in the compounds and pharmaceutically acceptable salts described herein is -(C₁-C₄)alkylheterocyclyl optionally substituted with 1 to 3 groups selected from It', wherein the remaining variables are as described above for formula Ia, Ia′, or Ib or any one of the fourth or sixth to fifteenth embodiments.

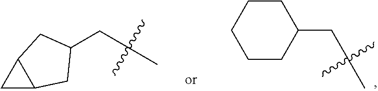 [SMILES: *CC1CC2CC2C1 ; *CC1CCCCC1]

Alternatively, as part of a sixteenth embodiment, R⁴ is each optionally substituted with 1 to 3 groups selected from R^{c}, wherein the remaining variables are as described above for formula Ia, Ia′, or Ib or any one of the fourth or sixth to fifteeenth embodiments.

As part of a seventeenth embodiment, RC in the compound of structural formula Ia, Ia′, or Ib, or a pharmaceutically acceptable salt thereof, is selected from (C₁-C₄)alkyl, (C₁-C₄)haloalkyl, and halo, wherein the remaining variables are as described above for formula Ia, Ia′, or Ib or any one of the fourth or sixth to sixteenth embodiments. Alternatively, as part of a seventeenth embodiment, RC in the compound of structural formula Ia, Ia′, or Ib, or a pharmaceutically acceptable salt thereof, is selected from (C₁-C₄)haloalkyl and halo, wherein the remaining variables are as described above for formula Ia, Ia′, or Ib or any one of the fourth or sixth to sixteenth embodiments.

As part of an eighteenth embodiment, R² in the compound of structural formula Ia or Ia′ is selected from hydrogen, (C₁-C₄)alkyl, (C₁-C₄)haloalkyl, (C₁-C₄)alkoxy, (C₁-C₄)haloalkoxy, (C₁-C₄)alkynyl, (C₁-C₄)alkenyl, halo, (C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein the remaining variables are as described above for formula Ia or Ia′ or any one of the fourth or sixth to seventeenth embodiments. Alternatively, as part of an eighteenth embodiment, R² in the compound of structural formula Ia or Ia′, or a pharmaceutically acceptable salt thereof, is selected from hydrogen and (C₁-C₄)alkoxy, wherein the remaining variables are as described above for formula Ia or Ia′ or any one of the fourth or sixth to seventeenth embodiments. In another alternative, as part of an eighteenth embodiment, R² in the compound of structural formula Ia or Ia′, or a pharmaceutically acceptable salt thereof, is

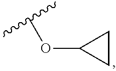 [SMILES: *OC1CC1]

selected from hydrogen, chloro, cyano, methoxy, ethoxy, CF₃, OCF₃, and wherein the remaining variables are as described above for formula Ia or Ia′ or any one of the fourth or sixth to seventeenth embodiments.

Additional compounds are further disclosed in the Exemplification and are included in the present disclosure. Pharmaceutically acceptable salts thereof as well as the neutral forms are included.

### 4. Uses, Formulation and Administration

Compounds and compositions described herein are generally useful as anticancer therapies. In one aspect, the disclosed compounds and compositions behave as inhibitors of KRAS(G12D). Their mechanisms of action include, but are not limited to, inhibiting KRAS(G12D) and thereby impeding down-stream signals that may result in inhibition of cancer cell growth and/or induction of cancer cell death or other KRAS or KRAS(G12D) functions. In one aspect, the disclosed compounds effectuate the inhibition of KRAS(G12D).

Thus, provided herein are methods of treating conditions which are responsive to the inhibition of KRAS(G12D) comprising administering to a subject in need thereof, a therapeutically effective amount of one or more compounds or compositions described herein. Also provided is the use of one or more compounds or compositions described herein in the manufacture of a medicament for treating conditions which are responsive to the inhibition of KRAS(G12D). Further provided is the use of a compound or composition described herein for treating conditions which are responsive to the inhibition of KRAS(G12D).

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Chemical Synthesis

The representative examples that follow are intended to help illustrate the present disclosure, and are not intended to, nor should they be construed to, limit the scope of the invention. General starting materials used were obtained from commercial sources or prepared in other examples, unless otherwise noted.

### Preparation of Compounds

The compounds claimed herein were prepared following the procedures outlined in the following protocols.

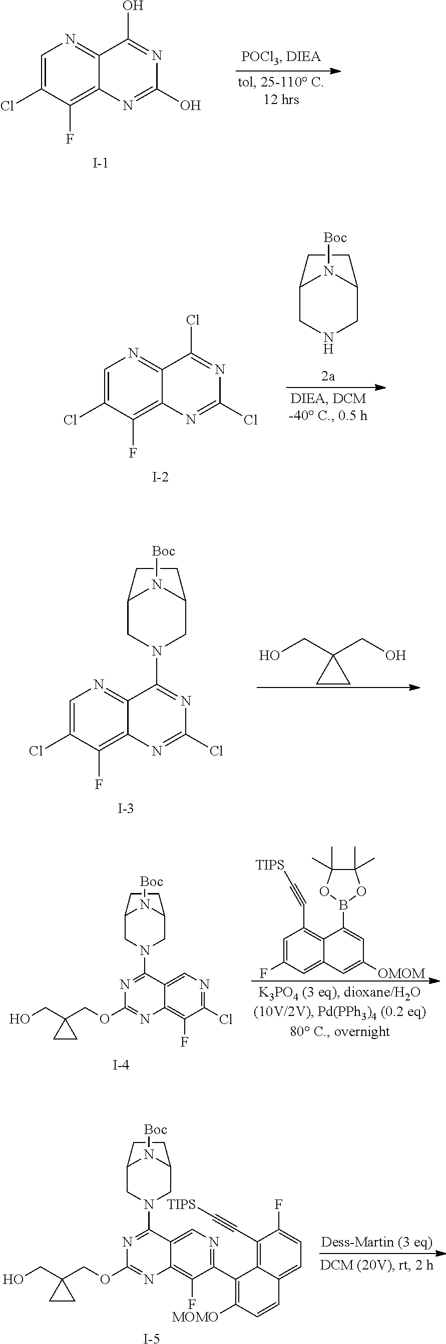

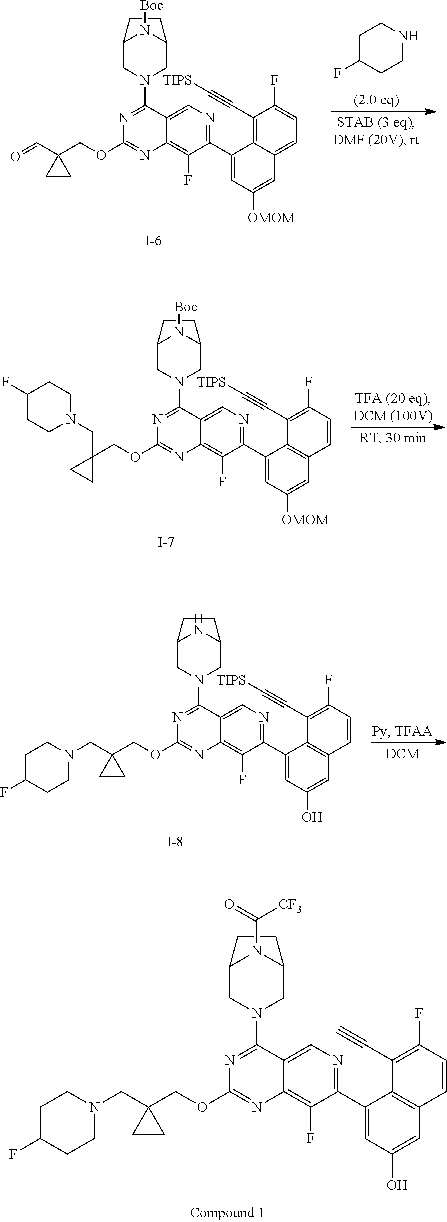

### Intermediate 2

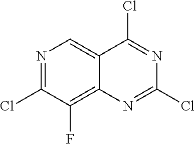 [SMILES: Fc1c(Cl)ncc2c(Cl)nc(Cl)nc12]

2,4,7-trichloro-8-fluoropyrido[4,3-d]pyrimidine. 2 batches: To a mixture of Intermediate 1 (50.0 g, 232 mmol, 1.00 eq) in Tol. (150 mL) was added POCl₃ (178 g, 1.16 mol, 108 mL, 5.00 eq) at 25° C. Then DIEA (65.9 g, 510 mmol, 88.9 mL, 2.20 eq) was added into the mixture blow 40° C. The mixture was stirred at 110° C. for 12 hrs. LC-MS showed desired MS was detected. The reaction mixture was distilled under reduced pressure to remove POCl₃ at 90° C. The residue was poured into Sat·NaHCO₃ slowly (keep pH=8). During this period, yellow precipitate was formed. It was collected by filtration and washed by H2O. The solid was used for next step directly. Intermediate 2 (101 g, 400 mmol, 86.2% yield) was obtained as brown solid. HNIVIR (DMSO-d₆, 400 MHz): δ8.92-8.86 (m, 1H). LC-MS: m/z 253.9 [M+H]⁺.

### Intermediate 3

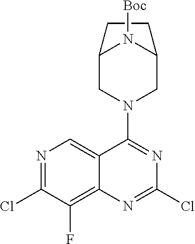 [SMILES: CC(C)(C)OC(=O)N1C2CCC1CN(c1nc(Cl)nc3c(F)c(Cl)ncc13)C2]

tert-butyl 3-(2,7-dichloro-8-fluoropyrido[4,3-d]pyrimidin-4-yl)-3,8-diazabicyclo[3.2.1]octane-8-carboxylate. 2 batches: To a mixture of Intermediate 2 (48.5 g, 192 mmol, 1.00 eq) in DCM (485 mL) was added a solution of compound 2a (38.7 g, 183 mmol, 0.950 eq) in DCM (120 mL). Then DIEA (49.7 g, 384 mmol, 66.9 mL, 2.00 eq) was added into the mixture at —40° C. and stirred at —40° C. for 0.5 hr under N₂. LCMS showed compound 2 was consumed, and desired MS was detected. The mixture was quenched by HCl (0.5 M) and the pH of the aqueous phase was acidified to 6˜7, then separated, the organic layer was dried over Na₂SO₄. The residue was purified by column chromatography (SiO2, TLC: Petroleum ether : Ethyl acetate =3: 1, R_{f}=0.4, Petroleum ether : Ethyl acetate =10: 1 to 1: 1, R_{f}=0.4). Intermediate 3(120 g, 280 mmol, 72.9% yield) was obtained as white solid. HNMR (DMSO-d₆, 400 MHz): 6 9.13-8.98 (m, 1H), 4.67-4.36 (m, 2H), 4.35-4.21 (m, 2H), 3.87-3.50 (m, 2H), 1.85-1.71 (m, 2H), 1.66-1.56 (m, 2H), 1.46 (s, 9H). LC-MS: m/z 428.0[M+H]⁺.

### Intermediate 4

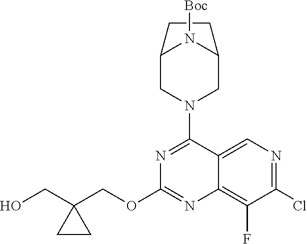 [SMILES: CC(C)(C)OC(=O)N1C2CCC1CN(c1nc(OCC3(CO)CC3)nc3c(F)c(Cl)ncc13)C2]

tert-butyl 3-(7-chloro-8-fluoro-24(1-(hydroxymethyl)cyclopropyl)methoxy)pyrido[4,3-d]pyrimidin-4-yl)-3,8-diazabicyclo[3.2.1]octane-8-carboxylate. To a stirred mixture of [1-(hydroxymethyl)cyclopropyl]methanol (14.31 g, 140 mmol, 3 eq) and t-BuONa (13.46 g, 140 mmol, 3 eq) in THF was added tert-butyl 3-{2,7-dichloro-8-fluoropyrido[4,3-d] pyrimidin-4-yl}-3,8-diazabicyclo[3.2.1]octane-8-carboxylate (20 g, 46.697 mmol, 1 eq) in portions at 0° C. under nitrogen atmosphere. The resulting mixture was stirred for lh under nitrogen atmosphere. The reaction was quenched with Water at 0° C. The resulting mixture was extracted with CH₂Cl₂ (3×10mL). The combined organic layers were washed with sat. NaCl aq. (2×5 mL), dried over anhydrous Na₂SO₄. The resulting mixture was concentrated under reduced pressure. The residue was purified by silica gel column chromatography, eluted with PE/ A (1:1) to afford intermediate 4 (14.5 g, 62.86%) as a white solid. LCMS (ES, m/z): 494 [M+H]+

### Intermediate 5

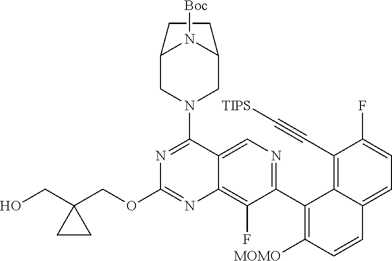

tert-butyl 3-(8-fluoro-7-(7-fluoro-2-(methoxymethoxy)-8-((triisopropylsilyl)ethy nyl)naphthalen-l-yl)-2-01-(hydroxymethyl)cyclopropyl)methoxy)pyrido[4,3-d]pyrimi din-4-yl)-3,8-diazabicyclo[3.2.1]octane-8-carboxylate. A solution of intermediate 4 (3 g, 6.073 mmol, 1.0 eq) and ((3 -fluoro-6-(m ethoxym ethoxy)-8-(4,4, 5,5 -tetram ethyl-1,3 ,2-dioxa borolan-2-yl)naphthalen-1-yl)ethynyl)triisopropylsilane (2.63 g, 9.110 mmol, 1.5 eq) in 1,4-dioxane(40 mL) was treated with Pd(PPh₃)₄ (1.40 g, 1.215 mmol, 0.2 eq) and K3PO4 (3.87 g, 18.219 mmol, 3.0 eq) under nitrogen atmosphere. The resulting mixture was stirred for ove might at 80° C. under nitrogen atmosphere. The reaction was quenched with Water at room t emperature. The resulting mixture was extracted with CH2C12 (3×10mL). The combined or ganic layers were washed with sat. NaCl aq. (2×5 mL), dried over anhydrous Na₂SO₄. The r esulting mixture was concentrated under reduced pressure. The residue was purified by silic a gel column chromatography, eluted with CH₂Cl₂/MeOH (10:1) to afford intermediate 5 (2.5 g, 66.38%) as a yellow solid. LCMS (ES, m/z): 620 [M+H]⁺

### Intermediate 6

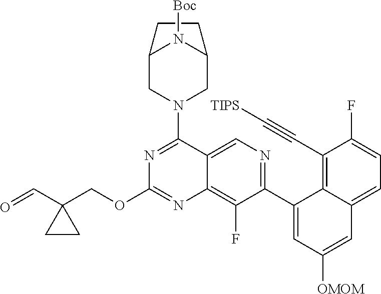

tert-butyl 3-(8-fluoro-7-(7-fluoro-3-(methoxymethoxy)-8-((triisopropylsilyl)ethynyl)naphthalen-l-yl)-2-((1-formylcyclopropyl)methoxy)pyrido[4,3-d]pyrimidin-4-yl)- 3,8-diazabicyclo[3.2.1]octane-8-carboxylate. To a stirred solution of intermediate 5 (200 mg, 0.323 mmol, 1 eq) in DCM was added Dess-Martin (410.38 mg, 0.969 mmol, 3 eq) in portions at room temperature. The resulting mixture was stirred for 2h at room temperature. The reaction was quenched with sat. NaHCO₃ (aq.) at 0° C. The resulting mixture was extracted with CH₂Cl₂ (3×10mL). The combined organic layers were washed with sat. NaCl (aq.) (3×5 mL), dried over anhydrous Na₂SO₄. After filtration, the filtrate was concentrated under reduced pressure. The crude product was used in the next step directly without further purification. LCMS (ES, m/z): 618 [M+H]⁺

### Intermediate 7

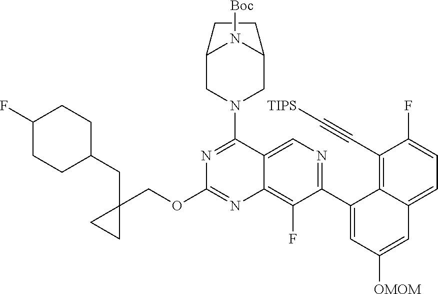

tert-butyl 3-(8-fluoro-7-(7-fluoro-3-(methoxymethoxy)-8-((triisopropylsilyl)ethy nyl)naphthalen-l-yl)-2-01-((4-fluoropiperidin-l-yl)methyl)cyclopropyl)methoxy)pyrid o [4,3-d] pyrimidin-4-yl)-3,8-diazabicyclo 13.2.11octane-8-carboxylate. To a stirred solutio n of intermediate 6 (100 mg, 0.162 mmol, 1 eq) and 4-fluoropiperidine hydrochloride (45.17 20 mg, 0.324 mmol, 2 eq) in DMF were added STAB (102.87 mg, 0.486 mmol, 3 eq) in portio ns at room temperature. The resulting mixture was stirred for 16 h at room temperature. The reaction was quenched with Water at room temperature. The resulting mixture was extracte d with EtOAc (3×10mL). The combined organic layers were washed with sat. NaCl aq. (2x 5 mL), dried over anhydrous Na₂SO₄. After filtration, the filtrate is concentrated under redu ced pressure. The crude product was used in the next step directly without further purificatio n. LCMS (ES, m/z): 605 [M+H]⁺

### Intermediate 8

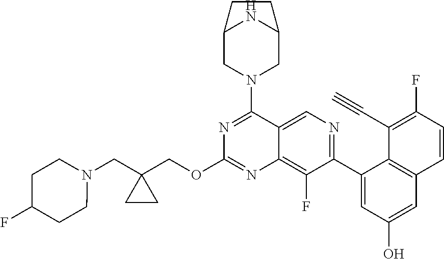 [SMILES: C#Cc1c(F)ccc2cc(O)cc(-c3ncc4c(N5CC6CCC(C5)N6)nc(OCC5(CN6CCC(F)CC6)CC5)nc4c3F)c12]

4-(4-(3,8-diazabicyclo[3.2.1]octan-3-yl)-8-fluoro-2-01-((4-fluoropiperidin-l-y1)methyl)cyclopropyl)methoxy)pyrido[4,3-d]pyrimidin-7-yl)-5-ethynyl-6-fluoronaphthalen-2-ol. To a stirred solution of intermediate 7 (80 mg, 0.132 mmol, 1.0 eq) in DCM was added TFA (1 mL, 13.463 mmol, 101.84 eq) dropwise at room temperature.

The resulting mixture was stirred for lh at room temperature. The resulting mixture was concentrated under reduced pressure. The crude product was purified by Prep-HPLC with the following conditions (Column, XBridge Prep OBD C18 Column, 30×150 mm, 5 μm; mobile phase, Water(10 mmol/L NH4HCO3) and ACN (30% ACN up to 80% in 10 min); Detector, uv 220 nm).This resulted in Intermediate 8 (38.15 mg, 47.69%) as a white solid. ¹H NMR (DMSO-d₆, 400 MHz): 6 10.15 (s, 1H), 9.03 (s, 1H), 7.97 (dd, J=9.2, 6.0 Hz, 1H), 7.46 (t, J=9.2 Hz, 1H), 7.39 (d, J=2.4 Hz, 1H), 7.17 (d, J=2.4 Hz, 1H), 4.69 (dt, J=7.6, 3.6 Hz, 1H), 4.48 (d, J=12.0 Hz, 1H), 4.34-4.22 (m, 3H), 3.93 (d, J=1.2 Hz, 1H), 3.67-3.59 (m, 1H), 3.54 (t, J=5.6 Hz, 3H), 2.74 (s, 1H), 2.56 (s, 2H), 2.30 (t, J=7.6 Hz, 20 4H), 1.88-1.76 (m, 2H), 1.66 (s, 6H), 0.64 (q, J=3.2 Hz, 2H), 0.40 (t, J=3.2 Hz, 2H). LC-MS: m/z 628.9 [M+H]⁺.

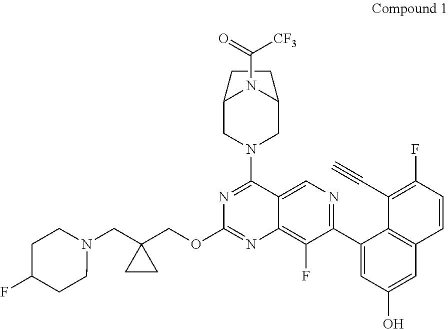 [SMILES: C#Cc1c(F)ccc2cc(O)cc(-c3ncc4c(N5CC6CCC(C5)N6C(=O)C(F)(F)F)nc(OCC5(CN6CCC(F)CC6)CC5)nc4c3F)c12]

1-(3-(7-(8-ethynyl-7-fluoro-3-hydroxynaphthalen-1-yl)-8-fluoro-24(14(4-fluoropi peridin-l-yl)methyl)cyclopropyl)methoxy)pyrido[4,3-d]pyrimidin-4-yl)-3,8-diazabicycl o[3.2.1]octan-8-yl)-2,2,2-trifluoroethan-1-one. To a solution of compound 3 (1.00 eq) in DCM (2 mL) was added Py (5.00 eq) and TFAA (1.50 eq) at 0° C., then the mixture was stir red at 25° C. for 2 hrs. LC-MS showed Reactant 1 was consumed completely and one main p eak with desired mass was detected. The reaction mixture was quenched by addition H₂O (5 mL) at 10° C., and extracted with DCM (5 mL*2). The combined organic layers were was hed with brine (5 mL), dried over Na2SO4, filtered and concentrated under reduced pressure to give a residue. The residue was purified by prep-HPLC (FA condition; column: Phenome nex lunaC18 150*25mm* 10um;mobile phase: [water(FA)-ACN];B%: 18%-48%,10min).

Compound 1 (20.0 mg, 25.4 !Amok 47.1% yield, 93.4% purity, HCl ) was obtained as a yell ow solid. ¹H NMR (DMSO-d₆, 400 MHz): δ10.16 (s, 1H), 9.05 (d, J=1.2 Hz, 1H), 7.98 (d d, J=9.2, 6.0 Hz, 1H), 7.47 (t, J=9.0 Hz, 1H), 7.40 (d, J=2.4 Hz, 1H), 7.18 (t, J=2.4 Hz, 1H), 4.80 (s, 1H), 4.73 (d, J=12.4 Hz, 1H), 4.65 (s, 2H), 4.59 — 4.48 (m, 1H), 4.35 — 4.24 ( m, 2H), 3.94 (dd, J=6.4, 1.2 Hz, 1H), 3.76 (d, J=12.4 Hz, 1H), 3.68 (d, J=12.4 Hz, 1H), 2.48 (s, 1H), 2.31-2.29 (m, 4H), 1.90 — 1.75 (m, 6H), 1.65 (s, 2H), 0.65 (d, J=4.8 Hz, 2H), 0.45-0.40 (m, 2H). LC-MS: m/z 724.9 [M+H]⁺.

The following compounds were prepared according the general procedures described above using the appropriate starting materials.

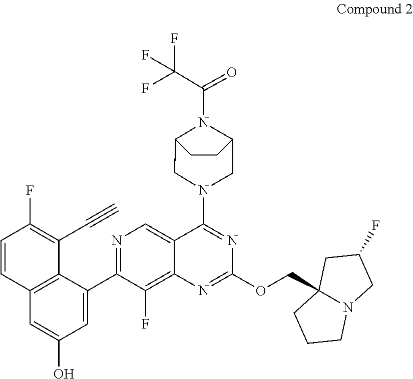 [SMILES: C#Cc1c(F)ccc2cc(O)cc(-c3ncc4c(N5CC6CCC(C5)N6C(=O)C(F)(F)F)nc(OC[C@]56CCCN5C[C@@H](F)C6)nc4c3F)c12]

1-(3-(7-(8-ethynyl-7-fluoro-3-hydroxynaphthalen-1-yl)-8-fluoro-2-(42S,7aR)-2-fluorotetrahydro-1H-pyrrolizin-7a(5H)-yHmethoxy)pyrido[4,3-d]pyrimidin-4-yl)-3,8-diazabicyclo[3.2.1]octan-8-yl)-2,2,2-trifluoroethan-1-one ¹HNMR (400 MHz, DMSO-d6) δ11.03-10.79 (s, 1H), 10.24 (s, 1H), 9.12 (s, 1H), 8.00 (dd, J=5.9, 9.3 Hz, 1H), 7.48 (t, J=9.0 Hz, 1H), 7.42 (d, J=2.6 Hz, 1H), 7.20 (s, 1H), 5.72- 5.38 (m, 1H), 4.84-4.57 (m, 6H), 3.92 (dd, J=3.5, 5.3 Hz, 1H),3.86-3.67 (m, 4H), 3.54 (s, 2H), 2.20-1.84 (m, 10H), LC-MS: m/z 697.3 [M+H]⁺.

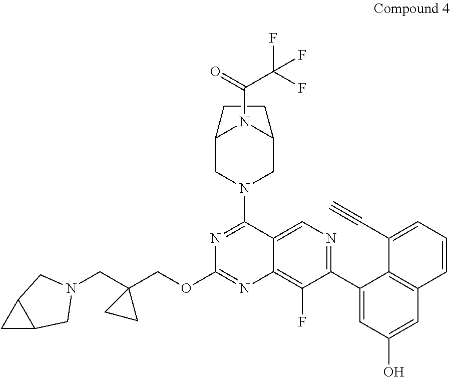 [SMILES: C#Cc1cccc2cc(O)cc(-c3ncc4c(N5CC6CCC(C5)N6C(=O)C(F)(F)F)nc(OCC5(CN6CC7CC7C6)CC5)nc4c3F)c12]

1-(3-{2-1(1-{3-azabicyclo[3.1.0]hexan-3-ylmethyl}cyclopropyl)methoxyl-7-(8-eth ynyl-7-fluoro-3-hydroxynaphthalen-1-yl)-8-fluoropyrido[4,3-d]pyrimidin-4-yl}-3,8-diazabicyclo[3.2.1]octan-8-yl)-2,2,2-trifluoroethanone. 1H NMR (DMSO-d6, 400 MHz): δ10.15 (s, 1H), 9.05 (s, 1H), 7.98 (dd, J=9.2, 6.0 Hz, 1H), 7.47 (t, J=9.2 Hz, 1H), 7.40 (d, J=2.4 Hz, 1H), 7.18 (t, J=2.4 Hz, 1H), 4.80 (s, 1H), 4.72 (d, J=13.6 Hz, 1H), 4.65 (s, 1H), 4.52 (d, J=12.8 Hz, 1H), 4.33-4.18 (m, 2H), 3.92 (dd, J=6.8, 1.2 Hz, 1H), 3.76 (d, J =12.8 Hz, 1H), 3.68 (d, J=12.8 Hz, 1H), 2.96 (dd, J=8.4, 6.2 Hz, 2H), 2.46-2.30 (m, 2H), 2.23 (d, J=8.4 Hz, 2H), 2.05-1.88 (m, 4H), 1.32 (dd, J=9.6, 5.2 Hz, 2H), 0.59 (s, 2H), 0.55-0.50 (m, 1H), 0.40 (s, 2H), 0.25 (p, J=3.6 Hz, 1H). LC-MS: m/z 705.0 [M+H]+.

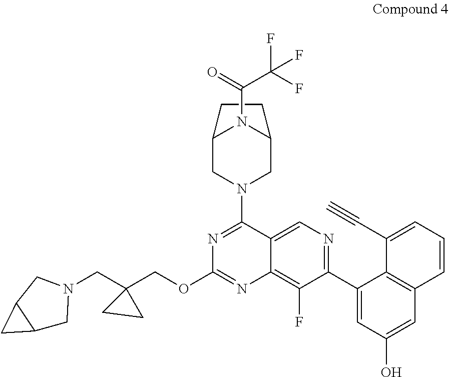 [SMILES: C#Cc1cccc2cc(O)cc(-c3ncc4c(N5CC6CCC(C5)N6C(=O)C(F)(F)F)nc(OCC5(CN6CC7CC7C6)CC5)nc4c3F)c12]

1-(3-(2-((1-((3-azabicyclo[3.1.0]hexan-3-yl)methyl)cyclopropyl)methoxy)-7-(8-ethynyl-3-hydroxynaphthalen-1-yl)-8-fluoropyrido[4,3-d]pyrimidin-4-yl)-3,8-diazabicyclo[3.2.1]octan-8-yl)-2,2,2-trifluoroethan-1-one. ¹H NMR (DMSO-d₆, 400 MHz): δ10.14 (s, 1H), 9.04 (s, 1H), 7.89 (dd, J=8.0, 1.6 Hz, 1H), 7.46-7.38 (m, 2H), 7.35 (d, J=2.4 Hz, 1H), 7.12 (t, J=2.4 Hz, 1H), 4.80 (s, 1H), 4.73 (dd, J=12.4, 5.6 Hz, 1H), 4.64 (s, 1H), 4.52 (d, J=12.8 Hz, 1H), 4.27-4.17 (m, 2H), 3.76 (d, J=12.8 Hz, 1H), 3.68 (d, J=12.8 Hz, 1H), 3.58 (d, J=6.4 Hz, 1H), 3.00-2.91 (m, 2H), 2.42 -2.35 (m, 2H), 2.23 (d, J=8.4 Hz, 2H), 2.00-1.86 (m, 3H), 1.31 (s, 2H), 0.56-0.52(m, 1H), 0.46 (d, J=4.0 Hz, 3H), 0.40 (s, 2H), 0.25 (s, 1H). LC-MS: m/z 687.1 [M+H]t

### Prophetic Compounds

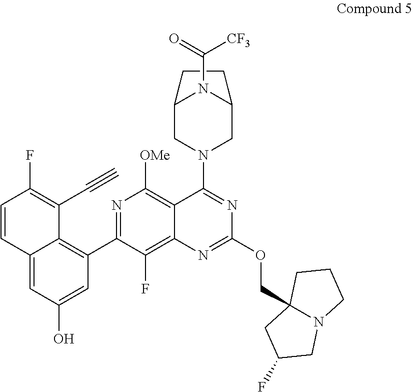 [SMILES: C#Cc1c(F)ccc2cc(O)cc(-c3nc(OC)c4c(N5CC6CCC(C5)N6C(=O)C(F)(F)F)nc(OC[C@@]56CCCN5C[C@H](F)C6)nc4c3F)c12]

1-(3-(7-(8-ethynyl-7-fluoro-3-hydroxynaphthalen-l-yl)-8-fluoro-2-(((2R,7aS)-2-fluorotetrahydro-1H-pyrrolizin-7a(5H)-yl)methoxy)-5-methoxypyrido[4,3-d]pyrimidin-4-y1)-3,8-diazabicyclo[3.2.1]octan-8-yl)-2,2,2-trifluoroethan-l-one

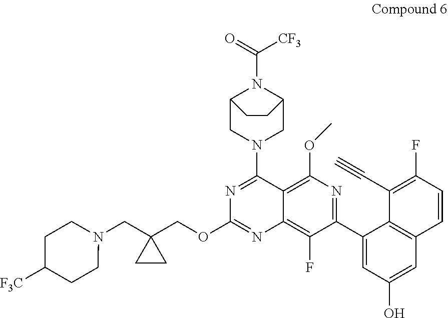 [SMILES: C#Cc1c(F)ccc2cc(O)cc(-c3nc(OC)c4c(N5CC6CCC(C5)N6C(=O)C(F)(F)F)nc(OCC5(CN6CCC(C(F)(F)F)CC6)CC5)nc4c3F)c12]

1-((1R,5 S)--(7-(8-ethynyl-7-fluoro-3 -hydroxynaphthal en-1-yl)-8 -fluoro-5-m ethoxy-24(1-((4-(trifluorom ethyl)pip eridin-l-yl)m ethyl)cycl opropyl)m ethoxy)pyri do [4,3 -d] pyrimi din-4-y1)-3,8-diazabicyclo[3.2.1] octan-8-yl)-2,2,2-trifluoroethan-l-one1-((1R,5 S)-3 -(7-(8-ethynyl-7-fluoro-3 -hydroxynaphthal en-1-yl)-8-fluoro-5-methoxy-2-((1-((4-(trifluorom ethyl)piperidin-l-yl)methyl)cyclopropyl)methoxy)pyrido [4,3 -d] pyrimi din-4-yl)-3, 8-diazabicyclo[3.2.1] octan-8-yl)-2,2,2-trifluoroethan-l-one

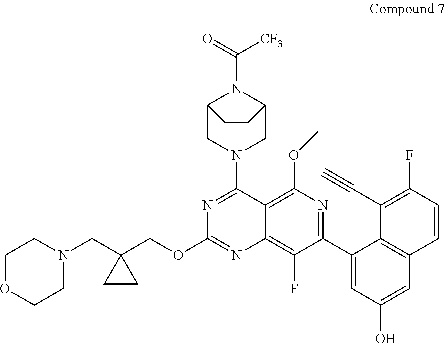 [SMILES: C#Cc1c(F)ccc2cc(O)cc(-c3nc(OC)c4c(N5CC6CCC(C5)N6C(=O)C(F)(F)F)nc(OCC5(CN6CCOCC6)CC5)nc4c3F)c12]

1-((1R, 5 S)-3-(7-(8-ethynyl-7-fluoro-3-hydroxynaphthal en-l-yl)-8-fluoro-5-methoxy-2-((1-(morpholinomethyl)cycl opropyl)methoxy)pyrido[4,3 -d]pyrimidin-4-yl)-3,8-diazabicyclo[3.2.1] octan-8-yl)-2,2,2-trifluoroethan-1-one

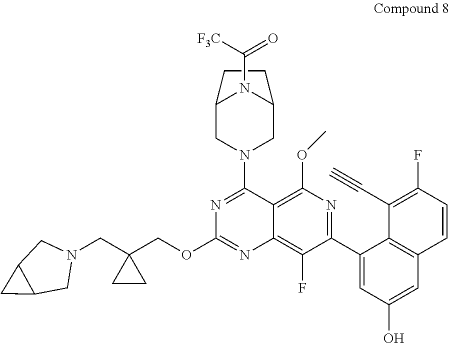 [SMILES: C#Cc1c(F)ccc2cc(O)cc(-c3nc(OC)c4c(N5CC6CCC(C5)N6C(=O)C(F)(F)F)nc(OCC5(CN6CC7CC7C6)CC5)nc4c3F)c12]

1-(3-(2-((1-((3 -azabicycl o [3.1. O]hexan-3 -yl)methyl)cycl opropyl)methoxy)-7-(8-ethynyl-7-fluoro-3 -hydroxynaphthal en-1-yl)-8-fluoro-5-methoxypyrido[4,3 -d]pyrimi din-4-yl)-3,8-diazabicyclo[3.2.1] octan-8-yl)-2,2,2-trifluoroethan-1-one

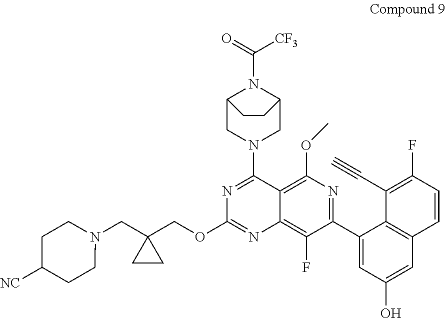 [SMILES: C#Cc1c(F)ccc2cc(O)cc(-c3nc(OC)c4c(N5CC6CCC(C5)N6C(=O)C(F)(F)F)nc(OCC5(CN6CCC(C#N)CC6)CC5)nc4c3F)c12]

1-((14(7-(8-ethynyl-7-fluoro-3 -hydroxynaphthal en-1-yl)-8-fluoro-5-methoxy-4-((1 R,5 S)-8-(2,2,2-trifluoroacetyl)-3,8-diazabicyclo[3.2.1] octan-3-yl)pyrido[4,3 -d]pyrimidin-2-yl)oxy)methyl)cyclopropyl)methyl)piperidine-4-carbonitrile

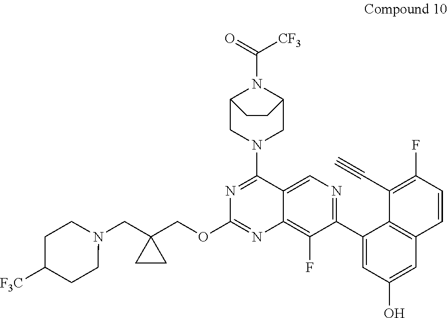 [SMILES: C#Cc1c(F)ccc2cc(O)cc(-c3ncc4c(N5CC6CCC(C5)N6C(=O)C(F)(F)F)nc(OCC5(CN6CCC(C(F)(F)F)CC6)CC5)nc4c3F)c12]

1-((1 R, 5 S)-3-(7-(8-ethynyl-7-fluoro-3-hydroxynaphthalen-1-yl)-8-fluoro-2-((1-((4-(trifluoromethyl)piperi din-l- yl)methyl)cyclopropyl)methoxy)pyrido [4,3-d]pyrimidin-4-yl)-3,8-diazabicyclo[3.2.1] octan-8-yl)-2,2,2-trifluoroethan-1-one

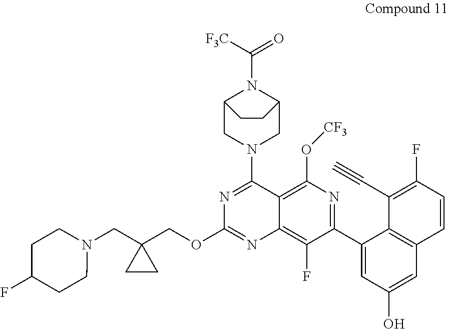 [SMILES: C#Cc1c(F)ccc2cc(O)cc(-c3nc(OC(F)(F)F)c4c(N5CC6CCC(C5)N6C(=O)C(F)(F)F)nc(OCC5(CN6CCC(F)CC6)CC5)nc4c3F)c12]

1-((1 R, 5 S)-3-(7-(8-ethynyl-7-fluoro-3-hydroxynaphthalen-1-yl)-8-fluoro-2-((1-((4-fluoropiperidi n-l-yl)methyl)cyclopropyl)methoxy)-5-(trifluoromethoxy)pyrido [4,3-d]pyrimidin-4-yl)-3,8-diazabicyclo[3.2.1] octan-8-yl)-2,2,2-trifluoroethan-1-one

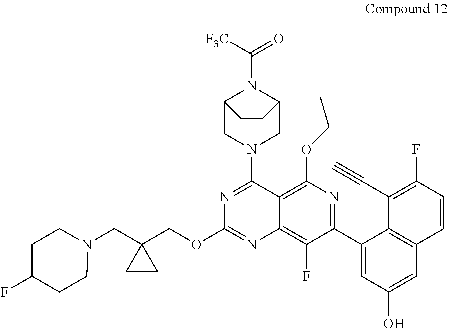 [SMILES: C#Cc1c(F)ccc2cc(O)cc(-c3nc(OCC)c4c(N5CC6CCC(C5)N6C(=O)C(F)(F)F)nc(OCC5(CN6CCC(F)CC6)CC5)nc4c3F)c12]

1-((1 R, 5 S)-3-(5-ethoxy-7-(8-ethynyl-7-fluoro-3-hydroxynaphthalen-l-yl)-8-fluoro-2-((1-((4-((4-fluoropiperidin-1-yl)methyl)cyclopropyl)methoxy)pyrido[4,3-d]pyrimidin-4-yl)-3,8-diazabicyclo[3.2.1] octan-8-yl)-2,2,2-trifluoroethan-1-one

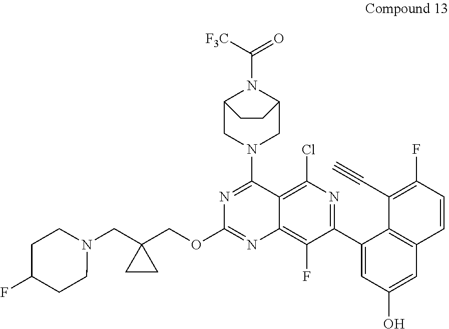 [SMILES: C#Cc1c(F)ccc2cc(O)cc(-c3nc(Cl)c4c(N5CC6CCC(C5)N6C(=O)C(F)(F)F)nc(OCC5(CN6CCC(F)CC6)CC5)nc4c3F)c12]

1-((1 R, 5 S)-3-(5-chloro-7-(8-ethynyl-7-fluoro-3-hydroxynaphthalen-1-yl)-8-fluoro-2-((1-((4-fluoropiperidin-1-yl)methyl)cyclopropyl)methoxy)pyrido[4,3-d]pyrimidin-4-yl)-3,8-diazabicyclo[3.2.1] octan-8-yl)-2,2,2-trifluoroethan-1-one

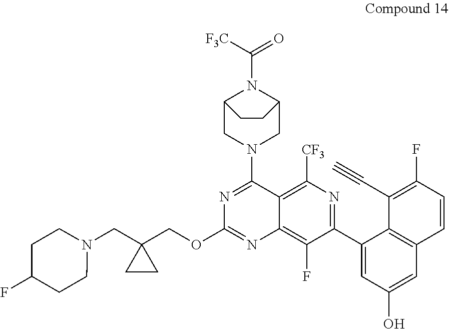 [SMILES: C#Cc1c(F)ccc2cc(O)cc(-c3nc(C(F)(F)F)c4c(N5CC6CCC(C5)N6C(=O)C(F)(F)F)nc(OCC5(CN6CCC(F)CC6)CC5)nc4c3F)c12]

1-((1 R, 5 S)-3-(7-(8-ethynyl-7-fluoro-3-hydroxynaphthalen-1-yl)-8-fluoro-2-((1-((4-fluoropiperidi n-l-yl)methyl)cycl opropyl)methoxy)-5-(trifluorom ethyppyri do [4,3-d]pyrimidin-4-yl)-3,8-diazabicyclo[3.2.1] octan-8-yl)-2,2,2-trifluoroethan-1-one

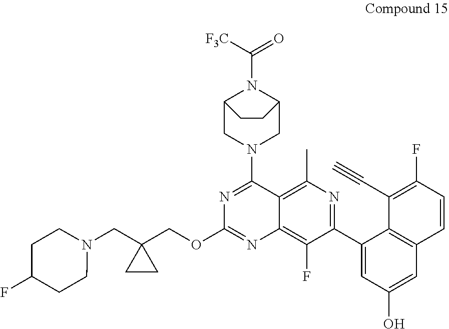 [SMILES: C#Cc1c(F)ccc2cc(O)cc(-c3nc(C)c4c(N5CC6CCC(C5)N6C(=O)C(F)(F)F)nc(OCC5(CN6CCC(F)CC6)CC5)nc4c3F)c12]

1-((1 R, 5 S)-3-(7-(8-ethynyl-7-fluoro-3-hydroxynaphthalen-1-yl)-8-fluoro-2-((1-((4-fluoropiperidin-l-yl)methyl)cyclopropyl)methoxy)-5- methylpyrido[4,3-d]pyrimidin-4-yl)-3,8-diazabicyclo[3.2.1] octan-8-yl)-2,2,2-trifluoroethan-1-one

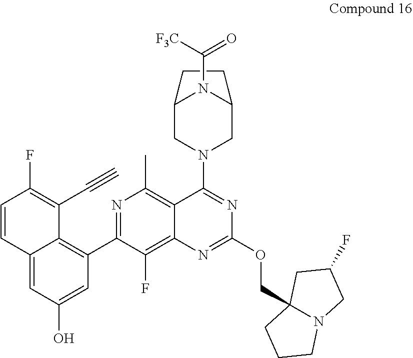 [SMILES: C#Cc1c(F)ccc2cc(O)cc(-c3nc(C)c4c(N5CC6CCC(C5)N6C(=O)C(F)(F)F)nc(OC[C@]56CCCN5C[C@@H](F)C6)nc4c3F)c12]

1-(3-(7-(8-ethynyl-7-fluoro-3-hydroxynaphthalen-l-yl)-8-fluoro-2-(((2S,7aR)-2-fluorotetrahydro-1H-pyrrolizin-7a(5H)-yl)methoxy)-5-methylpyrido[4,3-d]pyrimidin-4-yl)-5 3,8-diazabicyclo[3.2.1]octan-8-yl)-2,2,2-trifluoroethan-1-one

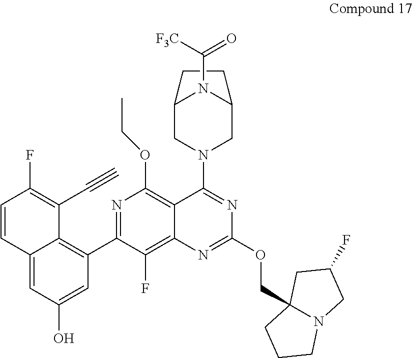 [SMILES: C#Cc1c(F)ccc2cc(O)cc(-c3nc(OCC)c4c(N5CC6CCC(C5)N6C(=O)C(F)(F)F)nc(OC[C@]56CCCN5C[C@@H](F)C6)nc4c3F)c12]

1-(3-(5-ethoxy-7-(8-ethynyl-7-fluoro-3-hydroxynaphthalen-l-yl)-8-fluoro-2-(((2S,7aR)-2-fluorotetrahydro-1H-pyrrolizin-7a(5H)-yl)methoxy)pyrido[4,3-d]pyrimidin-4-yl)-3,8-10 diazabicyclo[3.2.1]octan-8-yl)-2,2,2-trifluoroethan-1-one

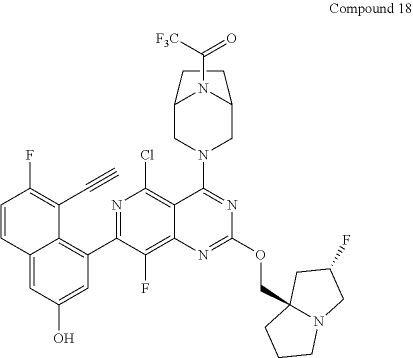 [SMILES: C#Cc1c(F)ccc2cc(O)cc(-c3nc(Cl)c4c(N5CC6CCC(C5)N6C(=O)C(F)(F)F)nc(OC[C@]56CCCN5C[C@@H](F)C6)nc4c3F)c12]

1-(3-(5-chloro-7-(8-ethynyl-7-fluoro-3-hydroxynaphthalen-l-yl)-8-fluoro-2-(((2S,7aR)-2-fluorotetrahydro-1H-pyrrolizin-7a(5H)-yl)methoxy)pyrido[4,3-d]pyrimidin-4-yl)-3,8-15 diazabicyclo[3.2.1]octan-8-yl)-2,2,2-trifluoroethan-1-one

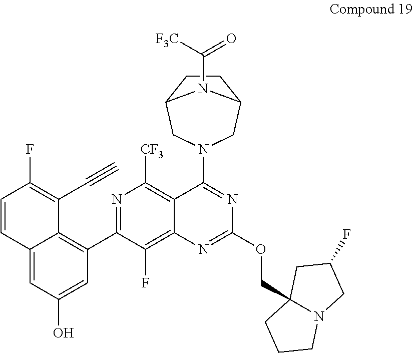 [SMILES: C#Cc1c(F)ccc2cc(O)cc(-c3nc(C(F)(F)F)c4c(N5CC6CCC(C5)N6C(=O)C(F)(F)F)nc(OC[C@]56CCCN5C[C@@H](F)C6)nc4c3F)c12]

1-(3-(7-(8-ethynyl-7-fluoro-3-hydroxynaphthalen-l-yl)-8-fluoro-2-(((2S,7aR)-2-fluorotetrahydro-1H-pyrrolizin-7a(5H)-yl)methoxy)-5-(trifluoromethyl)pyrido[4,3-d]pyrimidin-4-yl)-3,8-diazabicyclo[3.2.1]octan-8-yl)-2,2,2-trifluoroethan-1-one

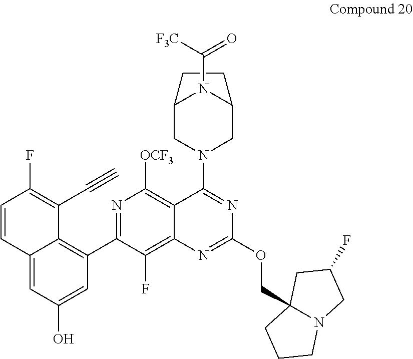 [SMILES: C#Cc1c(F)ccc2cc(O)cc(-c3nc(OC(F)(F)F)c4c(N5CC6CCC(C5)N6C(=O)C(F)(F)F)nc(OC[C@]56CCCN5C[C@@H](F)C6)nc4c3F)c12]

1-(3-(7-(8-ethynyl-7-fluoro-3-hydroxynaphthalen-l-yl)-8-fluoro-2-(((2S,7aR)-2-fluorotetrahydro-1H-pyrrolizin-7a(5H)-yl)methoxy)-5-(trifluoromethoxy)pyrido[4,3-d]pyrimidin-4-yl)-3,8-diazabicyclo[3.2.1]octan-8-yl)-2,2,2-trifluoroethan-1-one

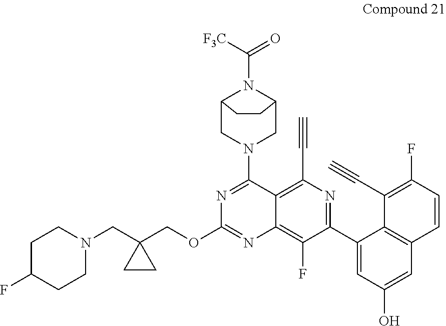 [SMILES: C#Cc1c(F)ccc2cc(O)cc(-c3nc(C#C)c4c(N5CC6CCC(C5)N6C(=O)C(F)(F)F)nc(OCC5(CN6CCC(F)CC6)CC5)nc4c3F)c12]

1-((1R,5S)-3-(5-ethynyl-7-(8-ethynyl-7-fluoro-3-hydroxynaphthalen-l-yl)-8-fluoro-2-((1-((4-fluoropiperidin-1-yl)methyl)cyclopropyl)methoxy)pyrido[4,3-d]pyrimidin-4-yl)-3,8-diazabicyclo[3.2.1]octan-8-yl)-2,2,2-trifluoroethan-1-one

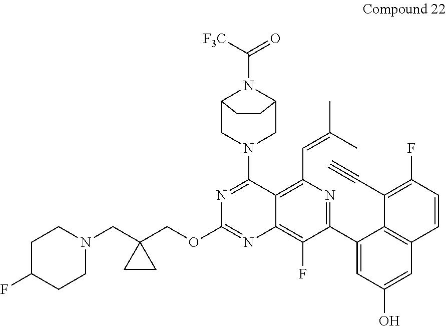 [SMILES: C#Cc1c(F)ccc2cc(O)cc(-c3nc(C=C(C)C)c4c(N5CC6CCC(C5)N6C(=O)C(F)(F)F)nc(OCC5(CN6CCC(F)CC6)CC5)nc4c3F)c12]

1-((1 R, 5 S)-3-(7-(8-ethynyl-7-fluoro-3-hydroxynaphthalen-1-yl)-8-fluoro-2-((1-((4-fluoropiperidi n-l-yl)methyl)cycl opropyl)methoxy)-5-(2-methylprop-1-en-l-yl)pyrido[4,3-d]pyrimidin-4-yl)-3,8-diazabicyclo[3.2.1] octan-8-yl)-2,2,2-trifluoroethan-l-one

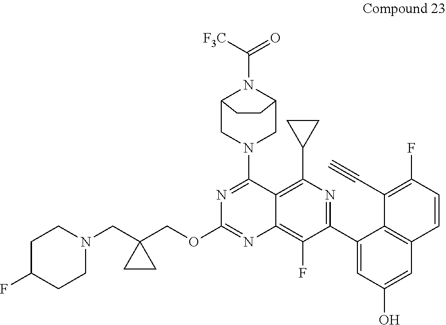 [SMILES: C#Cc1c(F)ccc2cc(O)cc(-c3nc(C4CC4)c4c(N5CC6CCC(C5)N6C(=O)C(F)(F)F)nc(OCC5(CN6CCC(F)CC6)CC5)nc4c3F)c12]

1-((1 R, 5 S)-3-(5-cyclopropyl -7-(8-ethynyl-7-fluoro-3-hydroxynaphthalen-1-yl)-8-fluoro-2-((1-((4-fluoropiperidin-l-yl)methyl)cyclopropyl)methoxy)pyrido[4,3-d]pyrimidin-4-yl)-3,8-diazabicyclo[3.2.1]octan-8-yl)-2,2,2-trifluoroethan-l-one

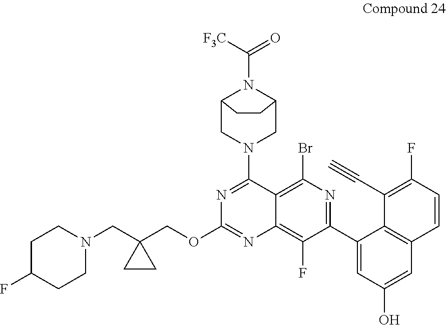 [SMILES: C#Cc1c(F)ccc2cc(O)cc(-c3nc(Br)c4c(N5CC6CCC(C5)N6C(=O)C(F)(F)F)nc(OCC5(CN6CCC(F)CC6)CC5)nc4c3F)c12]

1-((1 R, 5 S)-3-(5-bromo-7-(8-ethynyl-7-fluoro-3-hydroxynaphthalen-l-yl)-8-fluoro-241-((4-fluoropiperidin-1-yl)methyl)cyclopropyl)methoxy)pyrido[4,3-d]pyrimidin-4-yl)-3,8-diazabicyclo[3.2.1] octan-8-yl)-2,2,2-trifluoroethan-l-one

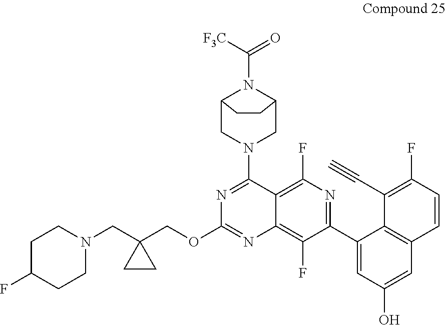 [SMILES: C#Cc1c(F)ccc2cc(O)cc(-c3nc(F)c4c(N5CC6CCC(C5)N6C(=O)C(F)(F)F)nc(OCC5(CN6CCC(F)CC6)CC5)nc4c3F)c12]

1-((1R,5S)-3-(7-(8-ethynyl-7-fluoro-3-hydroxynaphthalen-l-yl)-5,8-difluoro-2-((1-((4-fluoropiperidin-1-yl)methyl)cyclopropyl)methoxy)pyrido[4,3-d]pyrimidin-4-yl)-3,8-diazabicyclo[3.2.1] octan-8-yl)-2,2,2-trifluoroethan-1-one

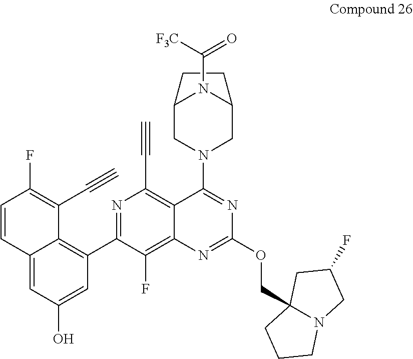 [SMILES: C#Cc1c(F)ccc2cc(O)cc(-c3nc(C#C)c4c(N5CC6CCC(C5)N6C(=O)C(F)(F)F)nc(OC[C@]56CCCN5C[C@@H](F)C6)nc4c3F)c12]

1-(3-(5 -ethynyl-7-(8-ethynyl-7-fluoro-3-hydroxynaphthalen-l-yl)-8-fluoro-2-(((2 S,7aR)-2-fluorotetrahydro-1H-pyrrolizin-7a(5H)-yl)methoxy)pyrido[4,3-d]pyrimidin-4-yl)-3,8-diazabicyclo[3.2.1] octan-8-yl)-2,2,2-trifluoroethan-1-one

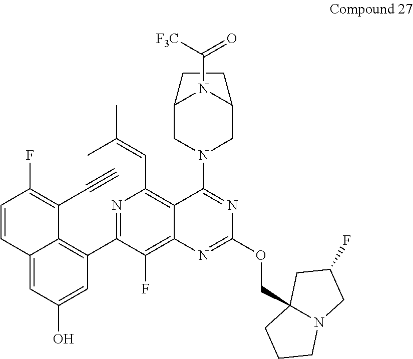 [SMILES: C#Cc1c(F)ccc2cc(O)cc(-c3nc(C=C(C)C)c4c(N5CC6CCC(C5)N6C(=O)C(F)(F)F)nc(OC[C@]56CCCN5C[C@@H](F)C6)nc4c3F)c12]

1-(3-(7-(8-ethynyl-7-fluoro-3-hydroxynaphthal en-1-yl)-8-fluoro-2-(((2 S,7aR)-2-fluorotetrahydro-1H-pyrrolizin-7a(5H)-yl)methoxy)-5-(2-methylprop-1-en-1-yl)pyrido[4,3-d]pyrimidin-4-yl)-3,8-diazabicyclo[3.2.1] octan-8-yl)-2,2,2-trifluoroethan-1 -one

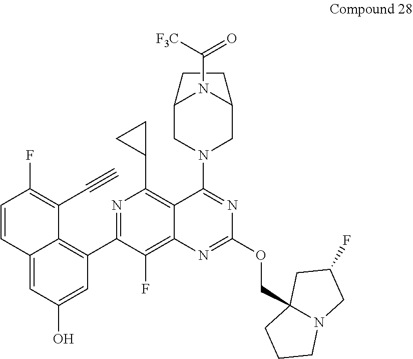 [SMILES: C#Cc1c(F)ccc2cc(O)cc(-c3nc(C4CC4)c4c(N5CC6CCC(C5)N6C(=O)C(F)(F)F)nc(OC[C@]56CCCN5C[C@@H](F)C6)nc4c3F)c12]

1-(3-(5 -cycl opropyl-7-(8-ethynyl-7-fluoro-3-hydroxynaphthal en-1-yl)-8-fluoro-2-(((2 S,7aR)-2-fluorotetrahydro-1H-pyrrolizin-7a(5H)-yl)methoxy)pyrido[4,3-d]pyrimidin-4-y1)-3,8-diazabicyclo[3.2.1] octan-8-yl)-2,2,2-trifluoroethan-1-one

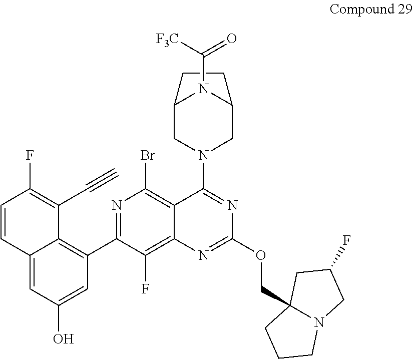 [SMILES: C#Cc1c(F)ccc2cc(O)cc(-c3nc(Br)c4c(N5CC6CCC(C5)N6C(=O)C(F)(F)F)nc(OC[C@]56CCCN5C[C@@H](F)C6)nc4c3F)c12]

1-(3-(5 -bromo-7-(8-ethynyl-7-fluoro-3-hydroxynaphthal en-1-yl)-8-fluoro-2-(((2 S,7aR)-2-fluorotetrahydro-1H-pyrrolizin-7a(5H)-yl)methoxy)pyrido[4,3-d]pyrimidin-4-yl)-3,8-diazabicyclo[3.2.1] octan-8-yl)-2,2,2-trifluoroethan-1-one

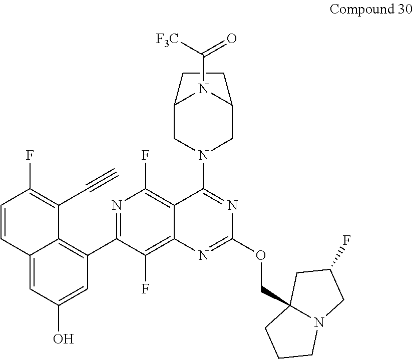 [SMILES: C#Cc1c(F)ccc2cc(O)cc(-c3nc(F)c4c(N5CC6CCC(C5)N6C(=O)C(F)(F)F)nc(OC[C@]56CCCN5C[C@@H](F)C6)nc4c3F)c12]

1-(3-(7-(8-ethynyl-7-fluoro-3-hydroxynaphthalen-l-yl)-5,8-difluoro-2-(((2 S,7aR)-2-fluorotetrahydro-1H-pyrrolizin-7a(5H)-yl)methoxy)pyrido[4,3-d]pyrimidin-4-yl)-3,8-diazabicyclo[3.2.1] octan-8-yl)-2,2,2-trifluoroethan-1-one

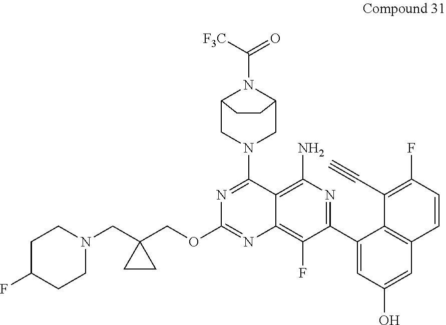 [SMILES: C#Cc1c(F)ccc2cc(O)cc(-c3nc(N)c4c(N5CC6CCC(C5)N6C(=O)C(F)(F)F)nc(OCC5(CN6CCC(F)CC6)CC5)nc4c3F)c12]

1-((1R,5 S)-3-(5-amino-7-(8-ethynyl-7-fluoro-3-hydroxynaphthalen-1-yl)-8-fluoro-2-((1-((4-fluoropiperidin-1-yl)methyl)cyclopropyl)methoxy)pyrido[4,3-d]pyrimidin-4-yl)-3,8-diazabicyclo[3.2.1] octan-8-yl)-2,2,2-trifluoroethan-1-one

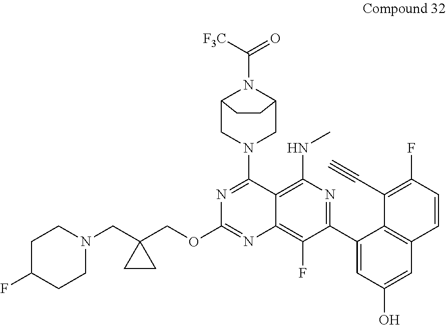 [SMILES: C#Cc1c(F)ccc2cc(O)cc(-c3nc(NC)c4c(N5CC6CCC(C5)N6C(=O)C(F)(F)F)nc(OCC5(CN6CCC(F)CC6)CC5)nc4c3F)c12]

1-((1R,5 S)-3-(7-(8-ethynyl-7-fluoro-3-hydroxynaphthalen-1-yl)-8-fluoro-2-((1-((4-fluoropiperidi n-1-yl)methyl)cyclopropyl)methoxy)-5-(methyl amino)pyrido[4,3-d]pyrimidin-4-yl)-3,8-diazabicyclo[3.2.1] octan-8-yl)-2,2,2-trifluoroethan-1-one

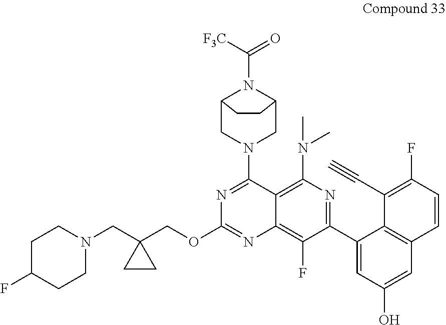 [SMILES: C#Cc1c(F)ccc2cc(O)cc(-c3nc(N(C)C)c4c(N5CC6CCC(C5)N6C(=O)C(F)(F)F)nc(OCC5(CN6CCC(F)CC6)CC5)nc4c3F)c12]

1-((1R,5 S)-3-(5-(dimethyl amino)-7-(8-ethynyl -7-fluoro-3-hydroxynaphthalen-l-yl)-8-fluoro-2-((1-((4-fluoropiperidin-1-yl)methyl)cyclopropyl)methoxy)pyrido[4,3-d]pyrimidin-4-yl)-3,8-diazabicyclo[3.2.1] octan-8-yl)-2,2,2-trifluoroethan-1-one

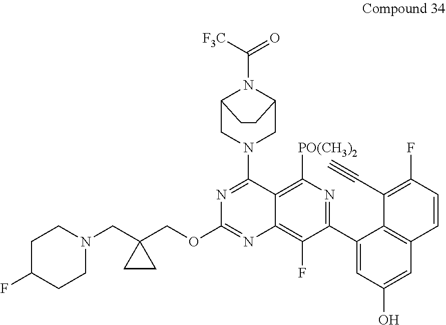

1-((1R,5 S)-3-(5-(dimethylphosphoryl)-7-(8-ethynyl -7-fluoro-3-hydroxynaphthalen-1-yl)-8-fluoro-2-((1-((4-fluoropiperidin-1-yl)methyl)cyclopropyl)methoxy)pyrido[4,3-d]pyrimidin-4-yl)-3,8-diazabicyclo[3.2.1] octan-8-yl)-2,2,2-trifluoroethan-1-one

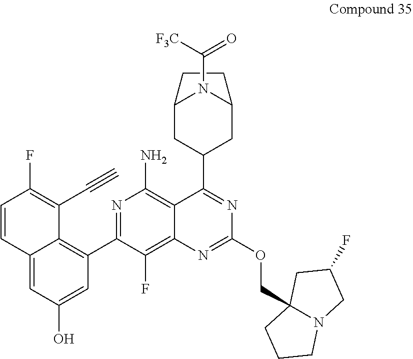 [SMILES: C#Cc1c(F)ccc2cc(O)cc(-c3nc(N)c4c(C5CC6CCC(C5)N6C(=O)C(F)(F)F)nc(OC[C@]56CCCN5C[C@@H](F)C6)nc4c3F)c12]

1-(3-(5 -amino-7-(8-ethynyl-7-fluoro-3-hydroxynaphthalen-l-yl)-8-fluoro-2-(((2 S,7aR)-2-fluorotetrahydro-1H-pyrrolizin-7a(5H)-yl)methoxy)pyrido[4,3-d]pyrimidin-4-yl)-3,8-diazabicyclo[3.2.1] octan-8-yl)-2,2,2-trifluoroethan-1-one

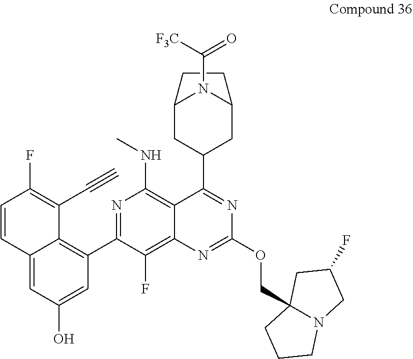 [SMILES: C#Cc1c(F)ccc2cc(O)cc(-c3nc(NC)c4c(C5CC6CCC(C5)N6C(=O)C(F)(F)F)nc(OC[C@]56CCCN5C[C@@H](F)C6)nc4c3F)c12]

1-(3-(7-(8-ethynyl-7-fluoro-3-hydroxynaphthal en-1-yl)-8-fluoro-2-(((2 S,7aR)-2-fluorotetrahydro-1H-pyrrolizin-7a(5H)-yl)methoxy)-5-(methyl amino)pyrido[4,3-d]pyrimidin-4-yl)-3,8-diazabicyclo[3.2.1] octan-8-yl)-2,2,2-trifluoroethan-1-one

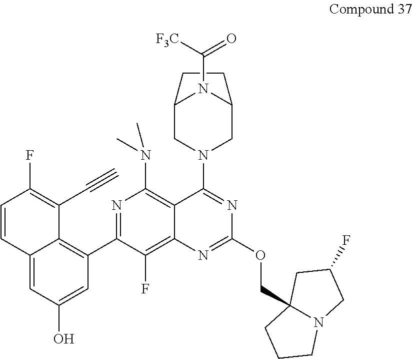 [SMILES: C#Cc1c(F)ccc2cc(O)cc(-c3nc(N(C)C)c4c(N5CC6CCC(C5)N6C(=O)C(F)(F)F)nc(OC[C@]56CCCN5C[C@@H](F)C6)nc4c3F)c12]

1-(3-(5 -(dimethyl amino)-7-(8-ethynyl-7-fluoro-3-hydroxynaphthal en-1-yl)-8-fluoro-2-(((2 S,7aR)-2-fluorotetrahydro-1H-pyrrolizin-7a(5H)-yl)methoxy)pyrido[4,3-d]pyrimidin-4-y1)-3,8-diazabicyclo[3.2.1] octan-8-yl)-2,2,2-trifluoroethan-1-one

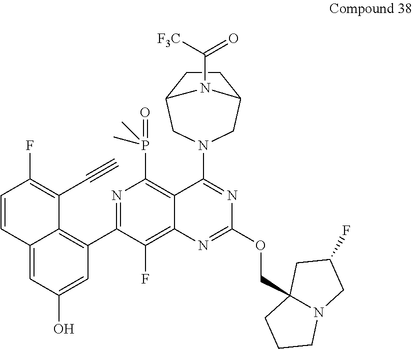 [SMILES: C#Cc1c(F)ccc2cc(O)cc(-c3nc(P(C)(C)=O)c4c(N5CC6CCC(C5)N6C(=O)C(F)(F)F)nc(OC[C@]56CCCN5C[C@@H](F)C6)nc4c3F)c12]

1-(3-(5 -(dimethylphosphoryl)-7-(8-ethynyl-7-fluoro-3-hydroxynaphthal en-1-yl)-8-fluoro-2-(((2 S,7aR)-2-fluorotetrahydro-1H-pyrrolizin-7a(5H)-yl)methoxy)pyrido[4,3-d]pyrimidin-4-y1)-3,8-diazabicyclo[3.2.1] octan-8-yl)-2,2,2-trifluoroethan-1-one

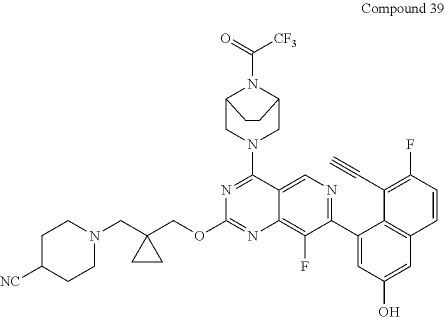 [SMILES: C#Cc1c(F)ccc2cc(O)cc(-c3ncc4c(N5CC6CCC(C5)N6C(=O)C(F)(F)F)nc(OCC5(CN6CCC(C#N)CC6)CC5)nc4c3F)c12]

1-((14(7-(8-ethynyl-7-fluoro-3-hydroxynaphthalen-l-yl)-8-fluoro-441R,5 S)-8-(2,2,2-trifluoroacetyl)-3,8-diazabicyclo[3.2.1] octan-3-yl)pyrido[4,3-d]pyrimidin-2-yl)oxy)methyl)cyclopropyl)methyl)piperidine-4-carbonitrile

Additional compounds which are contemplated and part of the present disclosure include the following.

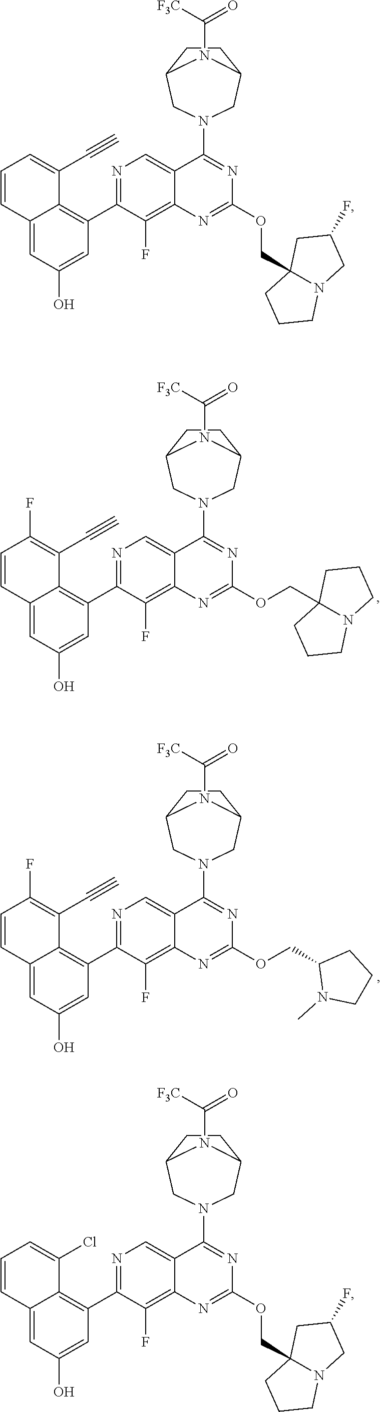 [SMILES: C#Cc1cccc2cc(O)cc(-c3ncc4c(N5CC6CCC(C5)N6C(=O)C(F)(F)F)nc(OC[C@]56CCCN5C[C@@H](F)C6)nc4c3F)c12 ; C#Cc1c(F)ccc2cc(O)cc(-c3ncc4c(N5CC6CCC(C5)N6C(=O)C(F)(F)F)nc(OCC56CCCN5CCC6)nc4c3F)c12 ; C#Cc1c(F)ccc2cc(O)cc(-c3ncc4c(N5CC6CCC(C5)N6C(=O)C(F)(F)F)nc(OC[C@@H]5CCCN5C)nc4c3F)c12 ; O=C(N1C2CCC1CN(c1nc(OC[C@]34CCCN3C[C@@H](F)C4)nc3c(F)c(-c4cc(O)cc5cccc(Cl)c45)ncc13)C2)C(F)(F)F]

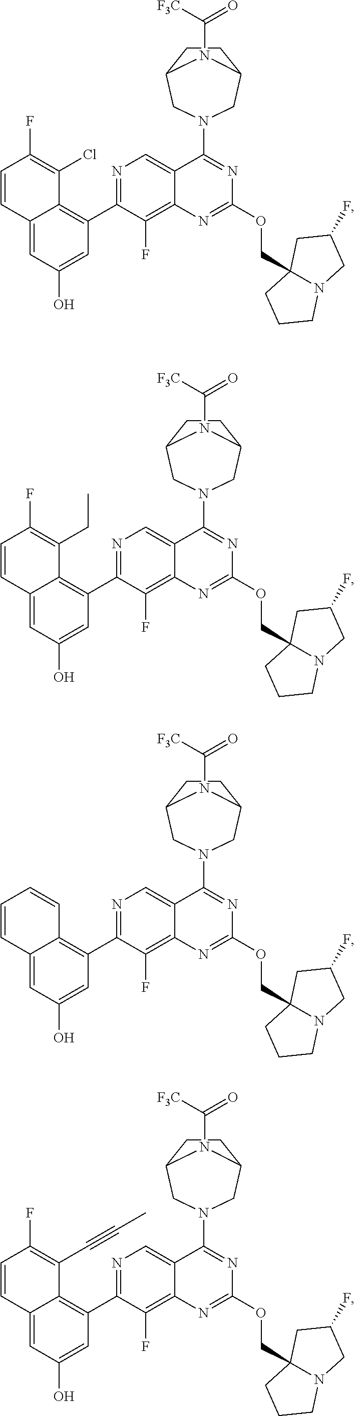 [SMILES: O=C(N1C2CCC1CN(c1nc(OC[C@]34CCCN3C[C@@H](F)C4)nc3c(F)c(-c4cc(O)cc5ccc(F)c(Cl)c45)ncc13)C2)C(F)(F)F ; CCc1c(F)ccc2cc(O)cc(-c3ncc4c(N5CC6CCC(C5)N6C(=O)C(F)(F)F)nc(OC[C@]56CCCN5C[C@@H](F)C6)nc4c3F)c12 ; O=C(N1C2CCC1CN(c1nc(OC[C@]34CCCN3C[C@@H](F)C4)nc3c(F)c(-c4cc(O)cc5ccccc45)ncc13)C2)C(F)(F)F ; CC#Cc1c(F)ccc2cc(O)cc(-c3ncc4c(N5CC6CCC(C5)N6C(=O)C(F)(F)F)nc(OC[C@]56CCCN5C[C@@H](F)C6)nc4c3F)c12]

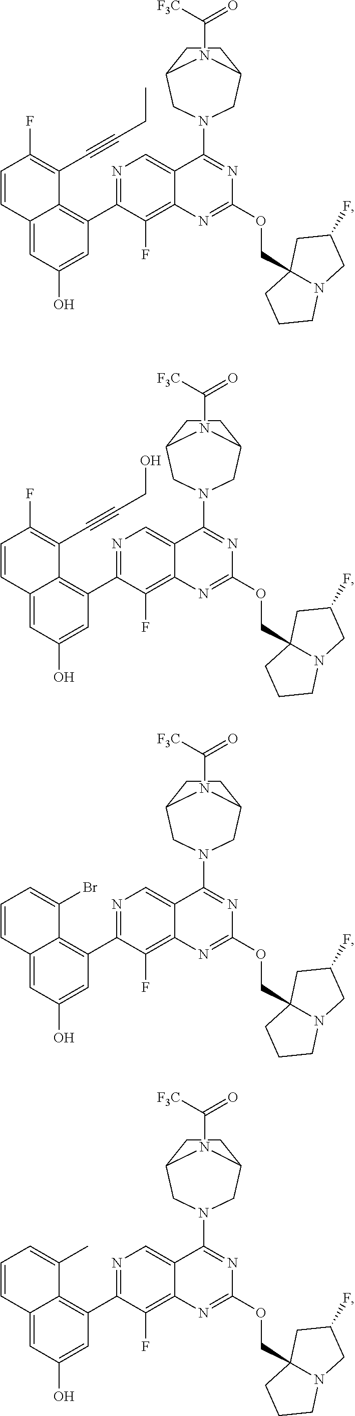 [SMILES: CCC#Cc1c(F)ccc2cc(O)cc(-c3ncc4c(N5CC6CCC(C5)N6C(=O)C(F)(F)F)nc(OC[C@]56CCCN5C[C@@H](F)C6)nc4c3F)c12 ; O=C(N1C2CCC1CN(c1nc(OC[C@]34CCCN3C[C@@H](F)C4)nc3c(F)c(-c4cc(O)cc5ccc(F)c(C#CCO)c45)ncc13)C2)C(F)(F)F ; O=C(N1C2CCC1CN(c1nc(OC[C@]34CCCN3C[C@@H](F)C4)nc3c(F)c(-c4cc(O)cc5cccc(Br)c45)ncc13)C2)C(F)(F)F ; Cc1cccc2cc(O)cc(-c3ncc4c(N5CC6CCC(C5)N6C(=O)C(F)(F)F)nc(OC[C@]56CCCN5C[C@@H](F)C6)nc4c3F)c12]

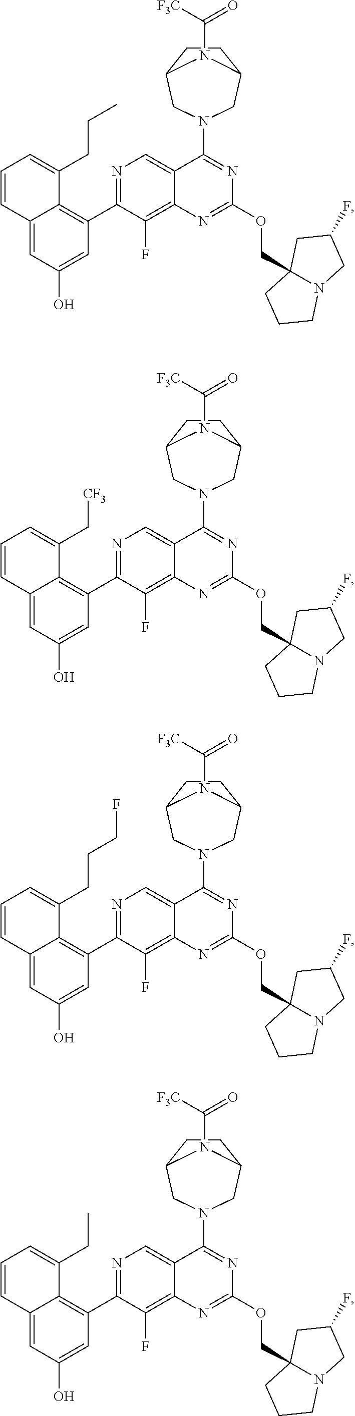 [SMILES: CCCc1cccc2cc(O)cc(-c3ncc4c(N5CC6CCC(C5)N6C(=O)C(F)(F)F)nc(OC[C@]56CCCN5C[C@@H](F)C6)nc4c3F)c12 ; O=C(N1C2CCC1CN(c1nc(OC[C@]34CCCN3C[C@@H](F)C4)nc3c(F)c(-c4cc(O)cc5cccc(CC(F)(F)F)c45)ncc13)C2)C(F)(F)F ; O=C(N1C2CCC1CN(c1nc(OC[C@]34CCCN3C[C@@H](F)C4)nc3c(F)c(-c4cc(O)cc5cccc(CCCF)c45)ncc13)C2)C(F)(F)F ; CCc1cccc2cc(O)cc(-c3ncc4c(N5CC6CCC(C5)N6C(=O)C(F)(F)F)nc(OC[C@]56CCCN5C[C@@H](F)C6)nc4c3F)c12]

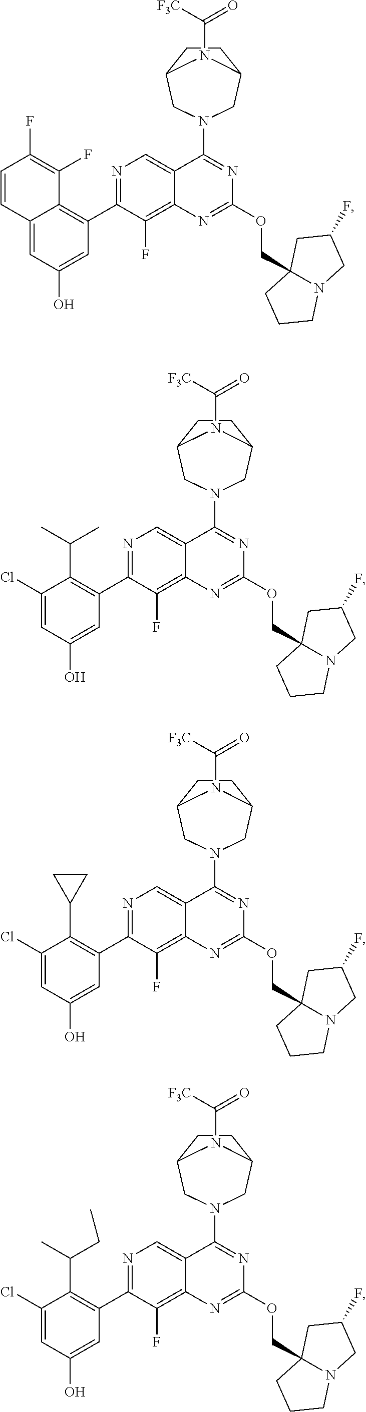 [SMILES: O=C(N1C2CCC1CN(c1nc(OC[C@]34CCCN3C[C@@H](F)C4)nc3c(F)c(-c4cc(O)cc5ccc(F)c(F)c45)ncc13)C2)C(F)(F)F ; CC(C)c1c(Cl)cc(O)cc1-c1ncc2c(N3CC4CCC(C3)N4C(=O)C(F)(F)F)nc(OC[C@]34CCCN3C[C@@H](F)C4)nc2c1F ; O=C(N1C2CCC1CN(c1nc(OC[C@]34CCCN3C[C@@H](F)C4)nc3c(F)c(-c4cc(O)cc(Cl)c4C4CC4)ncc13)C2)C(F)(F)F ; CCC(C)c1c(Cl)cc(O)cc1-c1ncc2c(N3CC4CCC(C3)N4C(=O)C(F)(F)F)nc(OC[C@]34CCCN3C[C@@H](F)C4)nc2c1F]

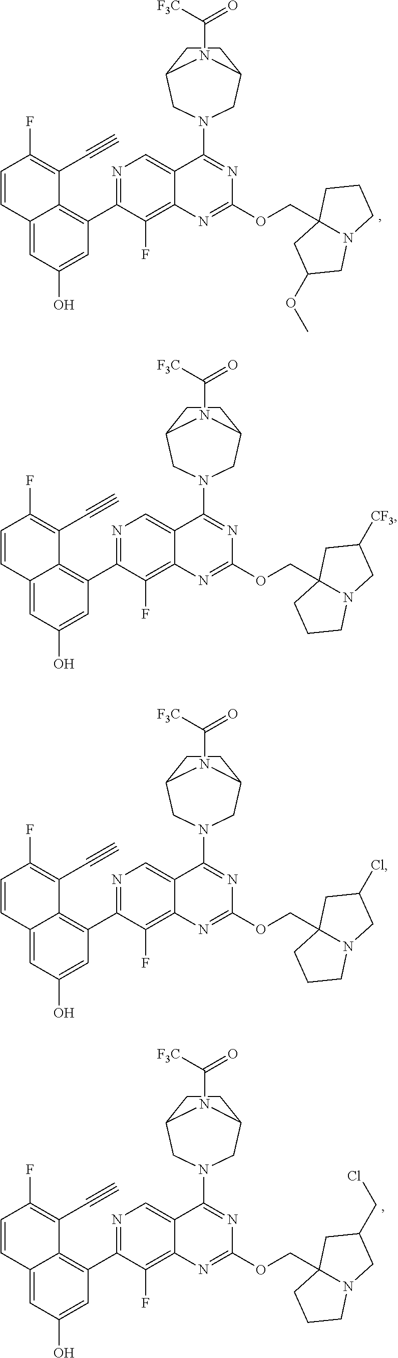 [SMILES: C#Cc1c(F)ccc2cc(O)cc(-c3ncc4c(N5CC6CCC(C5)N6C(=O)C(F)(F)F)nc(OCC56CCCN5CC(OC)C6)nc4c3F)c12 ; C#Cc1c(F)ccc2cc(O)cc(-c3ncc4c(N5CC6CCC(C5)N6C(=O)C(F)(F)F)nc(OCC56CCCN5CC(C(F)(F)F)C6)nc4c3F)c12 ; C#Cc1c(F)ccc2cc(O)cc(-c3ncc4c(N5CC6CCC(C5)N6C(=O)C(F)(F)F)nc(OCC56CCCN5CC(Cl)C6)nc4c3F)c12 ; C#Cc1c(F)ccc2cc(O)cc(-c3ncc4c(N5CC6CCC(C5)N6C(=O)C(F)(F)F)nc(OCC56CCCN5CC(CCl)C6)nc4c3F)c12]

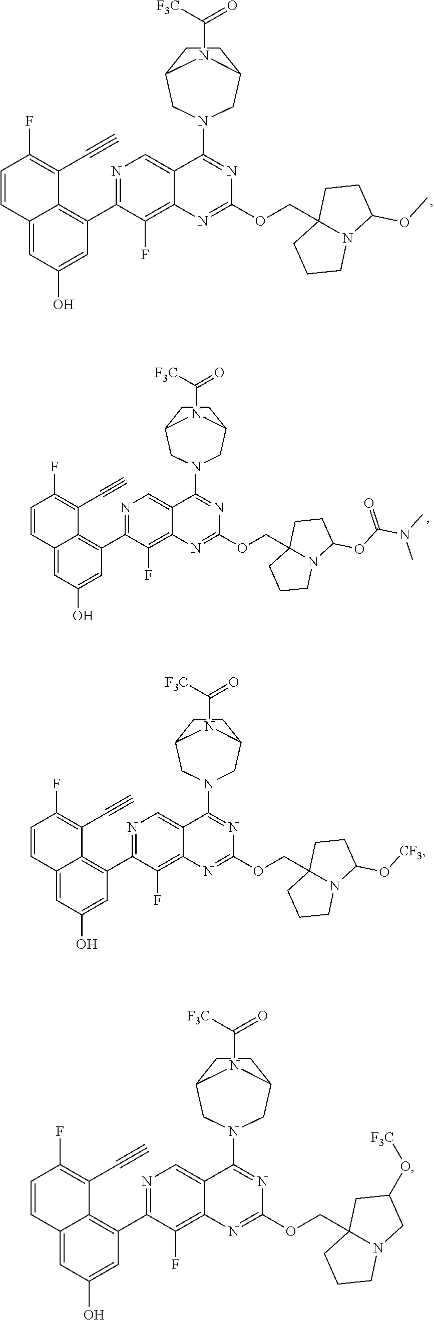 [SMILES: C#Cc1c(F)ccc2cc(O)cc(-c3ncc4c(N5CC6CCC(C5)N6C(=O)C(F)(F)F)nc(OCC56CCCN5C(OC)CC6)nc4c3F)c12 ; C#Cc1c(F)ccc2cc(O)cc(-c3ncc4c(N5CC6CCC(C5)N6C(=O)C(F)(F)F)nc(OCC56CCCN5C(OC(=O)N(C)C)CC6)nc4c3F)c12 ; C#Cc1c(F)ccc2cc(O)cc(-c3ncc4c(N5CC6CCC(C5)N6C(=O)C(F)(F)F)nc(OCC56CCCN5C(OC(F)(F)F)CC6)nc4c3F)c12 ; C#Cc1c(F)ccc2cc(O)cc(-c3ncc4c(N5CC6CCC(C5)N6C(=O)C(F)(F)F)nc(OCC56CCCN5CC(OC(F)(F)F)C6)nc4c3F)c12]

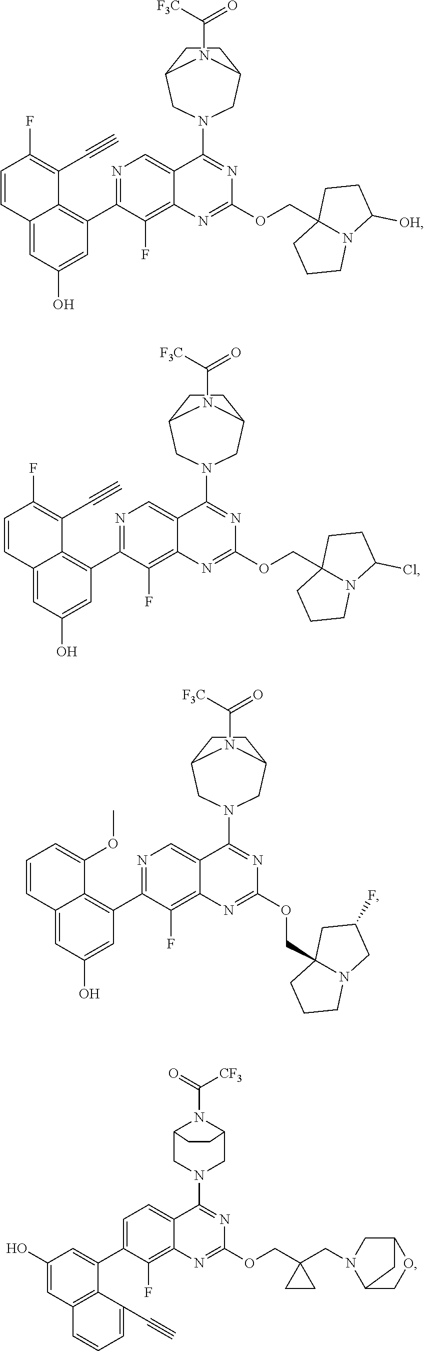 [SMILES: C#Cc1c(F)ccc2cc(O)cc(-c3ncc4c(N5CC6CCC(C5)N6C(=O)C(F)(F)F)nc(OCC56CCCN5C(O)CC6)nc4c3F)c12 ; C#Cc1c(F)ccc2cc(O)cc(-c3ncc4c(N5CC6CCC(C5)N6C(=O)C(F)(F)F)nc(OCC56CCCN5C(Cl)CC6)nc4c3F)c12 ; COc1cccc2cc(O)cc(-c3ncc4c(N5CC6CCC(C5)N6C(=O)C(F)(F)F)nc(OC[C@]56CCCN5C[C@@H](F)C6)nc4c3F)c12 ; C#Cc1cccc2cc(O)cc(-c3ccc4c(N5CC6CCC(C5)N6C(=O)C(F)(F)F)nc(OCC5(CN6CC7CC6CO7)CC5)nc4c3F)c12]

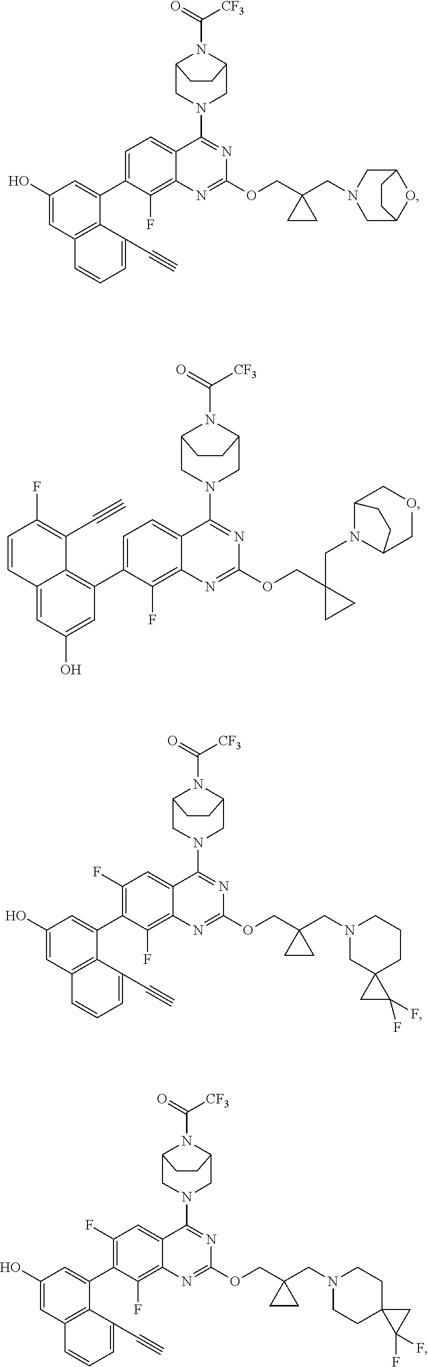 [SMILES: C#Cc1cccc2cc(O)cc(-c3ccc4c(N5CC6CCC(C5)N6C(=O)C(F)(F)F)nc(OCC5(CN6CC7CCC(C6)O7)CC5)nc4c3F)c12 ; C#Cc1c(F)ccc2cc(O)cc(-c3ccc4c(N5CC6CCC(C5)N6C(=O)C(F)(F)F)nc(OCC5(CN6C7CCC6COC7)CC5)nc4c3F)c12 ; C#Cc1cccc2cc(O)cc(-c3c(F)cc4c(N5CC6CCC(C5)N6C(=O)C(F)(F)F)nc(OCC5(CN6CCCC7(C6)CC7(F)F)CC5)nc4c3F)c12 ; C#Cc1cccc2cc(O)cc(-c3c(F)cc4c(N5CC6CCC(C5)N6C(=O)C(F)(F)F)nc(OCC5(CN6CCC7(CC6)CC7(F)F)CC5)nc4c3F)c12]

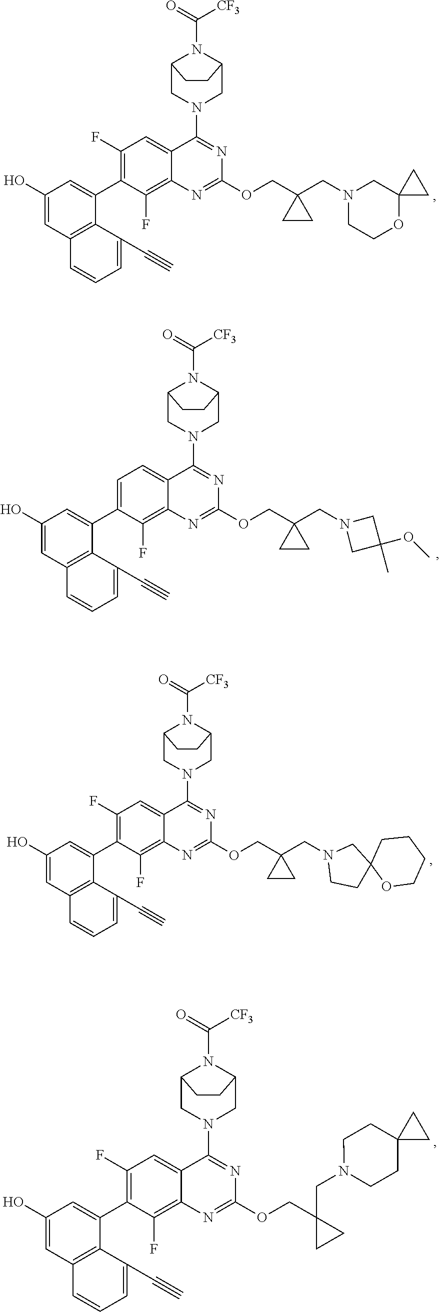 [SMILES: C#Cc1cccc2cc(O)cc(-c3c(F)cc4c(N5CC6CCC(C5)N6C(=O)C(F)(F)F)nc(OCC5(CN6CCOC7(CC7)C6)CC5)nc4c3F)c12 ; C#Cc1cccc2cc(O)cc(-c3ccc4c(N5CC6CCC(C5)N6C(=O)C(F)(F)F)nc(OCC5(CN6CC(C)(OC)C6)CC5)nc4c3F)c12 ; C#Cc1cccc2cc(O)cc(-c3c(F)cc4c(N5CC6CCC(C5)N6C(=O)C(F)(F)F)nc(OCC5(CN6CCC7(CCCCO7)C6)CC5)nc4c3F)c12 ; C#Cc1cccc2cc(O)cc(-c3c(F)cc4c(N5CC6CCC(C5)N6C(=O)C(F)(F)F)nc(OCC5(CN6CCC7(CC6)CC7)CC5)nc4c3F)c12]

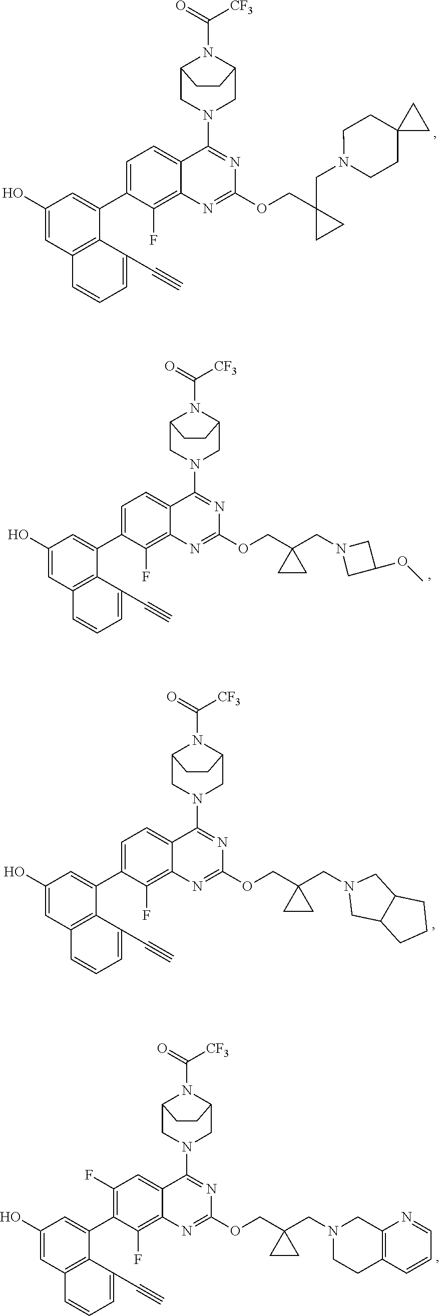 [SMILES: C#Cc1cccc2cc(O)cc(-c3ccc4c(N5CC6CCC(C5)N6C(=O)C(F)(F)F)nc(OCC5(CN6CCC7(CC6)CC7)CC5)nc4c3F)c12 ; C#Cc1cccc2cc(O)cc(-c3ccc4c(N5CC6CCC(C5)N6C(=O)C(F)(F)F)nc(OCC5(CN6CC(OC)C6)CC5)nc4c3F)c12 ; C#Cc1cccc2cc(O)cc(-c3ccc4c(N5CC6CCC(C5)N6C(=O)C(F)(F)F)nc(OCC5(CN6CC7CCCC7C6)CC5)nc4c3F)c12 ; C#Cc1cccc2cc(O)cc(-c3c(F)cc4c(N5CC6CCC(C5)N6C(=O)C(F)(F)F)nc(OCC5(CN6CCc7cccnc7C6)CC5)nc4c3F)c12]

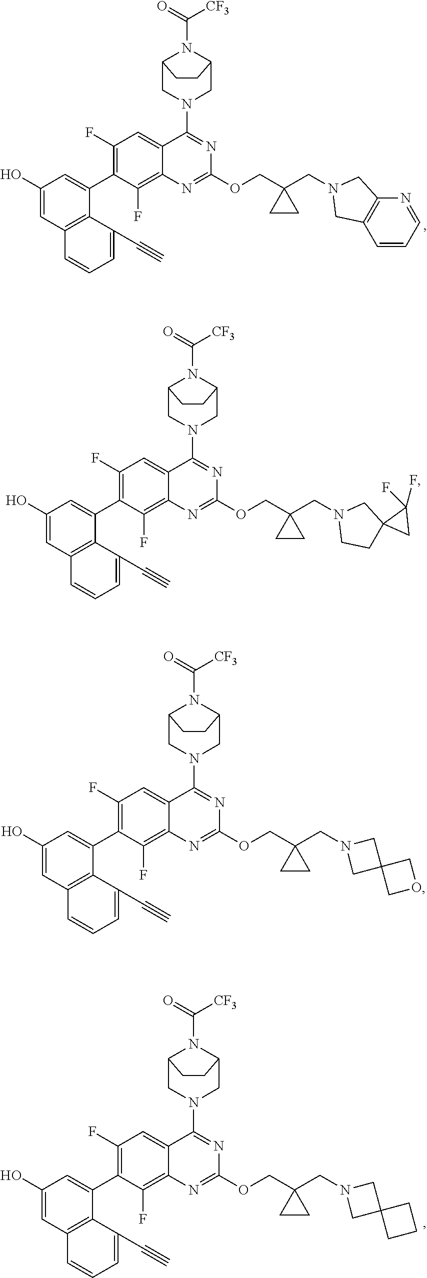 [SMILES: C#Cc1cccc2cc(O)cc(-c3c(F)cc4c(N5CC6CCC(C5)N6C(=O)C(F)(F)F)nc(OCC5(CN6Cc7cccnc7C6)CC5)nc4c3F)c12 ; C#Cc1cccc2cc(O)cc(-c3c(F)cc4c(N5CC6CCC(C5)N6C(=O)C(F)(F)F)nc(OCC5(CN6CCC7(C6)CC7(F)F)CC5)nc4c3F)c12 ; C#Cc1cccc2cc(O)cc(-c3c(F)cc4c(N5CC6CCC(C5)N6C(=O)C(F)(F)F)nc(OCC5(CN6CC7(COC7)C6)CC5)nc4c3F)c12 ; C#Cc1cccc2cc(O)cc(-c3c(F)cc4c(N5CC6CCC(C5)N6C(=O)C(F)(F)F)nc(OCC5(CN6CC7(CCC7)C6)CC5)nc4c3F)c12]

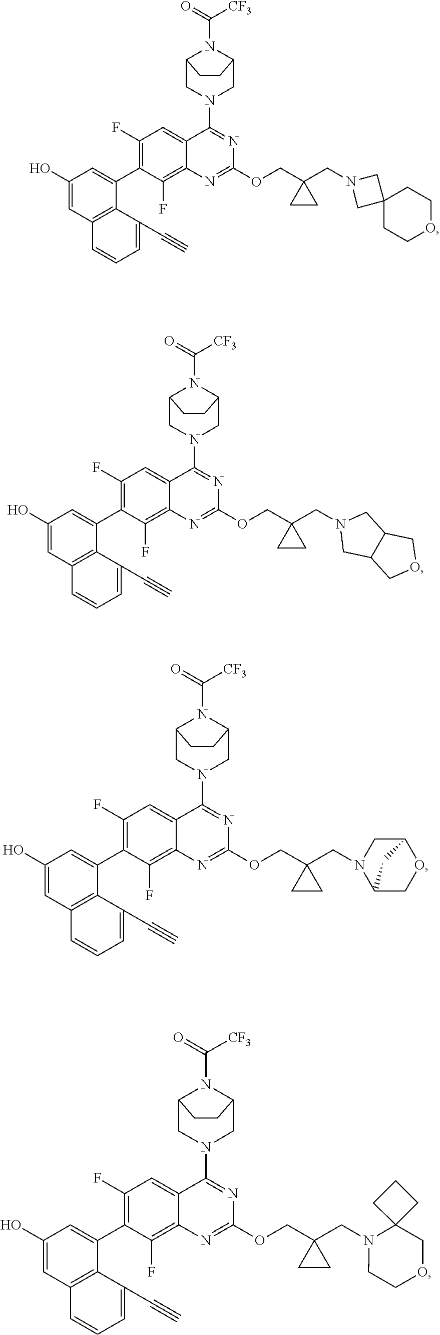

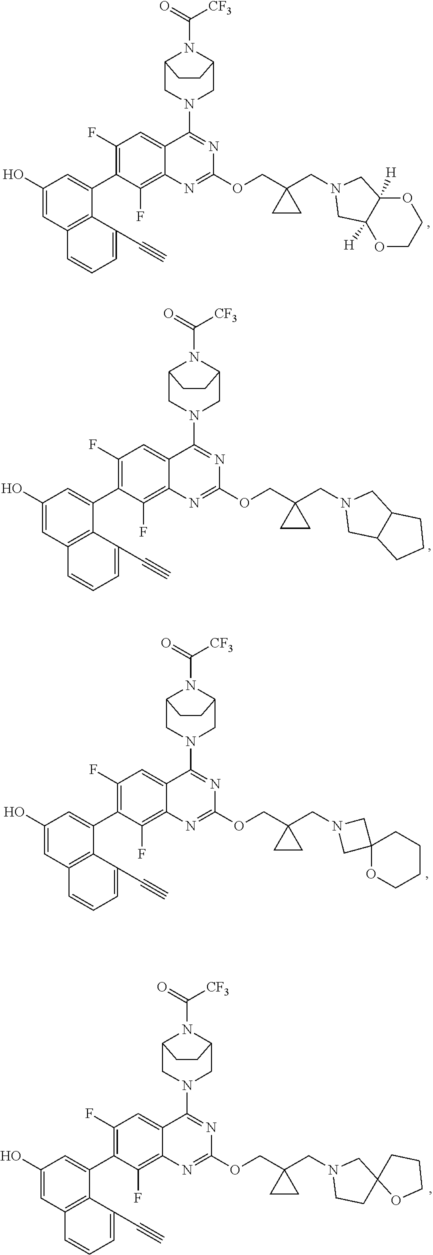 [SMILES: C#Cc1cccc2cc(O)cc(-c3c(F)cc4c(N5CC6CCC(C5)N6C(=O)C(F)(F)F)nc(OCC5(CN6C[C@@H]7OCCO[C@@H]7C6)CC5)nc4c3F)c12 ; C#Cc1cccc2cc(O)cc(-c3c(F)cc4c(N5CC6CCC(C5)N6C(=O)C(F)(F)F)nc(OCC5(CN6CC7CCCC7C6)CC5)nc4c3F)c12 ; C#Cc1cccc2cc(O)cc(-c3c(F)cc4c(N5CC6CCC(C5)N6C(=O)C(F)(F)F)nc(OCC5(CN6CC7(CCCCO7)C6)CC5)nc4c3F)c12 ; C#Cc1cccc2cc(O)cc(-c3c(F)cc4c(N5CC6CCC(C5)N6C(=O)C(F)(F)F)nc(OCC5(CN6CCC7(CCCO7)C6)CC5)nc4c3F)c12]

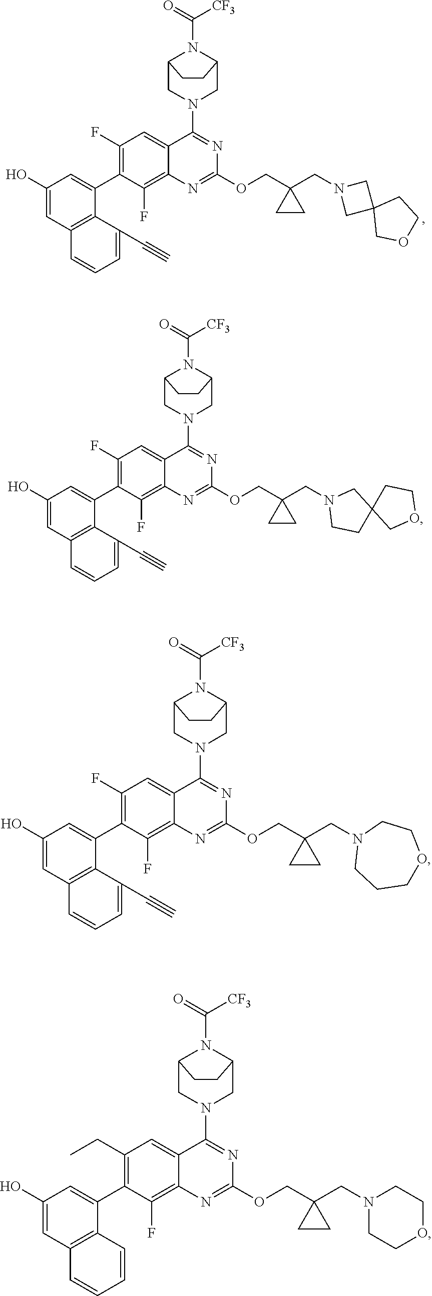 [SMILES: C#Cc1cccc2cc(O)cc(-c3c(F)cc4c(N5CC6CCC(C5)N6C(=O)C(F)(F)F)nc(OCC5(CN6CC7(CCOC7)C6)CC5)nc4c3F)c12 ; C#Cc1cccc2cc(O)cc(-c3c(F)cc4c(N5CC6CCC(C5)N6C(=O)C(F)(F)F)nc(OCC5(CN6CCC7(CCOC7)C6)CC5)nc4c3F)c12 ; C#Cc1cccc2cc(O)cc(-c3c(F)cc4c(N5CC6CCC(C5)N6C(=O)C(F)(F)F)nc(OCC5(CN6CCCOCC6)CC5)nc4c3F)c12 ; CCc1cc2c(N3CC4CCC(C3)N4C(=O)C(F)(F)F)nc(OCC3(CN4CCOCC4)CC3)nc2c(F)c1-c1cc(O)cc2ccccc12]

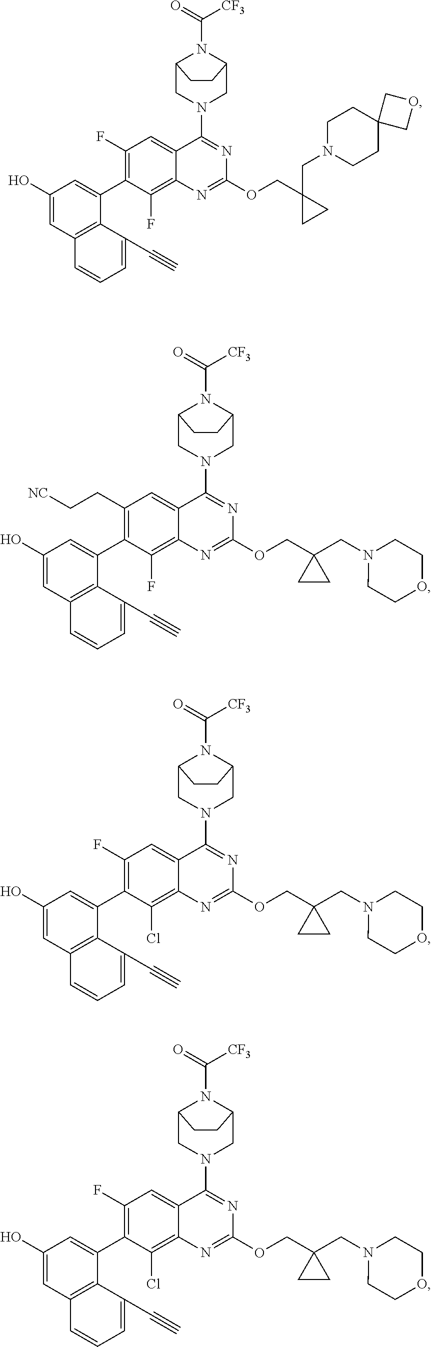 [SMILES: C#Cc1cccc2cc(O)cc(-c3c(F)cc4c(N5CC6CCC(C5)N6C(=O)C(F)(F)F)nc(OCC5(CN6CCC7(CC6)COC7)CC5)nc4c3F)c12 ; C#Cc1cccc2cc(O)cc(-c3c(CCC#N)cc4c(N5CC6CCC(C5)N6C(=O)C(F)(F)F)nc(OCC5(CN6CCOCC6)CC5)nc4c3F)c12 ; C#Cc1cccc2cc(O)cc(-c3c(F)cc4c(N5CC6CCC(C5)N6C(=O)C(F)(F)F)nc(OCC5(CN6CCOCC6)CC5)nc4c3Cl)c12 ; C#Cc1cccc2cc(O)cc(-c3c(F)cc4c(N5CC6CCC(C5)N6C(=O)C(F)(F)F)nc(OCC5(CN6CCOCC6)CC5)nc4c3Cl)c12]

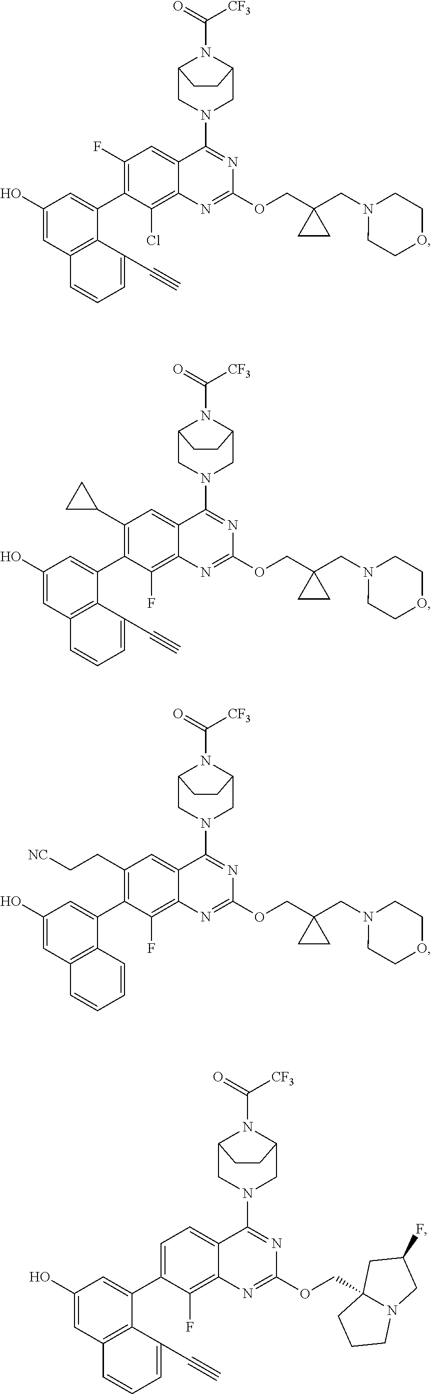 [SMILES: C#Cc1cccc2cc(O)cc(-c3c(F)cc4c(N5CC6CCC(C5)N6C(=O)C(F)(F)F)nc(OCC5(CN6CCOCC6)CC5)nc4c3Cl)c12 ; C#Cc1cccc2cc(O)cc(-c3c(C4CC4)cc4c(N5CC6CCC(C5)N6C(=O)C(F)(F)F)nc(OCC5(CN6CCOCC6)CC5)nc4c3F)c12 ; N#CCCc1cc2c(N3CC4CCC(C3)N4C(=O)C(F)(F)F)nc(OCC3(CN4CCOCC4)CC3)nc2c(F)c1-c1cc(O)cc2ccccc12 ; C#Cc1cccc2cc(O)cc(-c3ccc4c(N5CC6CCC(C5)N6C(=O)C(F)(F)F)nc(OC[C@@]56CCCN5C[C@H](F)C6)nc4c3F)c12]

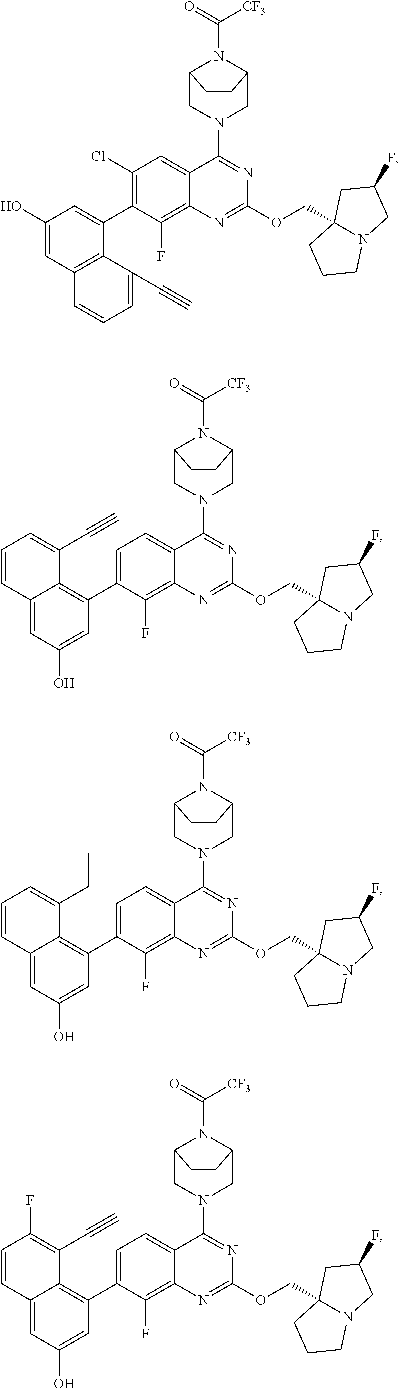 [SMILES: C#Cc1cccc2cc(O)cc(-c3c(Cl)cc4c(N5CC6CCC(C5)N6C(=O)C(F)(F)F)nc(OC[C@@]56CCCN5C[C@H](F)C6)nc4c3F)c12 ; C#Cc1cccc2cc(O)cc(-c3ccc4c(N5CC6CCC(C5)N6C(=O)C(F)(F)F)nc(OC[C@@]56CCCN5C[C@H](F)C6)nc4c3F)c12 ; CCc1cccc2cc(O)cc(-c3ccc4c(N5CC6CCC(C5)N6C(=O)C(F)(F)F)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)C(F)(F)F)nc(OC[C@@]56CCCN5C[C@H](F)C6)nc4c3F)c12]

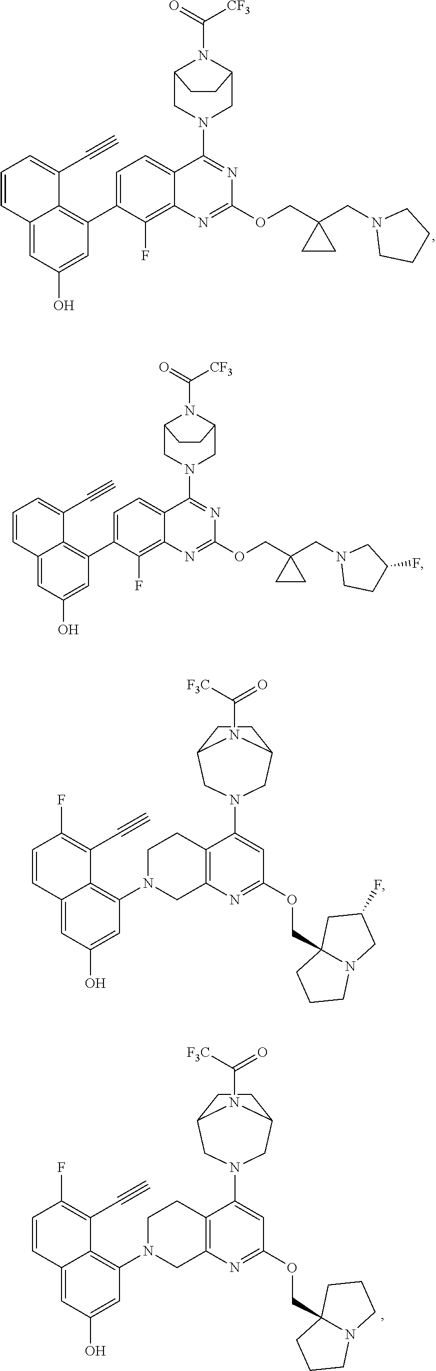

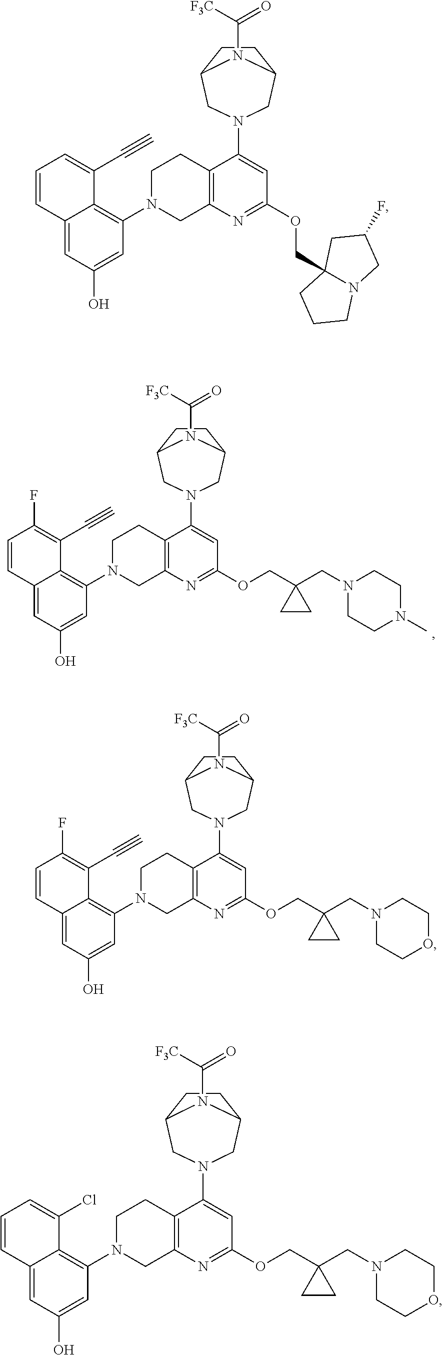 [SMILES: C#Cc1cccc2cc(O)cc(N3CCc4c(N5CC6CCC(C5)N6C(=O)C(F)(F)F)cc(OC[C@]56CCCN5C[C@@H](F)C6)nc4C3)c12 ; C#Cc1c(F)ccc2cc(O)cc(N3CCc4c(N5CC6CCC(C5)N6C(=O)C(F)(F)F)cc(OCC5(CN6CCN(C)CC6)CC5)nc4C3)c12 ; C#Cc1c(F)ccc2cc(O)cc(N3CCc4c(N5CC6CCC(C5)N6C(=O)C(F)(F)F)cc(OCC5(CN6CCOCC6)CC5)nc4C3)c12 ; O=C(N1C2CCC1CN(c1cc(OCC3(CN4CCOCC4)CC3)nc3c1CCN(c1cc(O)cc4cccc(Cl)c14)C3)C2)C(F)(F)F]

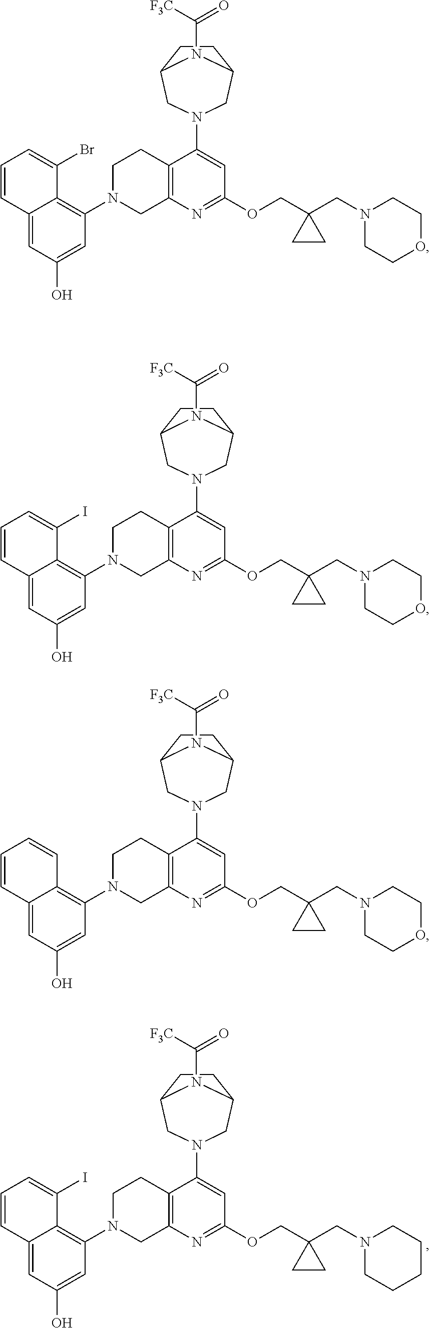 [SMILES: O=C(N1C2CCC1CN(c1cc(OCC3(CN4CCOCC4)CC3)nc3c1CCN(c1cc(O)cc4cccc(Br)c14)C3)C2)C(F)(F)F ; O=C(N1C2CCC1CN(c1cc(OCC3(CN4CCOCC4)CC3)nc3c1CCN(c1cc(O)cc4cccc(I)c14)C3)C2)C(F)(F)F ; O=C(N1C2CCC1CN(c1cc(OCC3(CN4CCOCC4)CC3)nc3c1CCN(c1cc(O)cc4ccccc14)C3)C2)C(F)(F)F ; O=C(N1C2CCC1CN(c1cc(OCC3(CN4CCCCC4)CC3)nc3c1CCN(c1cc(O)cc4cccc(I)c14)C3)C2)C(F)(F)F]

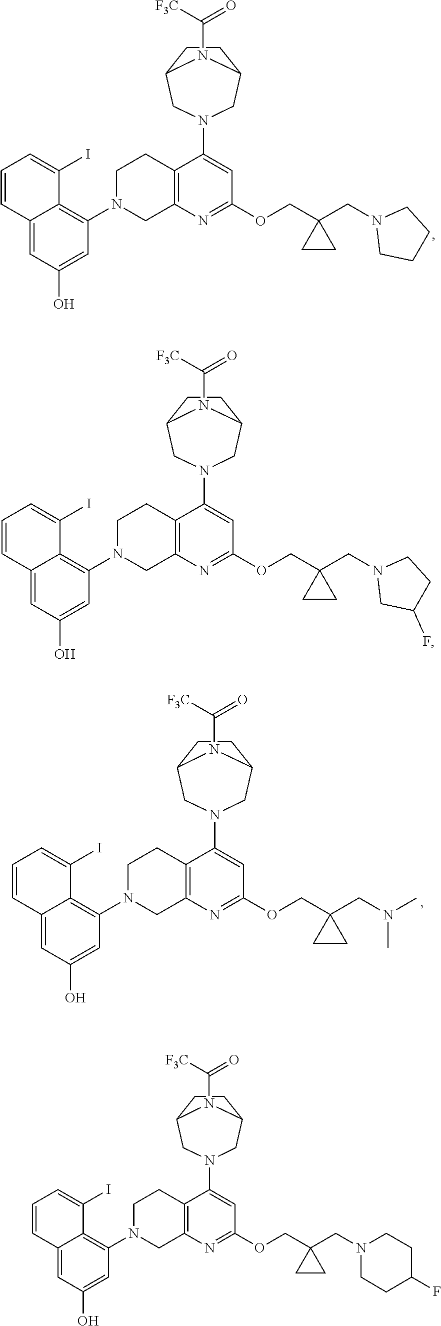 [SMILES: O=C(N1C2CCC1CN(c1cc(OCC3(CN4CCCC4)CC3)nc3c1CCN(c1cc(O)cc4cccc(I)c14)C3)C2)C(F)(F)F ; O=C(N1C2CCC1CN(c1cc(OCC3(CN4CCC(F)C4)CC3)nc3c1CCN(c1cc(O)cc4cccc(I)c14)C3)C2)C(F)(F)F ; CN(C)CC1(COc2cc(N3CC4CCC(C3)N4C(=O)C(F)(F)F)c3c(n2)CN(c2cc(O)cc4cccc(I)c24)CC3)CC1 ; O=C(N1C2CCC1CN(c1cc(OCC3(CN4CCC(F)CC4)CC3)nc3c1CCN(c1cc(O)cc4cccc(I)c14)C3)C2)C(F)(F)F]

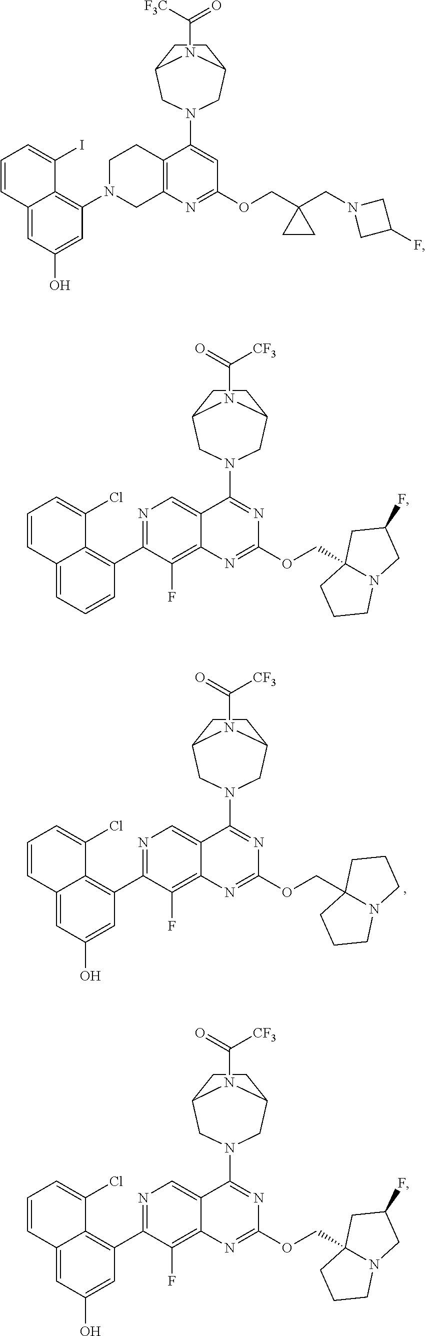 [SMILES: O=C(N1C2CCC1CN(c1cc(OCC3(CN4CC(F)C4)CC3)nc3c1CCN(c1cc(O)cc4cccc(I)c14)C3)C2)C(F)(F)F ; O=C(N1C2CCC1CN(c1nc(OC[C@@]34CCCN3C[C@H](F)C4)nc3c(F)c(-c4cccc5cccc(Cl)c45)ncc13)C2)C(F)(F)F ; O=C(N1C2CCC1CN(c1nc(OCC34CCCN3CCC4)nc3c(F)c(-c4cc(O)cc5cccc(Cl)c45)ncc13)C2)C(F)(F)F ; O=C(N1C2CCC1CN(c1nc(OC[C@@]34CCCN3C[C@H](F)C4)nc3c(F)c(-c4cc(O)cc5cccc(Cl)c45)ncc13)C2)C(F)(F)F]

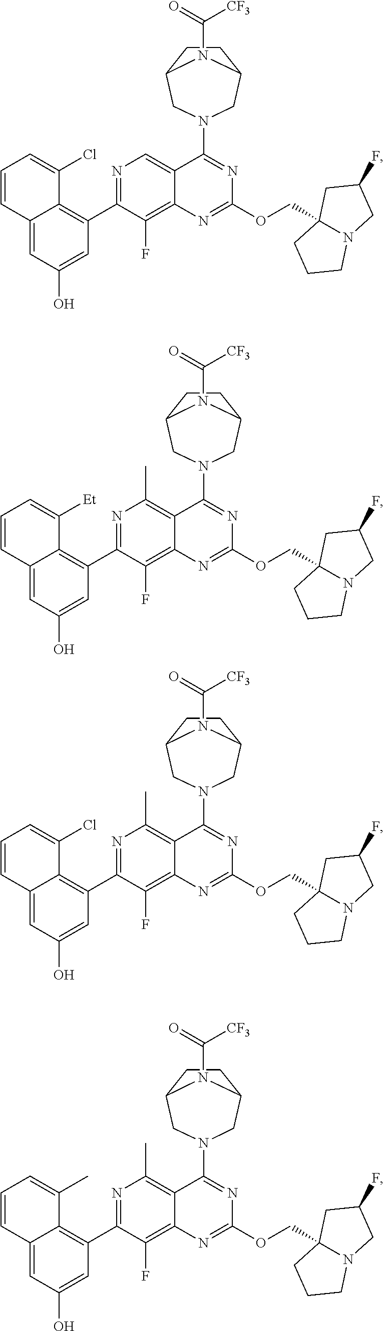 [SMILES: O=C(N1C2CCC1CN(c1nc(OC[C@@]34CCCN3C[C@H](F)C4)nc3c(F)c(-c4cc(O)cc5cccc(Cl)c45)ncc13)C2)C(F)(F)F ; CCc1cccc2cc(O)cc(-c3nc(C)c4c(N5CC6CCC(C5)N6C(=O)C(F)(F)F)nc(OC[C@@]56CCCN5C[C@H](F)C6)nc4c3F)c12 ; Cc1nc(-c2cc(O)cc3cccc(Cl)c23)c(F)c2nc(OC[C@@]34CCCN3C[C@H](F)C4)nc(N3CC4CCC(C3)N4C(=O)C(F)(F)F)c12 ; Cc1cccc2cc(O)cc(-c3nc(C)c4c(N5CC6CCC(C5)N6C(=O)C(F)(F)F)nc(OC[C@@]56CCCN5C[C@H](F)C6)nc4c3F)c12]

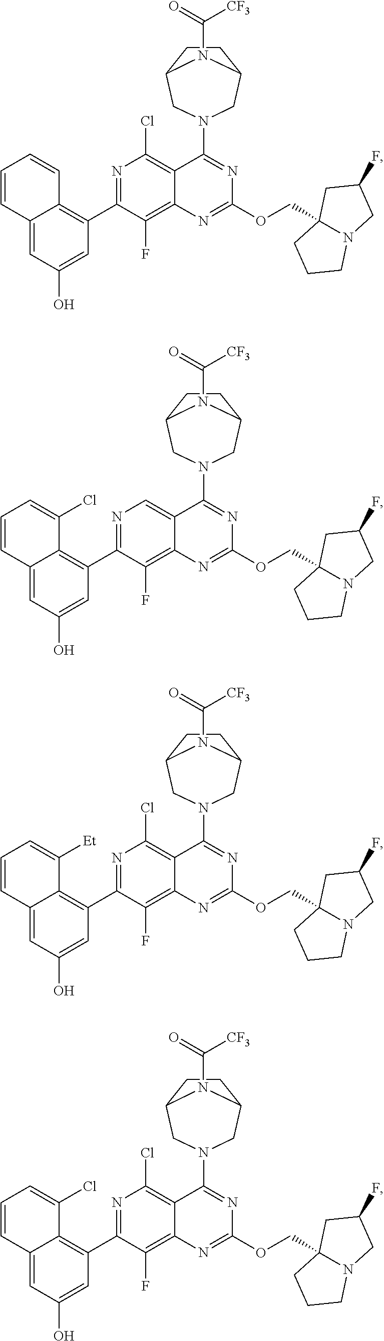 [SMILES: O=C(N1C2CCC1CN(c1nc(OC[C@@]34CCCN3C[C@H](F)C4)nc3c(F)c(-c4cc(O)cc5ccccc45)nc(Cl)c13)C2)C(F)(F)F ; O=C(N1C2CCC1CN(c1nc(OC[C@@]34CCCN3C[C@H](F)C4)nc3c(F)c(-c4cc(O)cc5cccc(Cl)c45)ncc13)C2)C(F)(F)F ; CCc1cccc2cc(O)cc(-c3nc(Cl)c4c(N5CC6CCC(C5)N6C(=O)C(F)(F)F)nc(OC[C@@]56CCCN5C[C@H](F)C6)nc4c3F)c12 ; O=C(N1C2CCC1CN(c1nc(OC[C@@]34CCCN3C[C@H](F)C4)nc3c(F)c(-c4cc(O)cc5cccc(Cl)c45)nc(Cl)c13)C2)C(F)(F)F]

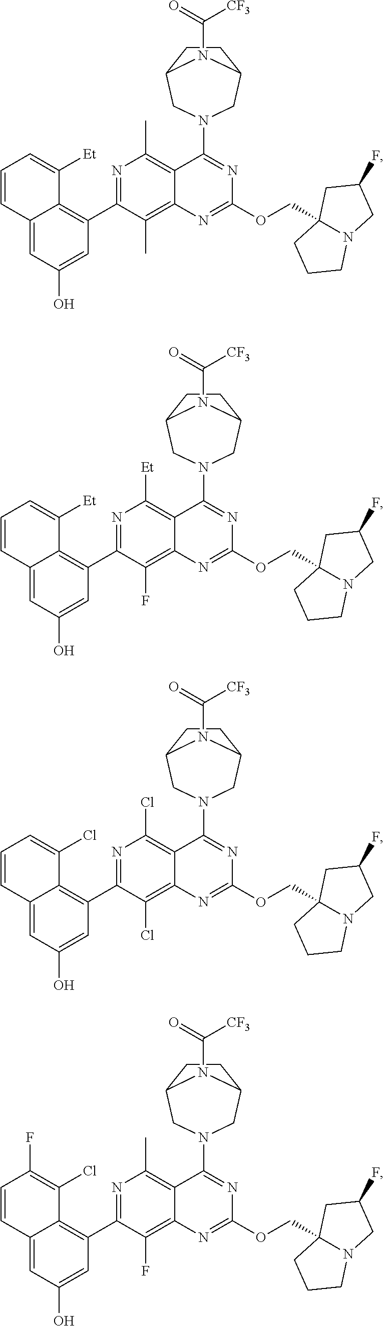 [SMILES: CCc1cccc2cc(O)cc(-c3nc(C)c4c(N5CC6CCC(C5)N6C(=O)C(F)(F)F)nc(OC[C@@]56CCCN5C[C@H](F)C6)nc4c3C)c12 ; CCc1cccc2cc(O)cc(-c3nc(CC)c4c(N5CC6CCC(C5)N6C(=O)C(F)(F)F)nc(OC[C@@]56CCCN5C[C@H](F)C6)nc4c3F)c12 ; O=C(N1C2CCC1CN(c1nc(OC[C@@]34CCCN3C[C@H](F)C4)nc3c(Cl)c(-c4cc(O)cc5cccc(Cl)c45)nc(Cl)c13)C2)C(F)(F)F ; Cc1nc(-c2cc(O)cc3ccc(F)c(Cl)c23)c(F)c2nc(OC[C@@]34CCCN3C[C@H](F)C4)nc(N3CC4CCC(C3)N4C(=O)C(F)(F)F)c12]

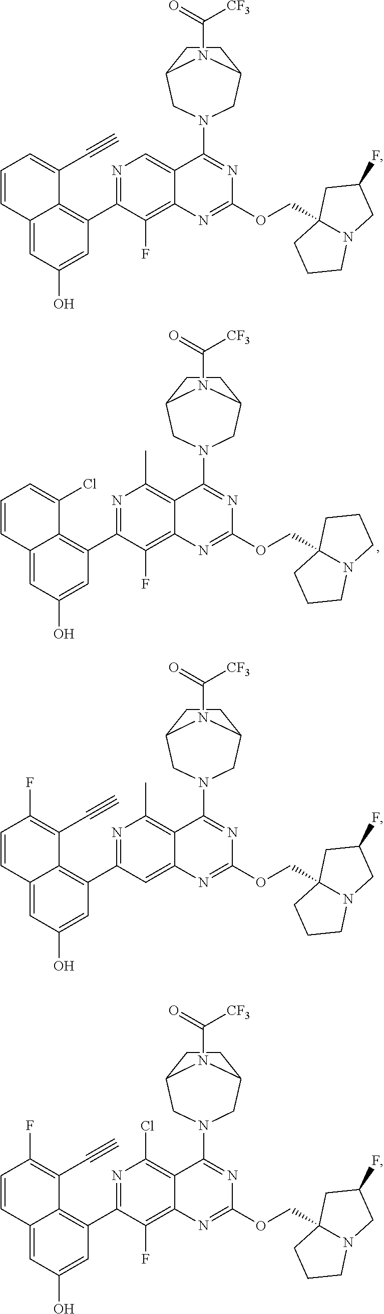

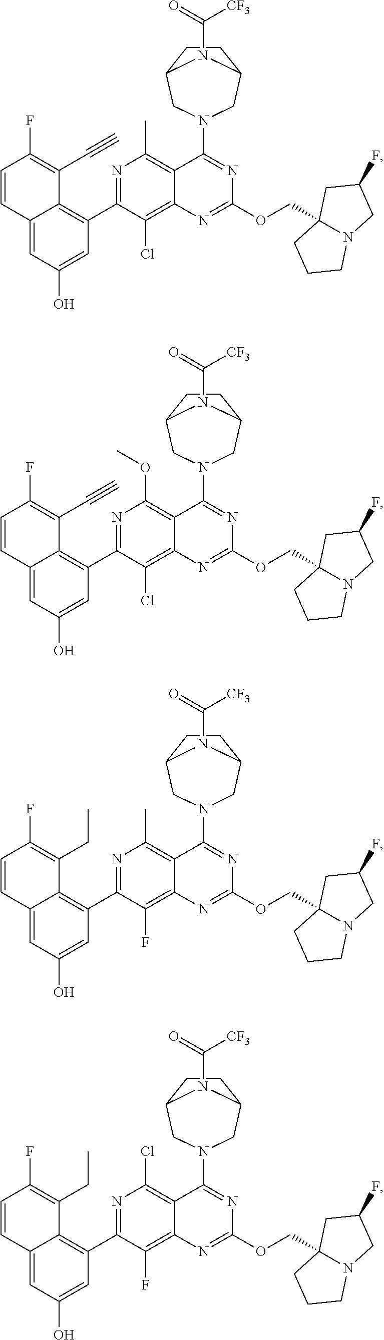 [SMILES: C#Cc1c(F)ccc2cc(O)cc(-c3nc(C)c4c(N5CC6CCC(C5)N6C(=O)C(F)(F)F)nc(OC[C@@]56CCCN5C[C@H](F)C6)nc4c3Cl)c12 ; C#Cc1c(F)ccc2cc(O)cc(-c3nc(OC)c4c(N5CC6CCC(C5)N6C(=O)C(F)(F)F)nc(OC[C@@]56CCCN5C[C@H](F)C6)nc4c3Cl)c12 ; CCc1c(F)ccc2cc(O)cc(-c3nc(C)c4c(N5CC6CCC(C5)N6C(=O)C(F)(F)F)nc(OC[C@@]56CCCN5C[C@H](F)C6)nc4c3F)c12 ; CCc1c(F)ccc2cc(O)cc(-c3nc(Cl)c4c(N5CC6CCC(C5)N6C(=O)C(F)(F)F)nc(OC[C@@]56CCCN5C[C@H](F)C6)nc4c3F)c12]

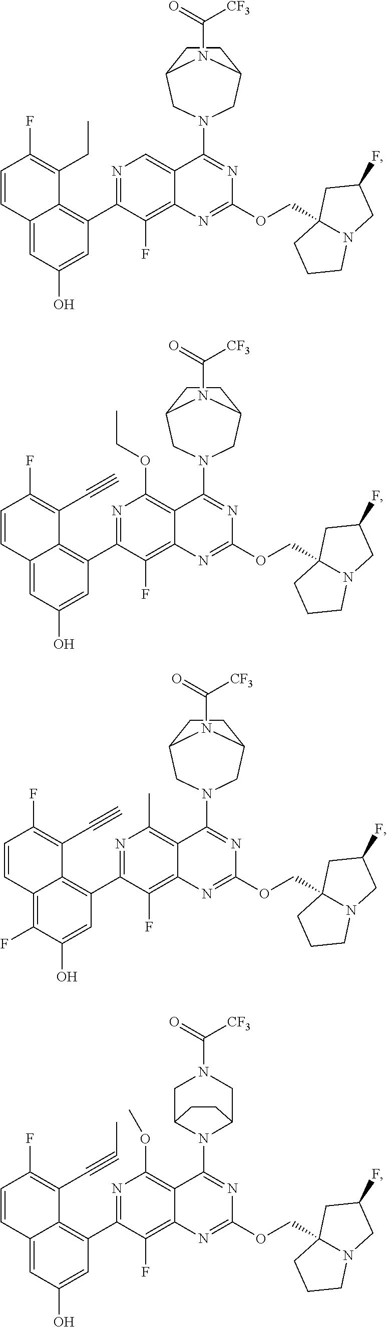 [SMILES: CCc1c(F)ccc2cc(O)cc(-c3ncc4c(N5CC6CCC(C5)N6C(=O)C(F)(F)F)nc(OC[C@@]56CCCN5C[C@H](F)C6)nc4c3F)c12 ; C#Cc1c(F)ccc2cc(O)cc(-c3nc(OCC)c4c(N5CC6CCC(C5)N6C(=O)C(F)(F)F)nc(OC[C@@]56CCCN5C[C@H](F)C6)nc4c3F)c12 ; C#Cc1c(F)ccc2c(F)c(O)cc(-c3nc(C)c4c(N5CC6CCC(C5)N6C(=O)C(F)(F)F)nc(OC[C@@]56CCCN5C[C@H](F)C6)nc4c3F)c12 ; CC#Cc1c(F)ccc2cc(O)cc(-c3nc(OC)c4c(N5C6CCC5CN(C(=O)C(F)(F)F)C6)nc(OC[C@@]56CCCN5C[C@H](F)C6)nc4c3F)c12]

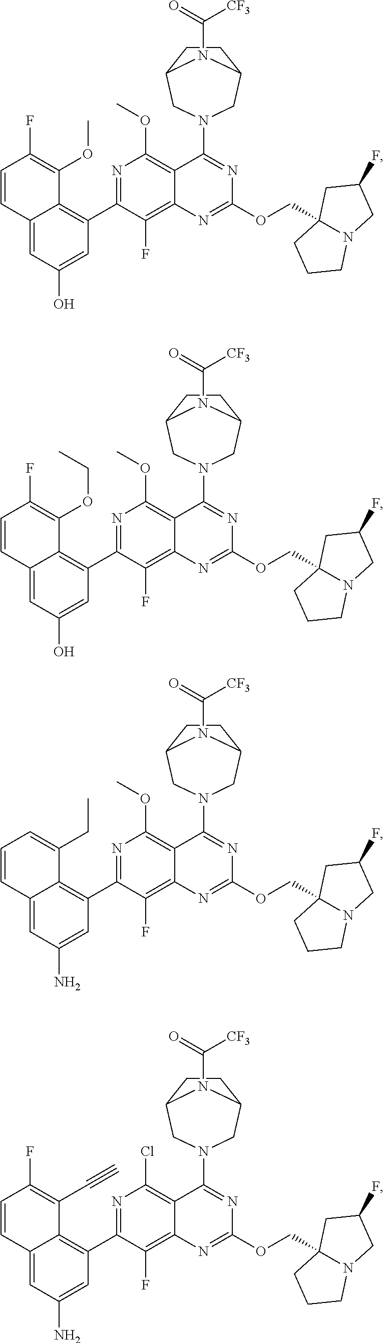 [SMILES: COc1c(F)ccc2cc(O)cc(-c3nc(OC)c4c(N5CC6CCC(C5)N6C(=O)C(F)(F)F)nc(OC[C@@]56CCCN5C[C@H](F)C6)nc4c3F)c12 ; CCOc1c(F)ccc2cc(O)cc(-c3nc(OC)c4c(N5CC6CCC(C5)N6C(=O)C(F)(F)F)nc(OC[C@@]56CCCN5C[C@H](F)C6)nc4c3F)c12 ; CCc1cccc2cc(N)cc(-c3nc(OC)c4c(N5CC6CCC(C5)N6C(=O)C(F)(F)F)nc(OC[C@@]56CCCN5C[C@H](F)C6)nc4c3F)c12 ; C#Cc1c(F)ccc2cc(N)cc(-c3nc(Cl)c4c(N5CC6CCC(C5)N6C(=O)C(F)(F)F)nc(OC[C@@]56CCCN5C[C@H](F)C6)nc4c3F)c12]

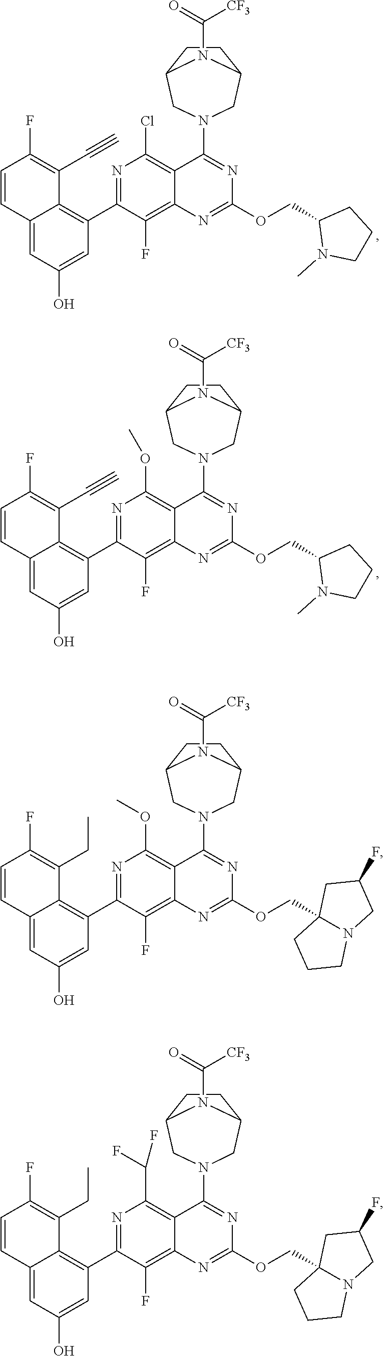 [SMILES: C#Cc1c(F)ccc2cc(O)cc(-c3nc(Cl)c4c(N5CC6CCC(C5)N6C(=O)C(F)(F)F)nc(OC[C@@H]5CCCN5C)nc4c3F)c12 ; C#Cc1c(F)ccc2cc(O)cc(-c3nc(OC)c4c(N5CC6CCC(C5)N6C(=O)C(F)(F)F)nc(OC[C@@H]5CCCN5C)nc4c3F)c12 ; CCc1c(F)ccc2cc(O)cc(-c3nc(OC)c4c(N5CC6CCC(C5)N6C(=O)C(F)(F)F)nc(OC[C@@]56CCCN5C[C@H](F)C6)nc4c3F)c12 ; CCc1c(F)ccc2cc(O)cc(-c3nc(C(F)F)c4c(N5CC6CCC(C5)N6C(=O)C(F)(F)F)nc(OC[C@@]56CCCN5C[C@H](F)C6)nc4c3F)c12]

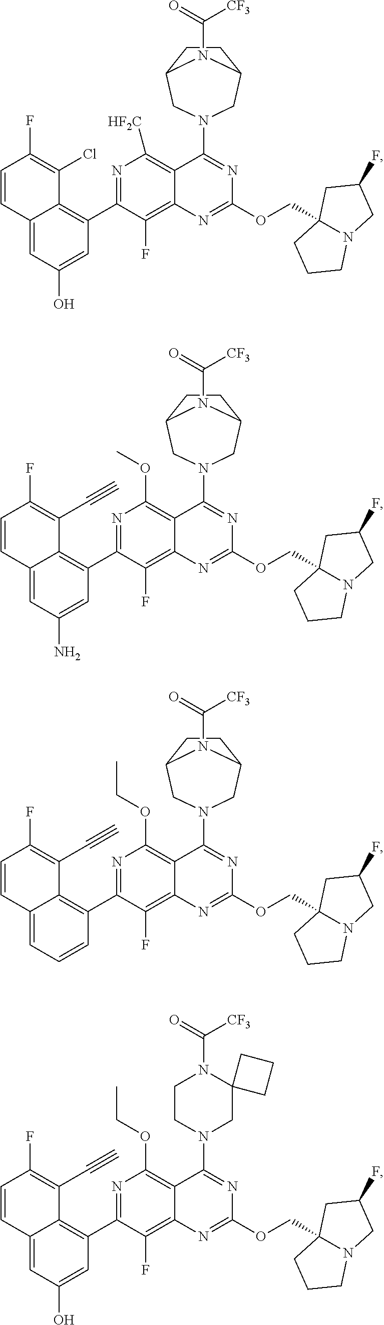

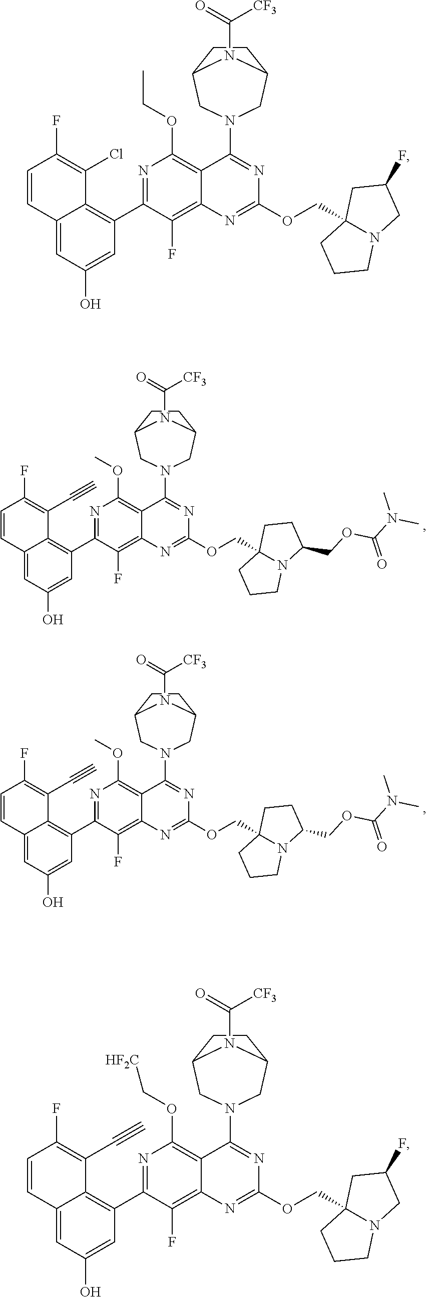

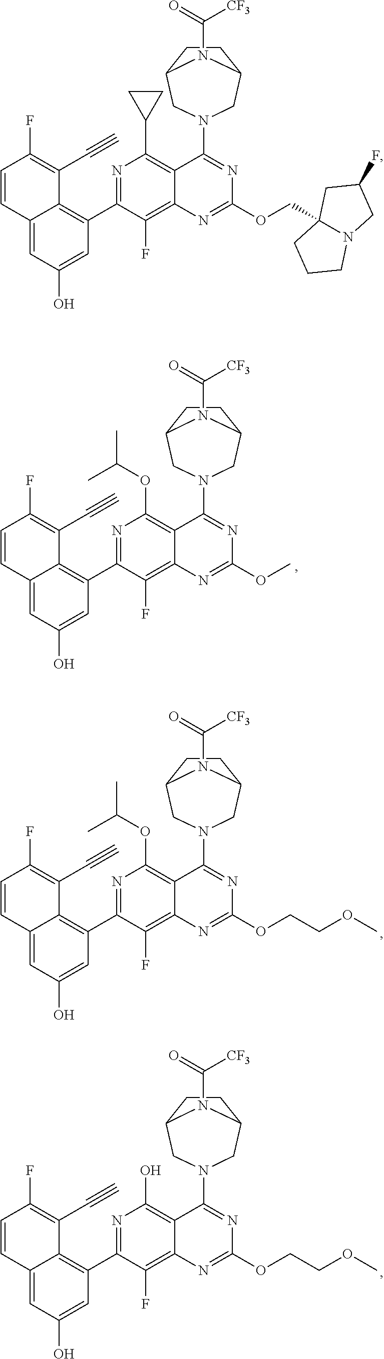 [SMILES: C#Cc1c(F)ccc2cc(O)cc(-c3nc(C4CC4)c4c(N5CC6CCC(C5)N6C(=O)C(F)(F)F)nc(OC[C@@]56CCCN5C[C@H](F)C6)nc4c3F)c12 ; C#Cc1c(F)ccc2cc(O)cc(-c3nc(OC(C)C)c4c(N5CC6CCC(C5)N6C(=O)C(F)(F)F)nc(OC)nc4c3F)c12 ; C#Cc1c(F)ccc2cc(O)cc(-c3nc(OC(C)C)c4c(N5CC6CCC(C5)N6C(=O)C(F)(F)F)nc(OCCOC)nc4c3F)c12 ; C#Cc1c(F)ccc2cc(O)cc(-c3nc(O)c4c(N5CC6CCC(C5)N6C(=O)C(F)(F)F)nc(OCCOC)nc4c3F)c12]

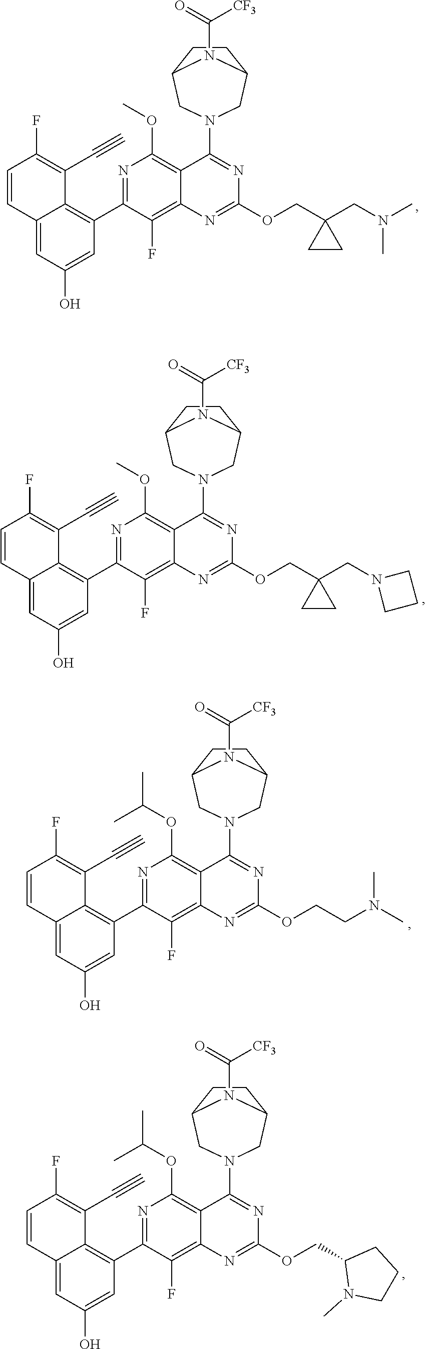 [SMILES: C#Cc1c(F)ccc2cc(O)cc(-c3nc(OC)c4c(N5CC6CCC(C5)N6C(=O)C(F)(F)F)nc(OCC5(CN(C)C)CC5)nc4c3F)c12 ; C#Cc1c(F)ccc2cc(O)cc(-c3nc(OC)c4c(N5CC6CCC(C5)N6C(=O)C(F)(F)F)nc(OCC5(CN6CCC6)CC5)nc4c3F)c12 ; C#Cc1c(F)ccc2cc(O)cc(-c3nc(OC(C)C)c4c(N5CC6CCC(C5)N6C(=O)C(F)(F)F)nc(OCCN(C)C)nc4c3F)c12 ; C#Cc1c(F)ccc2cc(O)cc(-c3nc(OC(C)C)c4c(N5CC6CCC(C5)N6C(=O)C(F)(F)F)nc(OC[C@@H]5CCCN5C)nc4c3F)c12]

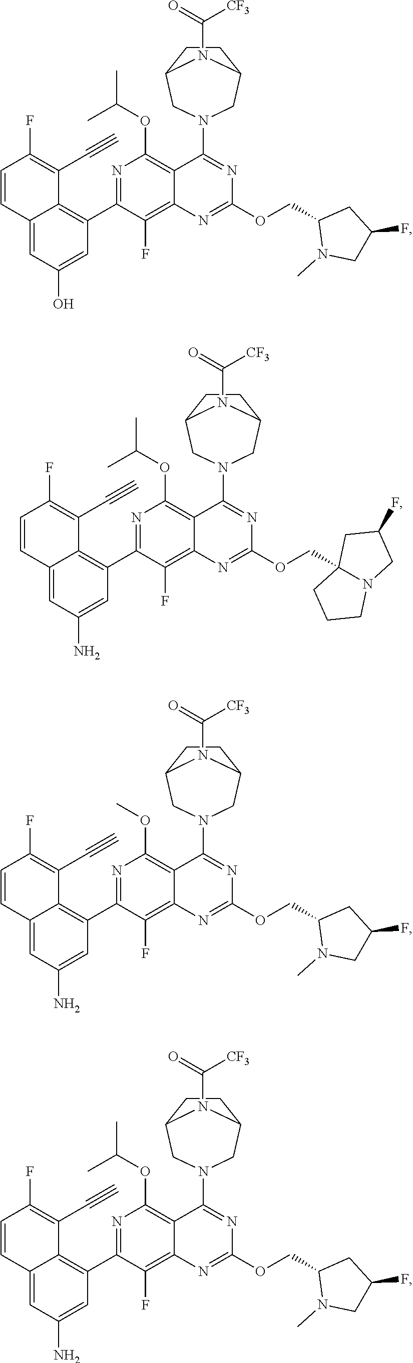 [SMILES: C#Cc1c(F)ccc2cc(O)cc(-c3nc(OC(C)C)c4c(N5CC6CCC(C5)N6C(=O)C(F)(F)F)nc(OC[C@@H]5C[C@@H](F)CN5C)nc4c3F)c12 ; C#Cc1c(F)ccc2cc(N)cc(-c3nc(OC(C)C)c4c(N5CC6CCC(C5)N6C(=O)C(F)(F)F)nc(OC[C@@]56CCCN5C[C@H](F)C6)nc4c3F)c12 ; C#Cc1c(F)ccc2cc(N)cc(-c3nc(OC)c4c(N5CC6CCC(C5)N6C(=O)C(F)(F)F)nc(OC[C@@H]5C[C@@H](F)CN5C)nc4c3F)c12 ; C#Cc1c(F)ccc2cc(N)cc(-c3nc(OC(C)C)c4c(N5CC6CCC(C5)N6C(=O)C(F)(F)F)nc(OC[C@@H]5C[C@@H](F)CN5C)nc4c3F)c12]

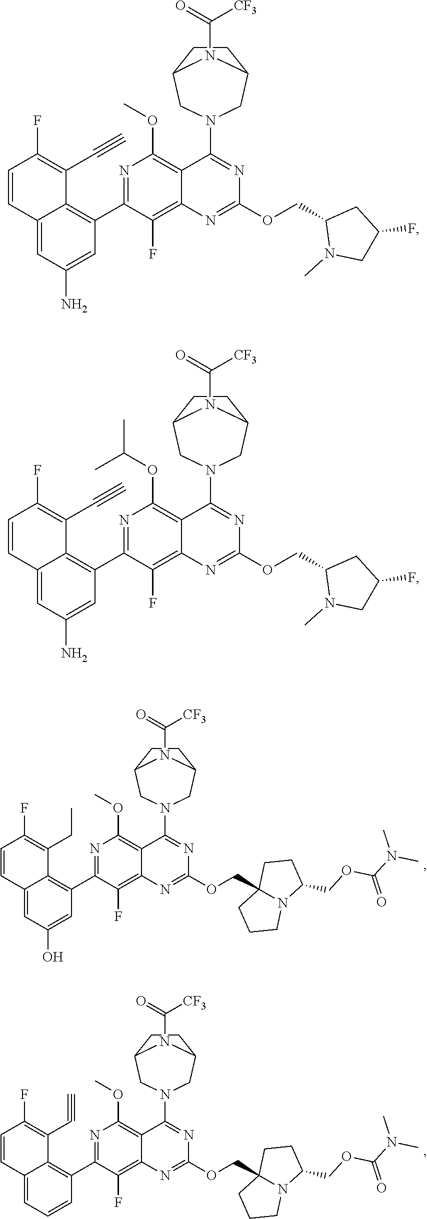 [SMILES: C#Cc1c(F)ccc2cc(N)cc(-c3nc(OC)c4c(N5CC6CCC(C5)N6C(=O)C(F)(F)F)nc(OC[C@@H]5C[C@H](F)CN5C)nc4c3F)c12 ; C#Cc1c(F)ccc2cc(N)cc(-c3nc(OC(C)C)c4c(N5CC6CCC(C5)N6C(=O)C(F)(F)F)nc(OC[C@@H]5C[C@H](F)CN5C)nc4c3F)c12 ; CCc1c(F)ccc2cc(O)cc(-c3nc(OC)c4c(N5CC6CCC(C5)N6C(=O)C(F)(F)F)nc(OC[C@]56CCCN5[C@@H](COC(=O)N(C)C)CC6)nc4c3F)c12 ; C#Cc1c(F)ccc2cccc(-c3nc(OC)c4c(N5CC6CCC(C5)N6C(=O)C(F)(F)F)nc(OC[C@]56CCCN5[C@@H](COC(=O)N(C)C)CC6)nc4c3F)c12]

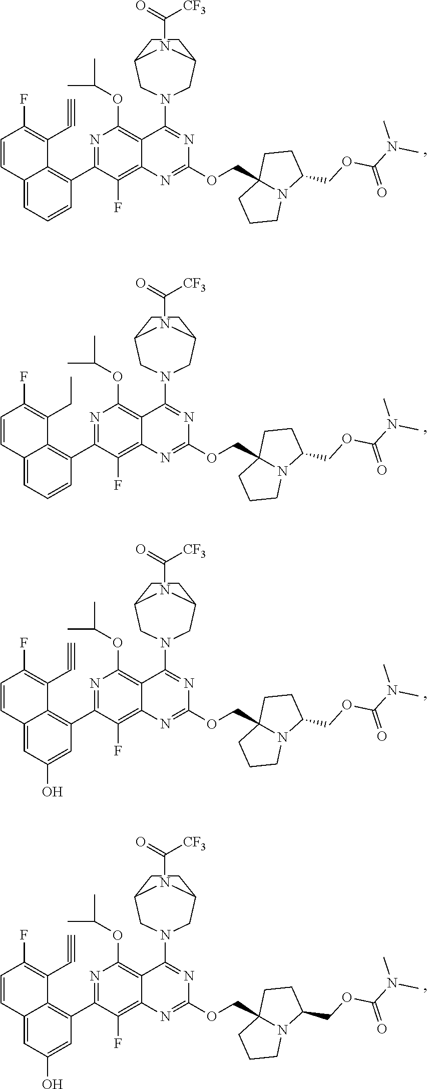 [SMILES: C#Cc1c(F)ccc2cccc(-c3nc(OC(C)C)c4c(N5CC6CCC(C5)N6C(=O)C(F)(F)F)nc(OC[C@]56CCCN5[C@@H](COC(=O)N(C)C)CC6)nc4c3F)c12 ; CCc1c(F)ccc2cccc(-c3nc(OC(C)C)c4c(N5CC6CCC(C5)N6C(=O)C(F)(F)F)nc(OC[C@]56CCCN5[C@@H](COC(=O)N(C)C)CC6)nc4c3F)c12 ; C#Cc1c(F)ccc2cc(O)cc(-c3nc(OC(C)C)c4c(N5CC6CCC(C5)N6C(=O)C(F)(F)F)nc(OC[C@]56CCCN5[C@@H](COC(=O)N(C)C)CC6)nc4c3F)c12 ; C#Cc1c(F)ccc2cc(O)cc(-c3nc(OC(C)C)c4c(N5CC6CCC(C5)N6C(=O)C(F)(F)F)nc(OC[C@]56CCCN5[C@H](COC(=O)N(C)C)CC6)nc4c3F)c12]

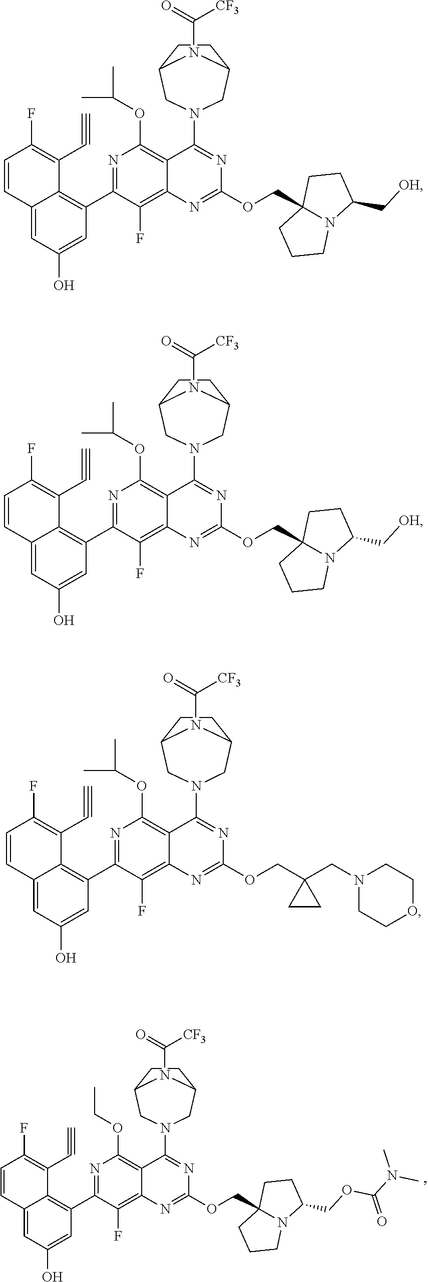 [SMILES: C#Cc1c(F)ccc2cc(O)cc(-c3nc(OC(C)C)c4c(N5CC6CCC(C5)N6C(=O)C(F)(F)F)nc(OC[C@]56CCCN5[C@H](CO)CC6)nc4c3F)c12 ; C#Cc1c(F)ccc2cc(O)cc(-c3nc(OC(C)C)c4c(N5CC6CCC(C5)N6C(=O)C(F)(F)F)nc(OC[C@]56CCCN5[C@@H](CO)CC6)nc4c3F)c12 ; C#Cc1c(F)ccc2cc(O)cc(-c3nc(OC(C)C)c4c(N5CC6CCC(C5)N6C(=O)C(F)(F)F)nc(OCC5(CN6CCOCC6)CC5)nc4c3F)c12 ; C#Cc1c(F)ccc2cc(O)cc(-c3nc(OCC)c4c(N5CC6CCC(C5)N6C(=O)C(F)(F)F)nc(OC[C@]56CCCN5[C@@H](COC(=O)N(C)C)CC6)nc4c3F)c12]

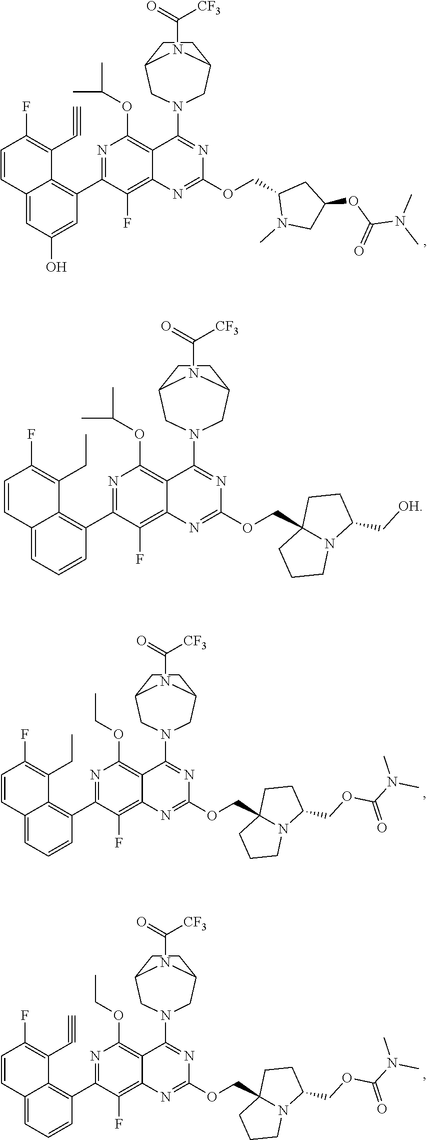 [SMILES: C#Cc1c(F)ccc2cc(O)cc(-c3nc(OC(C)C)c4c(N5CC6CCC(C5)N6C(=O)C(F)(F)F)nc(OC[C@@H]5C[C@@H](OC(=O)N(C)C)CN5C)nc4c3F)c12 ; CCc1c(F)ccc2cccc(-c3nc(OC(C)C)c4c(N5CC6CCC(C5)N6C(=O)C(F)(F)F)nc(OC[C@]56CCCN5[C@@H](CO)CC6)nc4c3F)c12 ; CCOc1nc(-c2cccc3ccc(F)c(CC)c23)c(F)c2nc(OC[C@]34CCCN3[C@@H](COC(=O)N(C)C)CC4)nc(N3CC4CCC(C3)N4C(=O)C(F)(F)F)c12 ; C#Cc1c(F)ccc2cccc(-c3nc(OCC)c4c(N5CC6CCC(C5)N6C(=O)C(F)(F)F)nc(OC[C@]56CCCN5[C@@H](COC(=O)N(C)C)CC6)nc4c3F)c12]

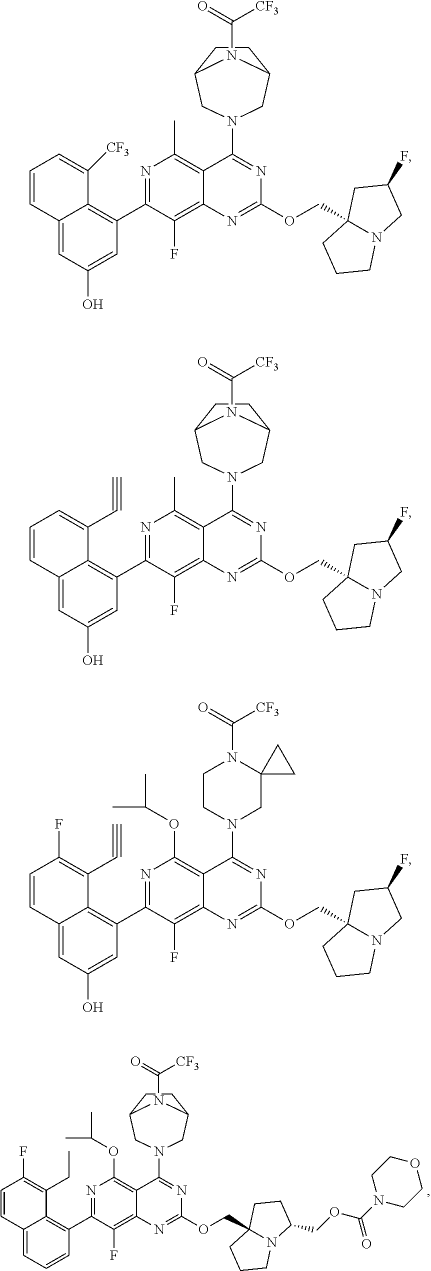 [SMILES: Cc1nc(-c2cc(O)cc3cccc(C(F)(F)F)c23)c(F)c2nc(OC[C@@]34CCCN3C[C@H](F)C4)nc(N3CC4CCC(C3)N4C(=O)C(F)(F)F)c12 ; C#Cc1cccc2cc(O)cc(-c3nc(C)c4c(N5CC6CCC(C5)N6C(=O)C(F)(F)F)nc(OC[C@@]56CCCN5C[C@H](F)C6)nc4c3F)c12 ; C#Cc1c(F)ccc2cc(O)cc(-c3nc(OC(C)C)c4c(N5CCN(C(=O)C(F)(F)F)C6(CC6)C5)nc(OC[C@@]56CCCN5C[C@H](F)C6)nc4c3F)c12 ; CCc1c(F)ccc2cccc(-c3nc(OC(C)C)c4c(N5CC6CCC(C5)N6C(=O)C(F)(F)F)nc(OC[C@]56CCCN5[C@@H](COC(=O)N5CCOCC5)CC6)nc4c3F)c12]

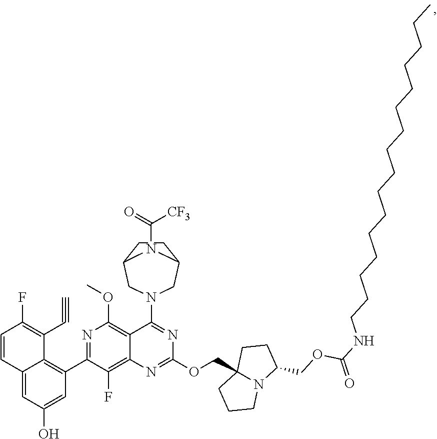 [SMILES: C#Cc1c(F)ccc2cc(O)cc(-c3nc(OC)c4c(N5CC6CCC(C5)N6C(=O)C(F)(F)F)nc(OC[C@]56CCCN5[C@@H](COC(=O)NCCCCCCCCCCCCCCCC)CC6)nc4c3F)c12]

### Biological Assays/Testing

Cell lines

The following cancer cell lines were employed: A427 human lung carcinoma [1 copy of KRAS(G12D)] (ATCC, #HTB-53); AGS human gastric adenocarcinoma [1 copy of KRAS(G12D)] (ATCC, #CRL-1739); and AsPC-1 human adenocarcinoma [2 copies of KRAS(G12D)] (ATCC, CRL-1682). Cell lines were cultured essentially according to American Type Culture Collection (ATCC) recommendations.

### Cell Cytotoxicity

A427 and AGS cells were plated in 96-well tissue culture plates at 4,000 cells/well and incubated at 37° C/5% CO2 for 72 hr in 100 μl of media. 3-fold serial dilutions of each test compound were prepared ranging from 20 μM to 1.02 nM. Each cell line was then treated with test compounds at various concentrations with a final concentration of 0.5%

DMSO/well, and then incubated at 37° C/5% CO2 for 24 hr. 100 Ill of CellTiter-Glo® Reagent (Promega Corporation, Madison, WI) was added to each well and processed according manufacturer's protocol. Results were analyzed and IC50 values were calculated in GraphPad 7 software. Results are listed in Table 1.

### KRAS(GI2D)/SOSI Homogeneous Time-Resolved Fluorescence (HTRF) Assay

Binding of test compounds to KRAS(G12D) target protein, which in turn blocks KRAS(G12D) interaction with the SOS1 protein, was measured in the absences of GTP by homogeneous time-resolved fluorescence using the KRAS-G12D/SOS1 Binding Assay Kit (Cisbio, #63ADK000CB16PEG), following the manufacturer's instructions, except as noted. 3-fold serial dilutions of each test compound were prepared ranging from 20 μM to 1.02 nM. The test compound was mixed and incubated with reaction components, incubated in a sealed plate at 4° C. for 3 hr and fluorescence was measured using a PerkinElmer Envision plate reader. The KRAS(G12D)-SOS1 IC50 values (the concentration of 50% of the maximal inhibition) were calculated using GraphPad Prism 7 software. Results are listed in Table 1.

TABLE 1
Cytotoxicity and Target Binding Assays

| | ¹A427 72H | ¹AGS 72H | ²KRAS(G12D)- |
|---|---|---|---|
| Compound | CTG | CTG | SOS1 HTRF |
| 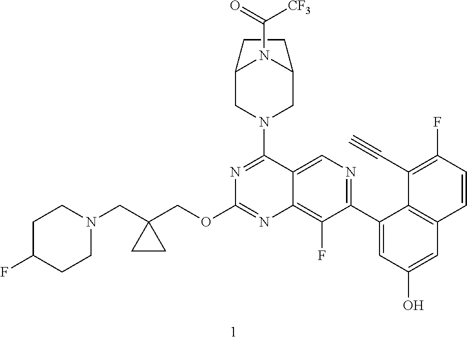 [SMILES: C#Cc1c(F)ccc2cc(O)cc(-c3ncc4c(N5CC6CCC(C5)N6C(=O)C(F)(F)F)nc(OCC5(CN6CCC(F)CC6)CC5)nc4c3F)c12] | A | A | A |
| 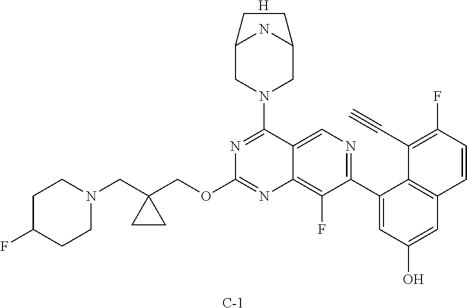 | A | A | |
| 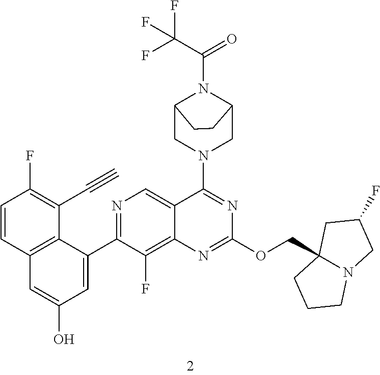 [SMILES: C#Cc1c(F)ccc2cc(O)cc(-c3ncc4c(N5CC6CCC(C5)N6C(=O)C(F)(F)F)nc(OC[C@]56CCCN5C[C@@H](F)C6)nc4c3F)c12] | C | A | |
| 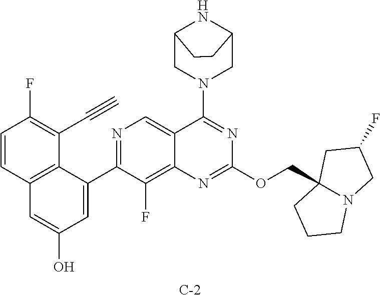 | | | |
| 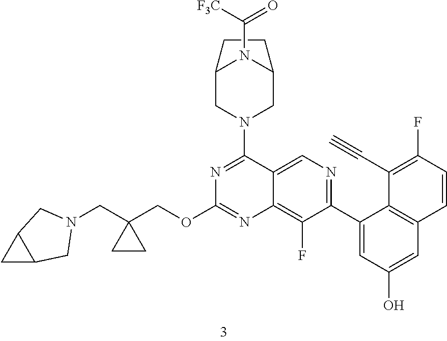 [SMILES: C#Cc1c(F)ccc2cc(O)cc(-c3ncc4c(N5CC6CCC(C5)N6C(=O)C(F)(F)F)nc(OCC5(CN6CC7CC7C6)CC5)nc4c3F)c12] | C | A | |
| 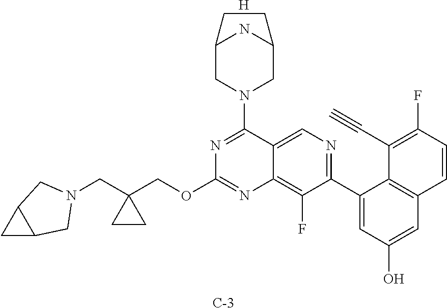 | A | A | A |

¹CellTiter-Glo cytotoxicity assay for A427 and AGS cells: A: 1-100 nM, B: 101-1000 nM, C: 1001-10000 nM, D: >10,000 nM;
²KRAS(G12D)-SOS1 HTRF binding. A: 1-100 nM, B: 101-1000 nM, C: 1001-10000 nM, D: >10,000 nM.

### Oral Bioavailability

CD-1 mice were randomly assigned to δgroups with 3 male mice in each compound group. Control groups included Compound 1 (50 mg/kg), Compound 3 (25 mg/kg), and Compound 5 (25 mg/kg). Trifluoroacetate (COCF3) groups included Compound 2 (50 mg/kg), Compound 4 (50 mg/kg) and Compound 6 (50 mg/kg). Compounds were orally administered (PO) in a single dose to each mouse in its group. Blood samples were taken within 72 hours. Bioavailability (F%) was determined and by liquid chromatography-mass spectrometry (LC-MS/MS). The mean oral %F is provided in Table 2.

TABLE 2

| Compound | % F |
|---|---|
| 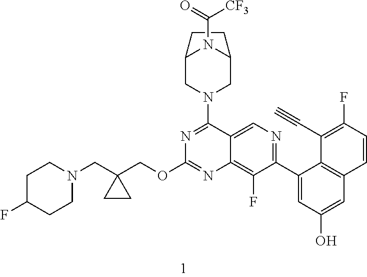 [SMILES: C#Cc1c(F)ccc2cc(O)cc(-c3ncc4c(N5CC6CCC(C5)N6C(=O)C(F)(F)F)nc(OCC5(CN6CCC(F)CC6)CC5)nc4c3F)c12] | 12.4 |
| 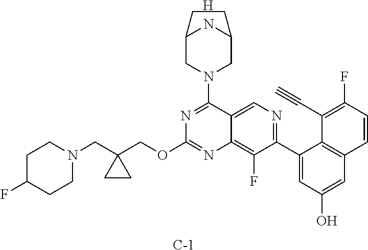 | 0.82 |
| 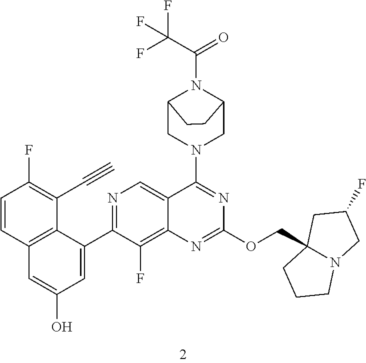 [SMILES: C#Cc1c(F)ccc2cc(O)cc(-c3ncc4c(N5CC6CCC(C5)N6C(=O)C(F)(F)F)nc(OC[C@]56CCCN5C[C@@H](F)C6)nc4c3F)c12] | 15.8 |
| 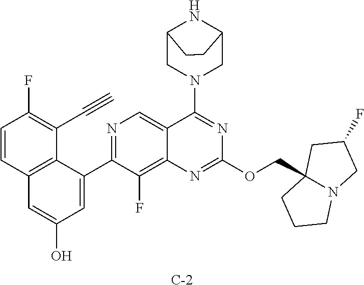 | 0.45 |
| 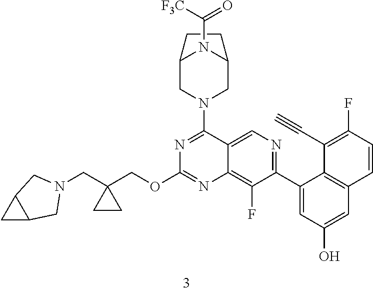 [SMILES: C#Cc1c(F)ccc2cc(O)cc(-c3ncc4c(N5CC6CCC(C5)N6C(=O)C(F)(F)F)nc(OCC5(CN6CC7CC7C6)CC5)nc4c3F)c12] | 17.4 |
| 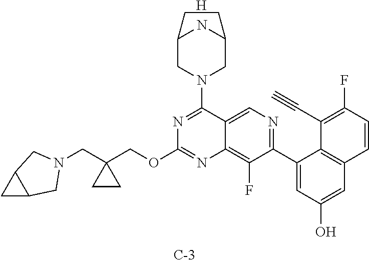 | 0.47 |

### In Vivo Xenograft Tumor Model

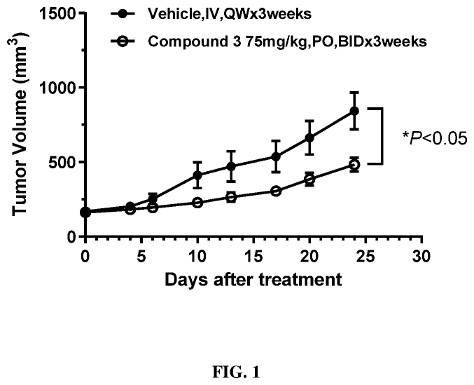

BALB/c nude mice at 8 to 9 weeks age were maintained in a pathogen-free environment. AsPC-1 cells (5×10⁶) were subcutaneously implanted into female mice. Mice bearing established tumors (˜170 mm3) were randomized into treatment groups of 4. Mice were intravenously (i.v.) dosed with 5% sulfobutylether-beta-cyclodextrin (SBECD) vehicle control once a week (QW) or orally dosed (PO) twice a day (BID) with 75 mg/kg Compound 3, for over 3 weeks. Tumors were measured twice a week with calipers. Tumor volume (TV) was calculated as TV=(L×W2)/2, where L is tumor length and W is tumor width. Results were graphed with +/− standard error (SEM). Student T-test with Bonferroni post hoc adjustment was used to measure statistical significance of the final (day 24) tumor measurements (P=0.036). Results are shown in FIG. 1.

Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

All patents and publications mentioned in this specification are herein incorporated by reference to the same extent as if each independent patent and publication was specifically and individually indicated to be incorporated by reference.

1. A compound of the structural formula I*:
R⁰⁰F₂C—L—R⁰ (I*);
or a pharmaceutically acceptable salt thereof, wherein
L is selected from —C(═O), —S(═O), and —S(O)₂;
R⁰ is a chemical entity which binds to KRASG12D; and
R⁰⁰ is selected from hydrogen, F, Cl, and CF₃.

2. (canceled)

3. The compound of claim 1, wherein the compound is of the structural formula Ia or Ib: 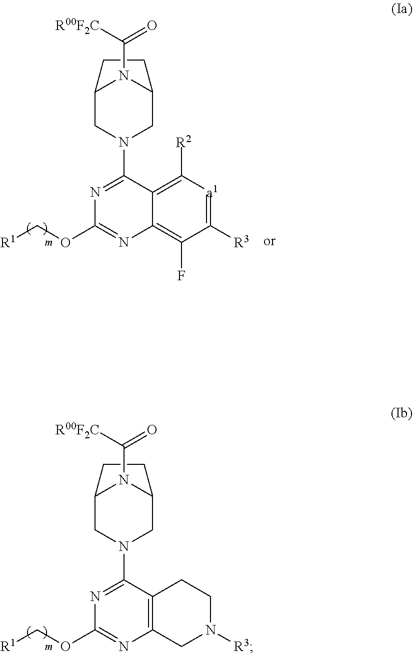
or a pharmaceutically acceptable salt thereof, wherein
R¹ is selected from (C₁-C₄)alkylO(C₁-C₄)alkyl, -(C₁-C₄)alkylNH(C₁-C₄)alkyl, -(C₁-C₄)alkylN[(C₁-C₄)alkyl]₂, heterocyclyl, and cycloalkyl, wherein said heterocyclyl and cycloalkyl are each optionally and independently substituted;
R² is selected from hydrogen, (C₁-C₄)alkyl, (C₁-C₄)haloalkyl, (C₁-C₄)alkoxy, (C₁-C₄)haloalkoxy, (C₁-C₄)alkynyl, (C₁-C₄)alkenyl, halo, (C₃-C₆)cycloalkyl, -O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl;
R³ is selected from 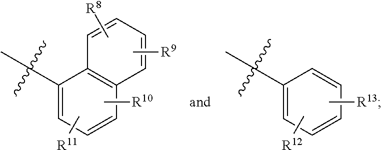
a¹ is N or CHZ1;
Z1 is selected from hydrogen, halo, (C₁-C₄)alkyl, (C₂-C₄)alkenyl, cyano, cyano(C₁-C₄)alkyl, —S[halo(C₁-C₄)alkyl], and (C₃-C₆)cycloalkyl;
R⁸, R⁹, R¹⁰, R¹¹, R¹², and R¹³ are each independently selected from hydrogen, (C₁-C₄)alkyl, (C₁-C₄)haloalkyl, (C₁-C₄)cyanoalkyl, (C₁-C₄)hydroxyalkyl, -(C₁-C₄)alkylNR^{a}R^{b}, -(C₁-C₄)alkylC(O)NR^{a}R^{b}, (C₁-C₄)alkylO(C₁-C₄)alkyl, -(C₁-C₄)alkylC(O)OR^{a}, -(C₁-C₄)alkylN^{a}(O)OR^{b}, -(C₁-C₄)alkylC(O)R^{a}, -(C₁-C₄)alkylheterocyclyl, -(C₁-C₄)alkylaryl, -(C₁-C₄)alkylheteroaryl, (C₂-C₄)alkenyl, (C₂-C₄)haloalkenyl, (C₂-C₄)cyanoalkenyl, (C₂-C₄)hydroxyalkenyl, -(C₂-C₄)alkenylNR^{a}R^{b}, (C₂-C₄)alkynyl, (C₂-C₄)haloalkynyl, (C₂-C₄)cyanoalkynyl, (C₂-C₄)hydroxyalkynyl, -(C₂-C₄)alkynylNR^{a}R^{b}, (C₁-C₄)alkoxy, (C₁-C₄)haloalkoxy, halo, cyano, oxo, hydroxy, —S(C₁-C₄)alkyl, —S(C₁-C₄)haloalkyl, -NRaC(O)R^{b}, —C(O)R^{a}, —C(O)OR^{a}, —SO₂R^{a}, —S(O)R^{a}, —SO₂NR^{a}R^{b}, —NRaSO₂R^{b}, (C₃-C₆)cycloalkyl, 5- or 6-membered heteroaryl, and 4- to 6-membered heterocyclyl, wherein said heterocyclyl, aryl, and heteroaryl of -(C₁-C₄)alkylheterocyclyl, -(C₁-C₄)alkylaryl, and -(C₁-C₄)alkylheteroaryl, and said (C₃-C₆)cycloalkyl, 5- or 6-membered heteroaryl, and 4- to 6-membered heterocyclyl are each optionally and independently substituted with 1 to 3 groups selected from R^{c},
m is 0, 1, or 2;
IV and R^{b} are each independently selected from hydrogen, (C₁-C₄)alkyl, and (C₁-C₄)haloalkyl, or IV and R^{b}, when on the same nitrogen atom, may be taken together to form a heterocyclyl; and
R^{c} is selected from halo, (C₁-C₄)alkyl, (C₁-C₄)haloalkyl, (C₁-C₄)alkoxy, (C₁-C₄)haloalkoxy, cyano, hydroxyl, oxo, —C(O)OR^{a}, —C(O)R^{a}, —SO₂R^{a}, —S(O)R^{a}, —SO₂NR^{a}R^{b}, -NRaC(O)R^{b}, —NRaSO₂R^{b}, —NR^{a}R^{b}, and NO₂.

4-8. (canceled)

9. The compound of Claim 1, wherein the compound is of the structural formula Ia′: 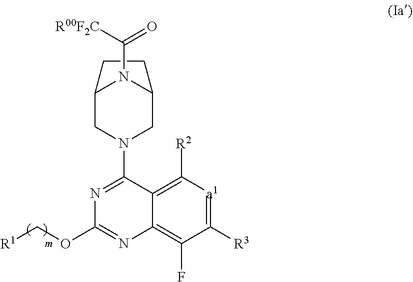
or a pharmaceutically acceptable salt thereof.

10. The compound of claim 9, or a pharmaceutically acceptable salt thereof, wherein al is N.

11. The compound of claim 9, or a pharmaceutically acceptable salt thereof, wherein R⁰⁰ is F.

12. The compound of claim 9, or a pharmaceutically acceptable salt thereof, wherein R³ is

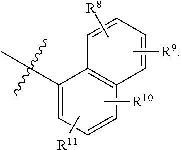

13. The compound of claim 9, or a pharmaceutically acceptable salt thereof, wherein R⁸, R⁹, R¹⁰, and R¹¹ are each independently selected from hydrogen, halo, hydroxyl, and (C₂-C₄)alkynyl.

14. The compound of claim 9, or a pharmaceutically acceptable salt thereof, wherein
R⁸, R⁹, R¹⁰, and R¹¹ are each hydrogen; or
R⁹, R¹¹ and R11 are each hydrogen and R⁸ is selected from halo, hydroxyl, and (C₂-C₄)alkynyl; or
R¹⁰ and R¹¹ are each hydrogen and R⁸ and R⁹ are each independently selected from halo, hydroxyl, and (C₂-C₄)alkynyl; or
R¹¹ is hydrogen and R⁸, R⁹, and R¹⁰ are each independently selected from halo, hydroxyl, and (C₂-C₄)alkynyl.

15. The compound of claim 9, or a pharmaceutically acceptable salt thereof, wherein
R⁸, R⁹, R¹⁰ and R¹¹ are each hydrogen; or
R⁹, R¹⁰ and R¹¹ are each hydrogen and R⁸ is (C₂-C₄)alkynyl; or
R¹⁰ and R¹¹ are each hydrogen, R⁸ is (C₂-C₄)alkynyl, and R⁹ is halo; or
R¹¹ is hydrogen, R⁸ is (C₂-C₄)alkynyl, R⁹ is halo, and R¹⁰ is hydroxyl.

16. The compound of claim 9, or a pharmaceutically acceptable salt thereof, wherein R³ is 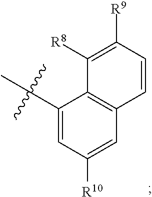
or a pharmaceutically acceptable salt thereof.

17. The compound of claim 9, or a pharmaceutically acceptable salt thereof, wherein R³ is 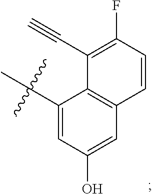 [SMILES: *c1cc(O)cc2ccc(F)c(C#C)c12]
or a pharmaceutically acceptable salt thereof.

18. The compound of claim 9, or a pharmaceutically acceptable salt thereof, wherein R³ is selected from

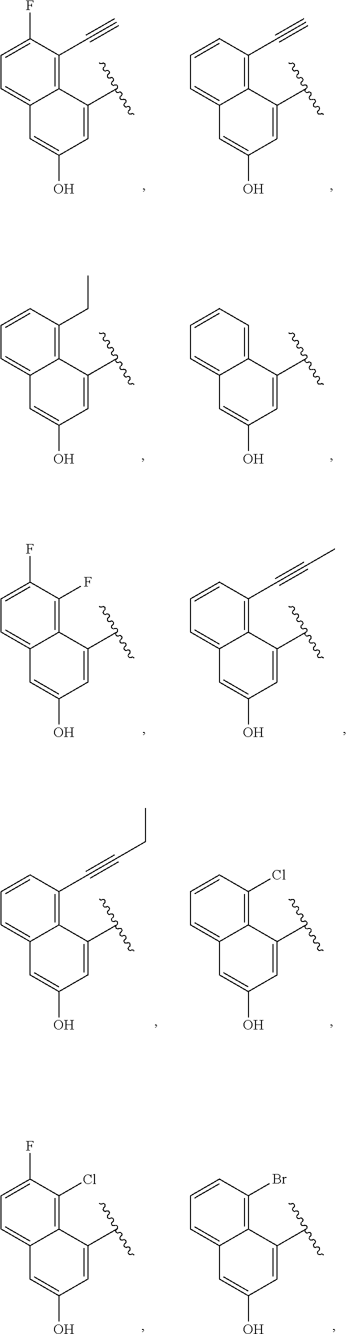

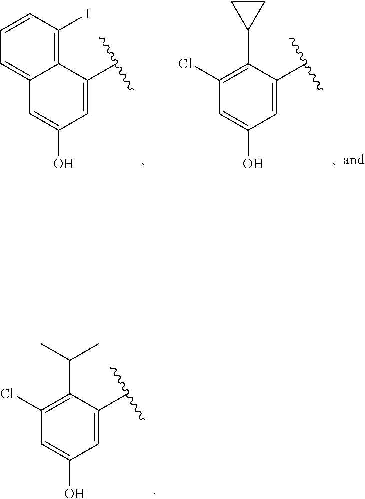 [SMILES: *c1cc(O)cc2cccc(I)c12 ; *c1cc(O)cc(Cl)c1C1CC1 ; *c1cc(O)cc(Cl)c1C(C)C]

19-23. (canceled)

24. The compound of claim 9, or a pharmaceutically acceptable salt thereof, wherein R¹ is

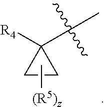

25. The compound of claim 9, or a pharmaceutically acceptable salt thereof, wherein R⁴ is -(C₁-C₄)alkylheterocyclyl optionally substituted with 1 to 3 groups selected from R^{c}.

26. (canceled)

27. The compound of claim 9, or a pharmaceutically acceptable salt thereof, wherein RC is selected from (C₁-C₄)alkyl, (C₁-C₄)haloalkyl, and halo.

28. (canceled)

29. The compound of claim 9, or a pharmaceutically acceptable salt thereof, wherein R¹ is selected from

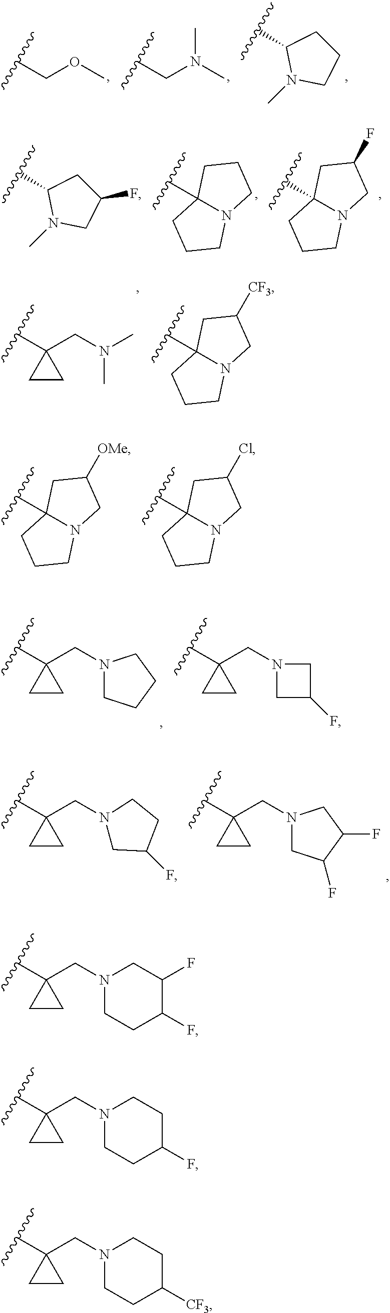

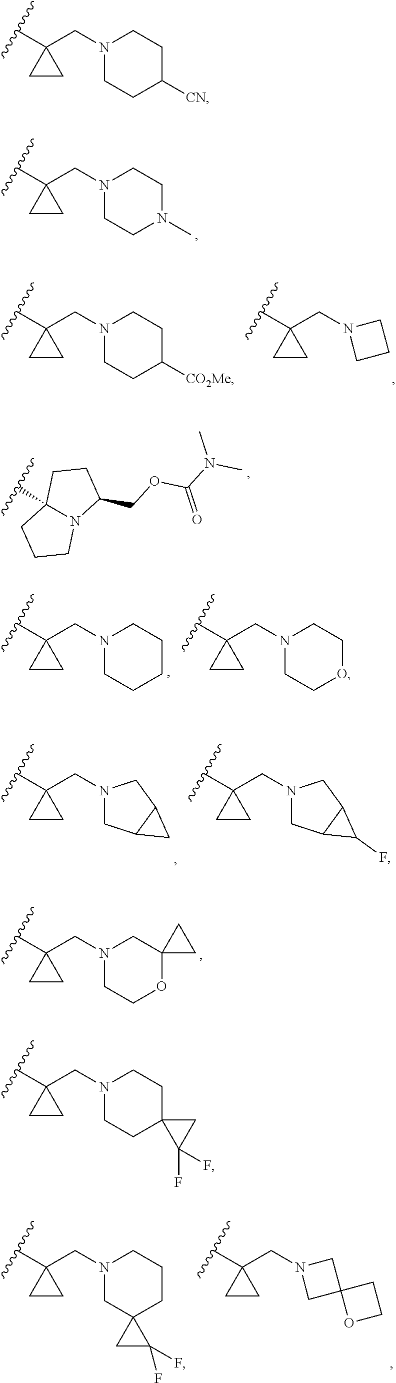

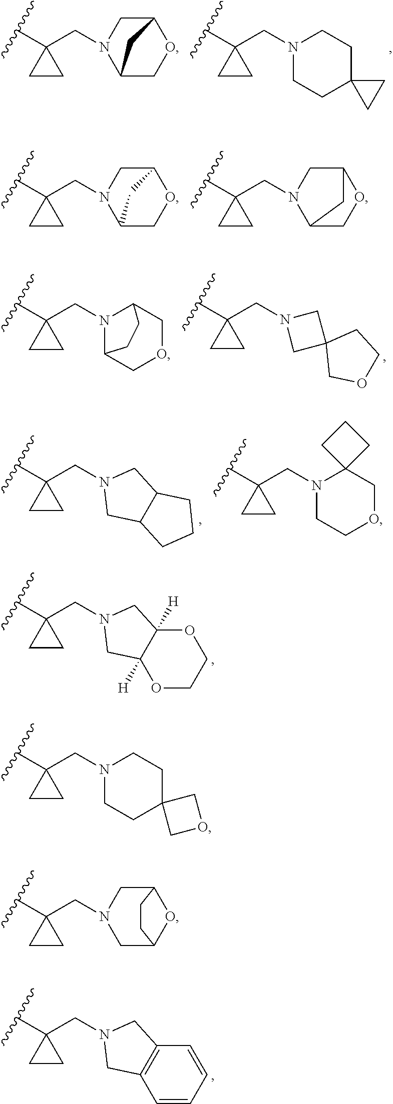

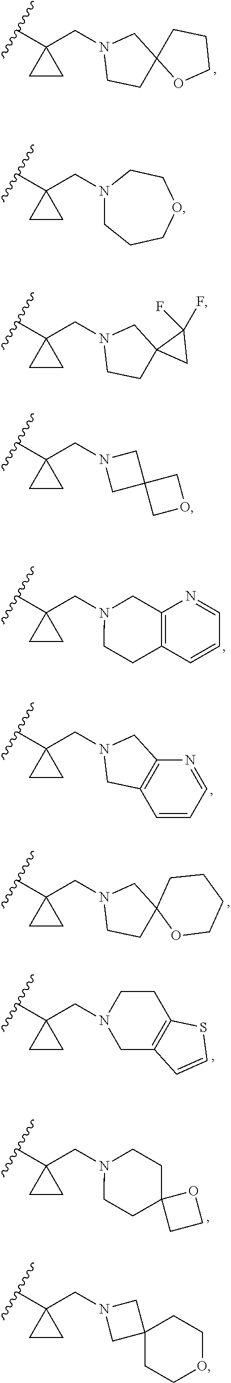

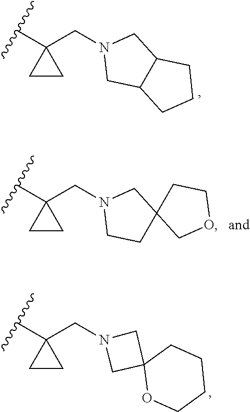 [SMILES: *C1(CN2CC3CCCC3C2)CC1 ; *C1(CN2CCC3(CCOC3)C2)CC1 ; "and" ; *C1(CN2CC3(CCCCO3)C2)CC1]

30. The compound of claim 9any one of claims 1 to 29, or a pharmaceutically acceptable salt thereof, wherein R² is selected from hydrogen, (C₁-C₄)alkyl, (C₁-C₄)haloalkyl, (C₁-C₄)alkoxy, (C₁-C₄)haloalkoxy, (C₁-C₄)alkynyl, (C₁-C₄)alkenyl, halo, (C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl.

31. (canceled)

32. (canceled)

33. The compound of claim 1, wherein the compound is selected from 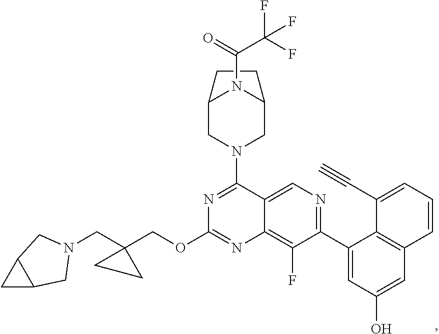 [SMILES: C#Cc1cccc2cc(O)cc(-c3ncc4c(N5CC6CCC(C5)N6C(=O)C(F)(F)F)nc(OCC5(CN6CC7CC7C6)CC5)nc4c3F)c12] 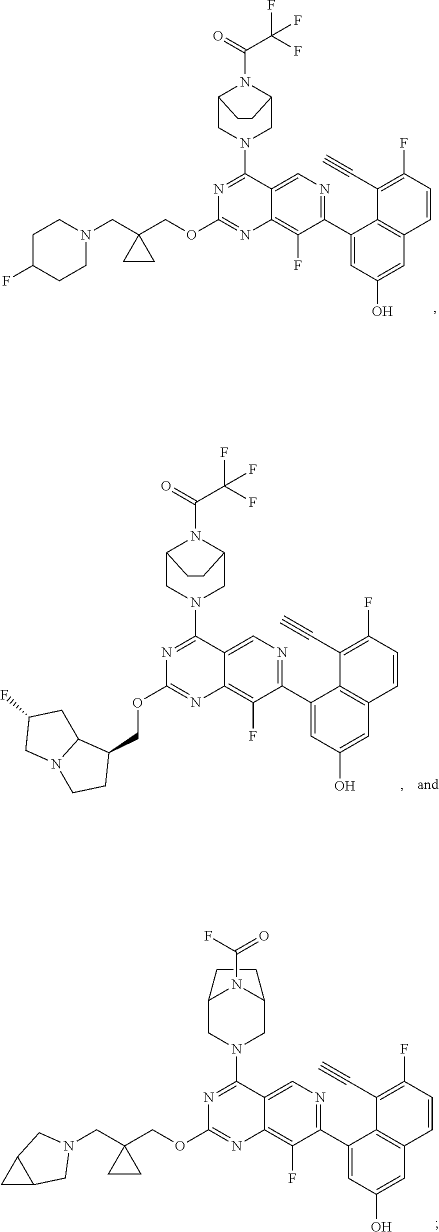 [SMILES: C#Cc1c(F)ccc2cc(O)cc(-c3ncc4c(N5CC6CCC(C5)N6C(=O)C(F)(F)F)nc(OCC5(CN6CCC(F)CC6)CC5)nc4c3F)c12 ; C#Cc1c(F)ccc2cc(O)cc(-c3ncc4c(N5CC6CCC(C5)N6C(=O)C(F)(F)F)nc(OC[C@H]5CCN6C[C@H](F)CC56)nc4c3F)c12 ; C#Cc1c(F)ccc2cc(O)cc(-c3ncc4c(N5CC6CCC(C5)N6C(=O)F)nc(OCC5(CN6CC7CC7C6)CC5)nc4c3F)c12]
or a pharmaceutically acceptable salt thereof.

34. A pharmaceutical composition comprising the compound of claim 1, or a pharmaceutically acceptable salt thereof; and a pharmaceutically acceptable carrier.

35. A method for treating cancer in a subject comprising administering to the subject and effective amount of the compound of claim 1, or a pharmaceutically acceptable salt thereof.
